(19) United States
(12) Patent Application Publication
(10) Pub. No.: US 2018/0340176 A1
(43) Pub. Date: Nov. 29, 2018

(54) CRISPR-CAS SGRNA LIBRARY

(71) Applicant: IFOM FONDAZIONE ISTITUTO FIRC DI ONCOLOGIA MOLECOLARE, Milano (MI) (IT)

(72) Inventor: Hiroshi ARAKAWA, Milano (MI) (IT)

(73) Assignee: IFOM FONDAZIONE ISTITUTO FIRC DI ONCOLOGIA MOLECOLARE, Milano (MI) (IT)

(21) Appl. No.: 15/774,686

(22) PCT Filed: Nov. 9, 2016
(86) PCT No.: PCT/EP2016/077165

§ 371 Date: May 9, 2018

(30) Foreign Application Priority Data
Nov. 9, 2015 (EP) 15193732.3

(51) Int. Cl. C12N 15/11; C12N 9/22; C12N 15/10

(52) U.S. Cl. CPC C12N 15/111; C12N 9/22; C12N 2330/31; C12N 15/1093; C12N 2310/20; C12N 15/1082

(57) ABSTRACT

The present invention refers to a method for obtaining a CRISPR-Cas system sgRNA library and to the use of the library to select individual cell knock outs that survive under a selective pressure and/or to identify the genetic basis of one or more biological or medical symptoms exhibited by a subject and/or to knocking out in parallel every gene in the genome.

### BACKGROUND OF THE INVENTION

The clustered regularly interspersed palindromic repeats (CRISPR) system is responsible for the acquired immunity of bacteria (1), which is shared among 40% of eubacteria and 90% of archaea (2). When bacteria are attacked by infectious agents, such as phages or plasmids, a subpopulation of the bacteria incorporates segments of the infectious DNA into a CRISPR locus as a memory of the bacterial adaptive immune system (1). If the bacteria are infected with the same pathogen, short RNA transcribed from the CRISPR locus is integrated into CRISPR-associated protein 9 (Cas 9), which acts as a sequence-specific endonuclease and eliminates the infectious pathogen (3).

CRISPR/Cas9 is available as a sequence-specific endonuclease (4, 5) that can cleave any locus of the genome if a guide RNA (gRNA) is provided. Indels on the genomic loci generated by non-homologous end joining (NHEJ) can knock out the corresponding gene (4, 5). By designing gRNA for the gene of interest, individual genes can be knocked out one-by-one (reverse genetics); however, this strategy is not helpful when the gene responsible for the phenomenon of interest is not identified. If a proper read out and selection method is available, phenotype screening (forward genetics) is an attractive alternative.

Recently, genome-scale pooled gRNA libraries have been applied for forward genetics screening in mammals (6-9). While phenotypic screening depends on the experimental set-up, the most straightforward method is screening based on the viability of mutant cell lines that are combined with either positive or negative selection. Negative selection screens for human gRNA libraries have identified essential gene sets involved in fundamental processes (6-8). Screens for resistance to nucleotide analogs or anti-cancer drugs successfully identified previously validated genes as well as novel targets (6-8). Thus, Cas9/gRNA screening has been shown to be a powerful tool for systematic genetic analysis in mammalian cells.

The gRNA for Streptococcus pyogenes (Sp) Cas9 can be designed as a 20-bp sequence that is adjacent to the protospacer adjacent motif (PAM) NGG (4, 5). Such a sequence can usually be identified from the coding sequence or locus of interest by bioinformatics techniques, but this approach is difficult for species with poorly annotated genetic information. Despite current advances in genome bioinformatics, annotation of the genetic information is incomplete in most species, except for well-established model organisms such as human, mouse, or yeast. While the diversity of species represents a diversity of special biological abilities, according to the organism, many of the genes encoding special abilities in a variety of species are left untouched, leaving an untapped gold mine of genetic information. Nevertheless, species-specific abilities are certainly beneficial due to possible transplantation in humans or applications for medical research.

If one wants to convert the mRNA into gRNA without prior knowledge of the target DNA sequences, the major challenges are to find the sequences flanking the PAM and to cut out the 20-bp fragment.

Shalem, O., Sanjana, N. E., Hartenian, E., Shi, X., Scott, D. A., Mikkelsen, T. S., Heckl, D., Ebert, B. L., Root, D. E., Doench, J. G. & Zhang, F. Genome-scale CRISPR-Cas9 knockout screening in human cells. Science 343, 84-87 (2014) show that lentiviral delivery of a genome-scale CRISPR-Cas9 knockout (GeCKO) library targeting 18,080 genes with 64,751 unique guide sequences enables both negative and positive selection screening in human cells. The disclosed sgRNA library was constructed using chemically synthesized oligonucleotides. Although the genome-scale sgRNA library is powerful, construction of an sgRNA in this way requires sufficient genetic information of the species in order to design guide sequences as well as enormous cost to synthesize a huge number of oligos. This makes difficult to create sgRNA library de novo in different biological model species. Wang, T., Wei, J. J., Sabatini, D. M. & Lander, E. S. Genetic screens in human cells using the CRISPR-Cas9 system. Science 343, 80-84 (2014) refers to a pooled, loss-of-function genetic screening approach suitable for both positive and negative selection that uses a genome-scale lentiviral single-guide RNA (sgRNA) library. sgRNA expression cassettes were stably integrated into the genome, which enabled a complex mutant pool to be tracked by massively parallel sequencing. A library containing 73,000 sgRNAs was used to generate knockout collections and performed screens in two human cell lines. A screen for resistance to the nucleotide analog 6-thioguanine identified all expected members of the DNA mismatch repair pathway, whereas another for the DNA topoisomerase II (TOP2A) poison etoposide identified TOP2A, as expected, and also cyclin-dependent kinase 6, CDK6. A negative selection screen for essential genes identified numerous gene sets corresponding to fundamental processes. Last, it was shown that sgRNA efficiency is associated with specific sequence motifs, enabling the prediction of more effective sgRNAs. Collectively, these results establish Cas9/sgRNA screens as a powerful tool for systematic genetic analysis in mammalian cells. The sgRNA library was constructed also using a huge number of chemically synthesized oligonucleotides.

Lane et al. developed an elegant approach using PAM-like restriction enzymes to generate guide libraries, which can label chromosomal loci in Xenopus egg extracts or can target the E. coli genome at high frequency (18).

The patent Application WO2015065964 relates to libraries, kits, methods, applications and screens used in functional genomics that focus on gene function in a cell and that may use vector systems and other aspects related to Clustered Regularly Interspaced Short Palindromic Repeats (CRISPR)-Cas systems and components thereof. The patent application also relates to rules for making potent single guide RNAs (sgRNAs) for use in CRISPR-Cas systems. Provided are genomic libraries and genome wide libraries, kits, methods of knocking out in parallel every gene in the genome, methods of selecting individual cell knock outs that survive under a selective pressure, methods of identifying the genetic basis of one or more medical symptoms exhibited by a patient, and methods for designing a genome-scale sgRNA library. The obtained sgRNA library is based on bioinformatics and cloning of a huge number of oligonucleotides.

The patent application US2014357523 refers to a method for fragmenting a genome. In certain embodiments, the method comprises: (a) combining a genomic sample containing genomic DNA with a plurality of Cas9-gRNA complexes, wherein the Cas9-gRNA complexes comprise a Cas9 protein and a set of at least 10 Cas9-associated guide RNAs that are complementary to different, pre-defined, sites in a genome, to produce a reaction mixture; and (b) incubating the reaction mixture to produce at least 5 fragments of the genomic DNA. Also provided is a composition comprising at least 100 Cas9-associated guide RNAs that are each complementary to a different, pre-defined, site in a genome. Kits for performing the method are also provided. In addition, other methods, compositions and kits for manipulating nucleic acids are also provided. This approach aims fragmentation of the target of initially identified genes (reverse genetics), and is not related to a construction of a genome-scale sgRNA library.

The clustered regularly interspersed palindromic repeats (CRISPR)/Cas9 system is a powerful tool for genome editing^{4, 5} that can be used to construct a guide RNA (gRNA) library for genetic screening^{6, 7}. For gRNA design, one must know the sequence of the 20-mer flanking the protospacer adjacent motif (PAM)^{4, 5}, which seriously impedes making gRNA experimentally.

Therefore, it is still felt the need of a method for obtaining a sgRNA library by molecular biological techniques without relying on bioinformatics and without requiring prior knowledge about the target DNA sequences, making the method applicable to any species.

### SUMMARY OF THE INVENTION

Inventor herein describes a method to construct a gRNA library by molecular biological techniques, without relying on bioinformatics, and which allows forward genetics screening of any species, independent of their genetic characterization. Since the present method is not based on bioinformatics, it is possible to create guide sequences even from unknown genetic information.

Briefly, one synthesizes cDNA from the mRNA sequence using a semi-random primer containing a complementary sequence to the PAM and then cuts out the 20-mer adjacent to the PAM using type IIS and type III restriction enzymes to create a gRNA library.

The described approach does not require prior knowledge about the target DNA sequences, making it applicable to any species, whereas gRNA libraries generated this way are at least 100-fold cheaper than oligo cloning-based libraries.

It is therefore an object of the invention the use of a semi-random primer comprising a protospacer adjacent motif (PAM)-complementary sequence to produce a clustered regularly interspersed short palindromic repeats (CRISPR)-Cas single-guide RNA (sgRNA) library or a sgRNA or a guide sequence.

Preferably, said semi-random primer is used as cDNA synthesis primer to produce a clustered regularly interspersed short palindromic repeats (CRISPR)-Cas single-guide RNA (sgRNA) library or a sgRNA or a guide sequence.

Said semi-random primer is preferably 4 to 10 nucleotides long.

The PAM-complementary sequence is preferably complementary to a PAM sequence specific for S. progenies (Sp) Cas9, Neisseria meningitidis (NM) Cas9, Streptococcus thermophilus (ST) Cas9 or Treponema denticola (TD) Cas9, orthologues, homologues or variants thereof.

Said PAM-complementary sequence is a sequence which is preferably substantially complementary or more preferably perfectly complementary to a PAM sequence.

In a preferred embodiment of the invention the PAM sequence is selected from the group consisting of: 5′-NGG-3′, 5′-NNNNGATT-3′, 5′-NNAGAAW-3′ and 5′-NAAAAC-3′, orthologues, homologues or variants thereof, wherein N is a nucleotide selected from C, G, A and T.

Said PAM-complementary sequence preferably comprises the sequence 5-CCN-3′, wherein N is a nucleotide selected from C, G, A and T, said primer being preferably phosphorylated at the 5′ terminus.

Preferably, the semi-random primer comprises or has essentially the sequence of SEQ ID NO: 1 (5′-NNNCCN-3′).

A further object of the invention is a method for obtaining a guide sequence comprising the following steps:

a) DNA synthesis from a RNA or a DNA using a semi-random primer as defined in any one of previous claims,

b) generation of guide sequences by molecular biological methods.

The guide sequence is preferably generated from mass RNA or DNA by molecular biological methods including cDNA synthesis and/or restriction digest and/or DNA ligation and/or PCR.

Said guide sequence is preferably generated cutting the synthetized DNA to obtain a guide sequence. The obtained guide sequence preferably consists of 20 base pairs.

The cutting is preferably carried out with at least one type III restriction enzyme and/or a type IIS restriction enzyme.

Preferably the cutting is carried out with enzymes that cleave 25/27 and/or 14/16 base pairs away from their recognition site.

The method of the invention preferably further comprises, before cutting the synthetized DNA, a step wherein the synthetized DNA is modified by addition of restriction sites for said restriction enzymes.

In the a preferred embodiment of the method of the invention, step b) comprises the following steps:

i) modification of synthetized DNA by addition:
to the 5′ end of the synthetized DNA of a linker sequence comprising a type III first restriction site and/or a type IIS second restriction site

and/or
to the 3′ end of the synthetized DNA of a linker sequence comprising a type IIS third restriction site and/or a type III fourth restriction sites

ii) cutting of the modified DNA as above defined.

In a preferred embodiment of the invention, the synthetized DNA is modified by the addition:
to the 5′ end of the synthetized DNA of a linker sequence comprising a type III first restriction site and/or a type IIS second restriction site

and
to the 3′ end of the synthetized DNA of a linker sequence comprising a type IIS third restriction site and/or a type III fourth restriction sites.

More preferably, the synthetized DNA is modified by the addition:
to the 5′ end of the synthetized DNA of a linker sequence comprising a type III first restriction site and
to the 3′ end of the synthetized DNA of a linker sequence comprising a type IIS third restriction site and a type III fourth restriction sites.

Preferably, the synthetized DNA is a dsDNA.

Preferably, the RNA is a mRNA, more preferably a purified poly(A)RNA.

The type III restriction site is preferably selected from the group consisting of: EcoP15I or EcoP1I restriction site, more preferably the type III restriction site is EcoP15I.

The type IIS restriction sites is preferably selected from the group consisting of: AcuI, BbvI, BpmI, FokI, GsuI, BsgI, Eco57I, Eco57MI, BpuEI or MmeI restriction site, more preferably the type IIS restriction site is AcuI.

In a preferred embodiment of the invention, the linker sequence at the 5′ end of the synthetized DNA preferably comprises an EcoP15I restriction site.

Preferably, the linker sequence at the 3′ end of the synthetized DNA comprises an EcoP15I restriction site and an AcuI restriction site.

In a preferred embodiment, the linker sequence at the 5′ end of the synthetized DNA further comprises a fifth restriction site, preferably BglII restriction site, and/or the linker sequence at the 3′ end of the synthetized DNA further comprises a sixth restriction site, preferably a XbaI restriction site.

Other suitable restriction sites may be used instead of BglII or XbaI.

In a preferred embodiment the linker at the 3′ end of the synthetized DNA is:

```
            EcoP15I AcuI XbaI
5′ CTGCTGACTTCAGTGGTTCTAGAGGTGTCCAAC 3′
(SEQ ID NO: 284)
3′ p TGACGACTGAAGTCACCAAGATCTCCACAGGTTG 5′
(SEQ ID NO: 3)
or
        Eco P15I Acu I Xba I
5′-p CTGCTGACTTCAGTGGTTCTAGAGGTGTCCAA-3′
(SEQ ID NO: 2)
3′-TGACGACTGAAGTCACCAAGATCTCCACAGGTTG-5′
(SEQ ID NO: 3)
```

Preferably, the above method further comprises a step i′) wherein the modified DNA is digested with the specific type III restriction enzyme.

More preferably, the method further comprising a step i″) wherein the to the 5′ end of the digested DNA is added a further linker sequence comprising a seventh restriction site which is a cloning site for the gRNA expression vector and a eight restriction site, preferably a AatII restriction site, and the DNA is then optionally digested with the specific restriction enzyme for the fifth restriction site at the 5′, preferably BglII restriction enzyme.

Other suitable restriction sites may be used instead of AatII or BglII.

Preferably the restriction site which is a cloning site is a BsmBI site.

The above defined method preferably further comprises a step i′″) wherein the DNA is amplified, preferably by PCR, and digested with the specific type IIS restriction enzyme for the third restriction site at the 3′ and optionally with the specific restriction enzyme for the sixth restriction site, preferably with XbaI.

The above defined method preferably further comprises a step i″″) wherein the guide sequence fragment is purified from the digested DNA and ligated with a further linker sequence at the 3′ end comprising a restriction site which is a cloning site for the gRNA expression vector and optionally a ninth restriction site, preferably AatII restriction site.

The above defined method preferably further comprises a step i′″″) wherein the DNA is amplified, preferably by PCR, and digested with the specific restriction enzyme for the cloning site and optionally with the specific restriction enzyme for the ninth restriction site, preferably with AatII.

In a preferred embodiment, 25-bp fragments are then purified.

Another object of the invention is an isolated guide sequence obtainable by the method of the invention.

A further object of the invention is an isolated sgRNA comprising the RNA corresponding to the isolated guide sequence as above defined.

Another object of the invention is a method for obtaining a CRISPR-Cas system sgRNA library comprising cloning the guide sequences as above defined into a sgRNA expression vector and transforming said vector into a competent cell to obtain a CRISP-Cas system sgRNA library.

Preferably, the expression vector is a lentivirus, and/or the vector comprises a species specific functional promoter, preferably a pol III promoter, more preferably U6 promoter and/or a gRNA scaffold sequence.

A further object of the invention is a CRISPR-Cas system sgRNA library obtainable by above defined method.

Another object of the invention is a library comprising a plurality of CRISPR-Cas system guide sequences that target a plurality of target sequences in genomic loci of a plurality of genes, wherein said targeting results in a knockout of gene function, wherein the unique CRISPR-Cas system guide sequences are obtained by using a semi-random primer as above defined in.

Said plurality of genes are preferably Gallus gallus genes.

Another object of the invention is an isolated sgRNA or an isolated guide sequence selected from the library of the invention.

A further object of the invention is the use of the guide sequence as above defined or of the CRISPR-Cas system sgRNA library as above defined or of the sgRNA as above defined, for functional genomic studies, preferably to select individual cell knock outs that survive under a selective pressure and/or to identify the genetic basis of one or more biological or medical symptoms exhibited by a subject and/or to knocking out in parallel every gene in the genome.

Other objects of the invention are a kit comprising the semi-random primer as above defined for carrying out the above defined method, a kit comprising the guide sequence as above defined or the CRISPR-Cas system sgRNA library as above defined or the sgRNA as above defined; a kit comprising one or more vectors, each vector comprising at least one guide sequence according to the invention, wherein the vector comprises a first regulatory element operably linked to a tracr mate sequence and a guide sequence upstream of the tracr mate sequence, wherein when expressed, the guide sequence directs sequence-specific binding of a CRISPR complex to a target sequence in a eukaryotic cell, wherein the CRISPR complex comprises a Cas9 enzyme complexed with (1) the guide sequence and (2) the tracr mate sequence that is hybridized to a tracr sequence; an isolated DNA molecule encoding the guide sequence as above defined or the sgRNA as above defined; a vector comprising a DNA molecule as above defined; an isolated host cell comprising the DNA molecule as above defined or the vector as above defined, the isolated host cell as above defined which has been transduced with the library as above defined.

The primer used in the present invention is a semi-random primer, which is composed of mixture of fixed and random sequence.

In one aspect, the invention provides a library comprising a plurality of CRISPR-Cas sytem guide sequence that are capable of targeting a plurality of target sequences in genomic loci, wherein said targeting results in a knockout of gene function.

The invention also comprehends kit comprising the library of the invention. In certain aspects, wherein the kit comprises a single container comprising vectors comprising the library of the invention. In other aspects, the kit comprises a single container comprising plasmids comprising the library of the invention. The invention also comprehends kits comprising a panel comprising a selection of unique CRISPR-Cas system guide sequences from the library of the invention, wherein the selection is indicative of a particular physiological condition. The kit may also comprise a panel comprising a selection of unique CRISPR-Cas system guide RNAs comprising guide sequences from the library of the invention, wherein the selection is indicative of a particular physiological condition. In preferred embodiments, the targeting is of about 100 or more sequences, about 1000 or more sequences or about 20,000 or more sequences or the entire genome; in other embodiments a panel of target sequences is focused on a relevant or desirable pathway, such as an immune pathway or cell division. In one aspect, the invention provides a genome wide library comprising a plurality of unique CRISPR-Cas system guide sequences that are capable of targeting a plurality of target sequences in genomic loci of a plurality of genes, wherein said targeting results in a knockout of gene function.

In certain embodiments of the invention, the guide sequences are capable of targeting a plurality of target sequences in genomic loci of a plurality of genes selected from the entire genome, in embodiments, the genes may represent a subset of the entire genome; for example, genes relating to a particular pathway (for example, an enzymatic pathway) or a particular disease or group of diseases or disorders may be selected. One or more of the genes may include a plurality of target sequences; that is, one gene may be targeted by a plurality of guide sequences. In certain embodiments, a knockout of gene function is not essential, and for certain applications, the invention may be practiced where said targeting results only in a knockdown of gene function.

However, this is not preferred.

In another aspect, the invention provides for a method of knocking out in parallel every gene in the genome, the method comprising contacting a population of cells with a composition comprising a vector system comprising one or more packaged vectors comprising

a) a first regulatory element operably linked to a CRISPR-Cas system chimeric RNA (chiRNA) polynucleotide sequence that targets a DNA molecule encoding a gene product, wherein the polynucleotide sequence comprises

(a) a guide sequence capable of hybridizing to a target sequence,

(b) a tracr mate sequence, and

(c) a tracr sequence, and

b) a second regulatory element operably linked to a Cas protein and a selection marker, wherein components (a) and (b) are located on same or different vectors of the system, wherein each cell is transduced or transfected with a single packaged vector,

selecting for successfully transduced cells,

wherein when transcribed, the tracr mate sequence hybridizes to the tracr sequence and the guide sequence directs sequence-specific binding of a CRISPR complex to a target sequence in the genomic loci of the DNA molecule encoding the gene product,

wherein the CRISPR complex comprises a CRISPR enzyme complexed with (1) the guide sequence that is hybridized to the target sequence, and (2) the tracr mate sequence that is hybridized to the tracr sequence,

wherein the guide sequence is selected from the library of the invention,

wherein the guide sequence targets the genomic loci of the DNA molecule encoding the gene product and the CRISPR enzyme cleaves the genomic loci of the DNA molecule encoding the gene product and whereby each cell in the population of cells has a unique gene knocked out in parallel.

The present methods and uses may be carried out in any kind of cells or organisms. In preferred embodiments, the cell is a eukaryotic cell. The eukaryotic cell may be a plant or animal cell; for example, algae or microalgae; invertebrates, such as planaria; vertebrate, preferably mammalian, including murine, ungulate, primate, human; insect. In further embodiments the vector is a lenti virus, an adenovirus or an AAV and/or the first regulatory element is a U6 promoter and/or the second regulatory element is an EPS promoter or a doxycycline inducible promoter, and/or the vector system comprises one vector and/or the CRISPR enzyme is Cas9. In aspects of the invention the cell is a eukaryotic cell, preferably a human cell. In a further embodiment, the cell is transduced with a multiplicity of infection (MOT) of 0.3-0.75, preferably, the MOI has a value close to 0.4, more preferably the MOI is 0.3 or 0.4.

The invention also encompasses methods of selecting individual cell knock outs that survive under a selective pressure, the method comprising

contacting a population of cells with a composition comprising a vector system comprising one or more packaged vectors comprising

a) a first regulatory element operably linked to a CRISPR-Cas system chimeric RNA (chiRNA) polynucleotide sequence that targets a DNA molecule encoding a gene product, wherein the polynucleotide sequence comprises

(a) a guide sequence capable of hybridizing to a target sequence,

(b) a tracr mate sequence, and

(c) a tracr sequence, and

b) a second regulatory element operably linked to a Cas protein and a selection marker, wherein components (a) and (b) are located on same or different vectors of the system, wherein each cell is transduced or transfected with a single packaged vector,

selecting for successfully transduced cells,

wherein when transcribed, the tracr mate sequence hybridizes to the tracr sequence and the guide sequence directs sequence-specific binding of a CRISPR complex to a target sequence in the genomic loci of the DNA molecule encoding the gene product,

wherein the CRISPR complex comprises a CRISPR enzyme complexed with (1) the guide sequence that is hybridized to the target sequence, and (2) the tracr mate sequence that is hybridized to the tracr sequence,

wherein the guide sequence is selected from the library of the invention,

wherein the guide sequence targets the genomic loci of the DNA molecule encoding the gene product and the CRISPR enzyme cleaves the genomic loci of the DNA molecule encoding the gene product, whereby each cell in the population of cells has a unique gene knocked out in parallel, applying the selective pressure,

and selecting the cells that survive under the selective pressure.

In preferred embodiments, the selective pressure is application of a drug, FACS sorting of cell markers or aging and/or the vector is a lentivirus, a adenovirus or a AAV and/or the first regulatory element is a U6 promoter and/or the second regulatory element is an EFS promoter or a doxycycline inducible promoter, and/or the vector system comprises one vector and/or the CRISPR enzyme is Cas9. In a further embodiment the cell is transduced with a multiplicity of infection (MOI) of 0.3-0.75, preferably, the MOI has a value close to 0.4, more preferably the MOI is 0.3 or 0,4. In aspects of the invention the cell is a eukaryotic cell. The eukaryotic cell may be a plant or animal cell; for example, algae or microalgae; invertebrate; vertebrate, preferably mammalian, including murine, ungulate, primate, human; insect. Preferably the cell is a human cell. In preferred embodiments of the invention, the method further comprises extracting DNA and determining the depletion or enrichment of the guide sequences by deep sequencing.

In other aspects, the invention encompasses methods of identifying the genetic basis of one or more medical symptoms exhibited by a subject, the method comprising

obtaining a biological sample from the subject and isolating a population of cells having a first phenotype from the biological sample;

contacting the cells having the first phenotype with a composition comprising a vector system comprising one or more packaged vectors comprising

a) a first regulatory element operably linked to a CRISPR-Cas system chimeric RNA (chiRNA) polynucleotide sequence that targets a DN A molecule encoding a gene product, wherein the polynucleotide sequence comprises

(a) a guide sequence capable of hybridizing to a target sequence,

(b) a tracr mate sequence, and

(c) a tracr sequence, and

b) a second regulatory element operably linked to a Cas protein and a selection marker, wherein components (a) and (b) are located on same or different vectors of the system, wherein each cell is transduced or transfected with a single packaged vector,

selecting for successfully transduced cells,

wherein when transcribed, the tracr mate sequence hybridizes to the tracr sequence and the guide sequence directs sequence-specific binding of a CRISPR complex to a target sequence in the genomic loci of the DNA molecule encoding the gene product,

wherein the CRISPR complex comprises a CRISPR enzyme complexed with (1) the guide sequence that is hybridized to the target sequence, and (2) the tracr mate sequence that is hybridized to the tracr sequence,

wherein the guide sequence is selected from the library of the invention,

wherein the guide sequence targets the genomic loci of the DN A molecule encoding the gene product and the CRISPR enzyme cleaves the genomic loci of the DNA molecule encoding the gene product, whereby each cell in the population of cells has a unique gene knocked out in parallel,

applying a selective pressure, selecting the cells that survive under the selective pressure,

determining the genomic loci of the DNA molecule that interacts with the first phenotype and identifying the genetic basis of the one or more medical symptoms exhibited by the subject.

In preferred embodiments, the selective pressure is application of a drug, FACS sorting of cell markers or aging and/or the vector is a lenti virus, an adenovirus or an AAV and/or the first regulatory element is a U6 promoter and/or the second regulatory element is an EFS promoter or a doxycycline inducible promoter, and/or the vector system comprises one vector and/or the CRISPR enzyme is Cas9. In a further embodiment the cell is transduced with a multiplicity of infection (MOI) of 0.3-0.75, preferably, the MO I has a value close to 0.4, more preferably the MOI is 0.3 or 0.4. in aspects of the invention the cell is a eukaryotic cell, preferably a human cell.

In an aspect, the invention provides a non-human eukaryotic organism; preferably a multicellular eukaryotic organism, comprising a eukaryotic host cell according to any of the described embodiments in which a candidate gene is knocked down or knocked out. Preferably the gene is knocked out. In other aspects, the invention provides a eukaryotic organism; preferably a multicellular eukaryotic organism, comprising a eukaryotic host cell which has been altered according to any of the described embodiments. The organism in some embodiments of these aspects may be an animal; for example a mammal. Also, the organism may be an arthropod such as an insect. The organism also may be a plant. Further, the organism may be a fungus. In some embodiments, the invention provides a set of non-human eukaryotic organisms, each of which comprises a eukaryotic host cell according to any of the described embodiments in which a candidate gene is knocked down or knocked out. In preferred embodiments, the set comprises a plurality of organisms, in each of which a different gene is knocked down or knocked out.

In some embodiments, the CRISPR enzyme comprises one or more nuclear localization sequences of sufficient strength to drive accumulation of said CRISPR enzyme in a detectable amount in the nucleus of a eukaryotic cell. In some embodiments, the CRISPR enzyme is a type II CRISPR system enzyme. In some embodiments, the CRISPR enzyme is a Cas9 enzyme. In some embodiments, the Cas9 enzyme is S. pneumoniae, S. pyogenes or S. thermophilus Cas9, and may include mutated Cas9 derived from these organisms. The enzyme may be a Cas9 homolog or ortholog. In some embodiments, the CRISPR enzyme is codon—optimized for expression in a eukaryotic cell. In some embodiments, the CRISPR enzyme directs cleavage of one or two strands at the location of the target sequence, in some embodiments, the CRISPR enzyme lacks DNA strand cleavage activity. In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length. In an advantageous embodiment the guide sequence is 20 nucleotides in length.

In a preferred embodiment, the invention has advantageous pharmaceutical application, e.g., the invention may be harnessed to test how robust any new drug designed to kill cells (eg. chemotherapeutic) is to mutations that KO genes. Cancers mutate at an exceedingly fast pace and the libraries and methods of the invention may be used in functional genomic screens to predict the ability of a chemotherapy to be robust to “escape mutations”.

According to one aspect of the invention, a method of altering a eukaryotic cell is providing including transfecting the eukaryotic cell with a nucleic acid encoding RNA complementary to genomic DNA of the eukaryotic cell, transfecting the eukaryotic cell with a nucleic acid encoding an enzyme that interacts with the RNA and cleaves the genomic DNA in a site specific manner, wherein the cell expresses the RNA and the enzyme, the RNA binds to complementary genomic DNA and the enzyme cleaves the genomic DNA in a site specific manner. Said nucleic acid encoding RNA complementary to genomic DNA is preferably the guide sequence of the present invention. Preferably, the enzyme is Cas9 or modified Cas9 or a homolog of Cas9. More preferably, the eukaryotic cell is a yeast cell, a plant cell or a mammalian cell. According to one aspect, the RNA includes between about 20 to about 100 nucleotides.

According to one aspect of the invention, to direct Cas9 to cleave sequences of interest, crRNA-tracrRNA fusion transcripts are expressed, herein also referred to as “guide RNAs” (gRNAs), from the human U6 polymerase III promoter. gRNAs may be directly transcribed by the cell.

The invention also provides a method of generating a gene knockout cell library comprising introducing into each cell in a population of cells a vector system of one or more vectors that may comprise an engineered, non-naturally occurring CRISPR-Cas system comprising I. a Cas protein, and II. one or more guide RNAs of the library of the invention, wherein components I and II may be on the same or on different vectors of the system, integrating components I and II into each cell, wherein the guide sequence targets a unique gene in each cell, wherein the Cas protein is operably linked to a regulatory element, wherein when transcribed, the guide RNA comprising the guide sequence directs sequence-specific binding of a CRISPR-Cas system to a target sequence in the genomic loci of the unique gene, inducing cleavage of the genomic loci by the Cas protein, and confirming different knockout mutations in a plurality of unique genes in each cell of the population of cells thereby generating a gene knockout cell library. In an embodiment of the invention, the Cas protein is a Cas9 protein. In another embodiment, the one or more vectors are plasmid vectors. In a further embodiment, the regulatory element operably linked to the Cas protein is an inducible promoter, e.g. a doxycycline inducible promoter. The invention comprehends that the population of cells is a population of eukaryotic cells, and in a preferred embodiment, the population of cells is a population of embryonic stem (ES) cells, preferably non human. In another aspect the invention provides for use of genome wide libraries for functional genomic studies. Such studies focus on the dynamic aspects such as gene transcription, translation, and protein-protein interactions, as opposed to the static aspects of the genomic information such as DNA sequence or structures, though these static aspects are very important and supplement one's understanding of cellular and molecular mechanisms. Functional genomics attempts to answer questions about the function of DNA at the levels of genes, RNA transcripts, and protein products. A key characteristic of functional genomics studies is a genome-wide approach to these questions, generally involving high-throughput methods rather than a more traditional “gene-by-gene” approach. Given the vast inventory of genes and genetic information it is advantageous to use genetic screens to provide information of what these genes do, what cellular pathways they are involved in and how any alteration in gene expression can result in particular biological process.

Preferably, delivery is in the form of a vector which may be a viral vector, such as a lenti- or baculo- or preferably adeno-viral/adeno-associated viral vectors, but other means of delivery are known (such as yeast systems, microvesicles, gene guns/means of attaching vectors to gold nanoparticles) and are provided. A vector may mean not only a viral or yeast system (for instance, where the nucleic acids of interest may be operably linked to and under the control of (in terms of expression, such as to ultimately provide a processed RNA) a promoter), but also direct delivery of nucleic acids into a host cell. While in herein methods the vector may be a viral vector and this is advantageously an AAV, other viral vectors as herein discussed can be employed, such as lentivirus. For example, baculoviruses may be used for expression in insect cells. These insect cells may, in turn be useful for producing large quantities of further vectors, such as AAV or lentivirus vectors adapted for delivery of the present invention. Also envisaged is a method of delivering the present CRISP enzyme comprising delivering to a cell mRNA encoding the CRISPR enzyme. It will be appreciated that in certain embodiments the CRISPR enzyme is truncated, and/or comprised of less than one thousand amino acids or less than four thousand amino acids, and/or is a nuclease or nickase, and/or is codon-optimized, and/or comprises one or more mutations, and/or comprises a chimeric CRISPR enzyme, and/or the other options as herein discussed. AAV and lentiviral vectors are preferred.

Viral delivery: The CRISPR enzyme, for instance a Cas9, and/or any of the present RNAs, for instance a guide RNA, can be delivered using adeno associated virus (AAV), lentivirus, adenovirus or other viral vector types, or combinations thereof. Cas9 and one or more guide RNAs can be packaged into one or more viral vectors. In some embodiments, the viral vector is delivered to the tissue of interest by, for example, an intramuscular injection, while other times the viral delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector chose, the target cell, organism, or tissue, the general condition of the subject to be treated, the degree of transformation/modification sought, the administration route, the administration mode, the type of transformation/modification sought, etc.

One aspect of the invention comprehends a genome wide library that may comprise a plurality of CRISPR-Cas system guide RNAs that may comprise guide sequences that are capable of targeting a plurality of target sequences in a plurality of genomic loci, wherein said targeting results in a knockout of gene function. This library may potentially comprise guide RNAs that target each gene in the genome of an organism. In some embodiments of the invention the organism or subject is a eukaryote (including mammal including human) or a non-human eukaryote or a non-human animal or a non-human mammal. In some embodiments, the organism or subject is a non-human animal, and may be an arthropod, for example, an insect, or may be a nematode. In some methods of the invention the organism or subject is a plant. In some methods of the invention the organism or subject is a mammal or a non-human mammal. A non-human mammal may be for example a rodent (preferably a mouse or a rat), an ungulate, or a primate. In some methods of the invention the organism or subject is algae, including microalgae, or is a fungus.

The length and sequence of the semi-random primer may be modified according to guide sequence generation strategy. EcoP15I is currently the most suitable type III restriction enzyme for the method of the invention. This enzyme cleaves 27 bp separated position from its recognition sequence, and a guide sequence will need the minimum length of 17 bp. Since a semi-random primer bridges the restriction site and the guide sequence, maximum length of a semi-random primer can be 10 mer. The minimum length of a cDNA synthesis primer can be 4 mer. Thus a semi-random primer containing PAM can have variation between 4 and 10 mer of N (0-7) CC N (1-8). While this sequence is optimized for Sp Cas9, the sequence of a semi-random primer can be further customized depending on PAM sequence of Cas9 from different species.

In order to recognize the target sequence, Cas9 requires a protospacer adjacent motif (PAM) neighboring the target sequence. The PAM sequence is required in the target DNA but not in the gRNA sequence. The PAM sequences vary depending on Cas9 derived from different bacterial species. For example, NGG is the PAM sequence for S. progenies (Sp) Cas9, which is the endonuclease for the most widely used type II CRISPR system. PAM sequences of Cas9 from other species are, for example, NNNNGATT for Neisseria meningitidis (NM), NNAGAAW for Streptococcus thermophilus (ST) and NAAAAC for Treponema denticola (TD).

The sequence of the semi-random primer can be changed depending on experimental design. In an alternative preferred embodiment the sequence of the semi-random primer is 5′ NNCCNN 3′. PAMs are different among deferent species-derived Cas9, and the semi-random primer may be modified accordingly.

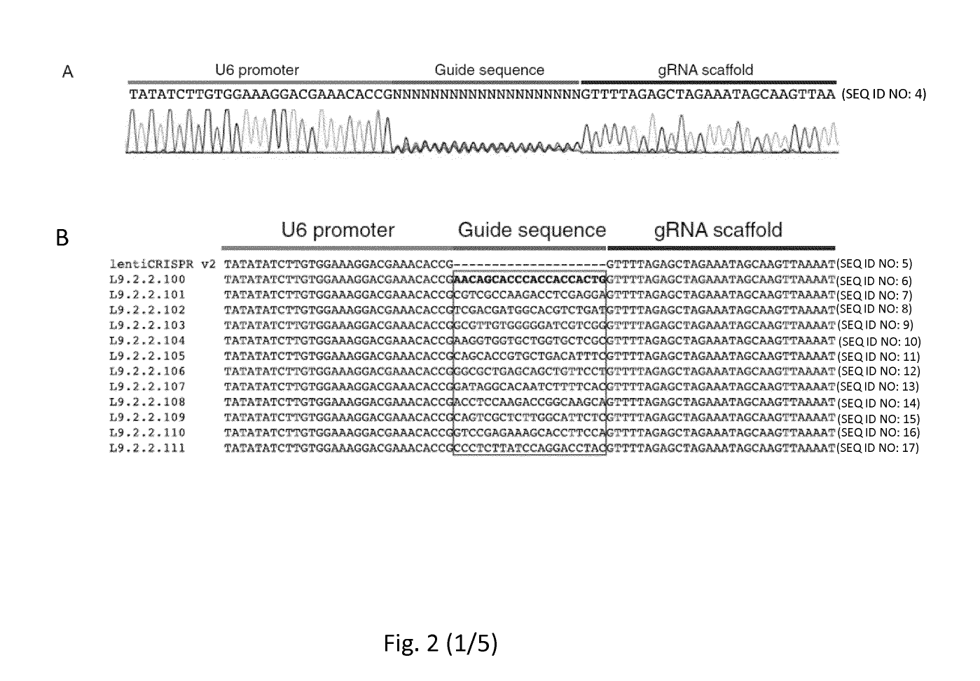

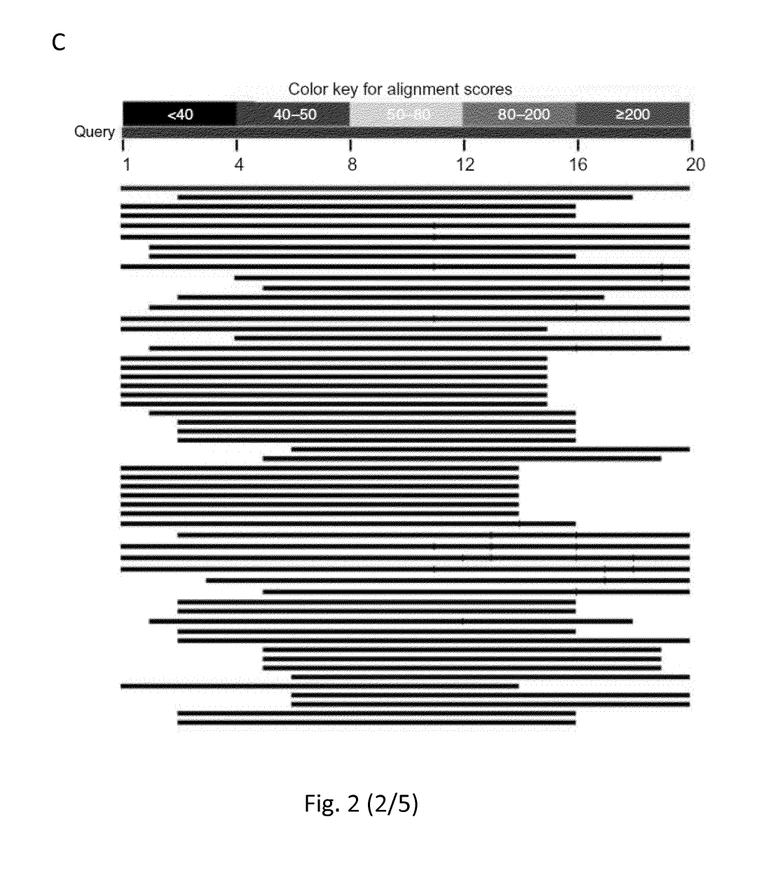

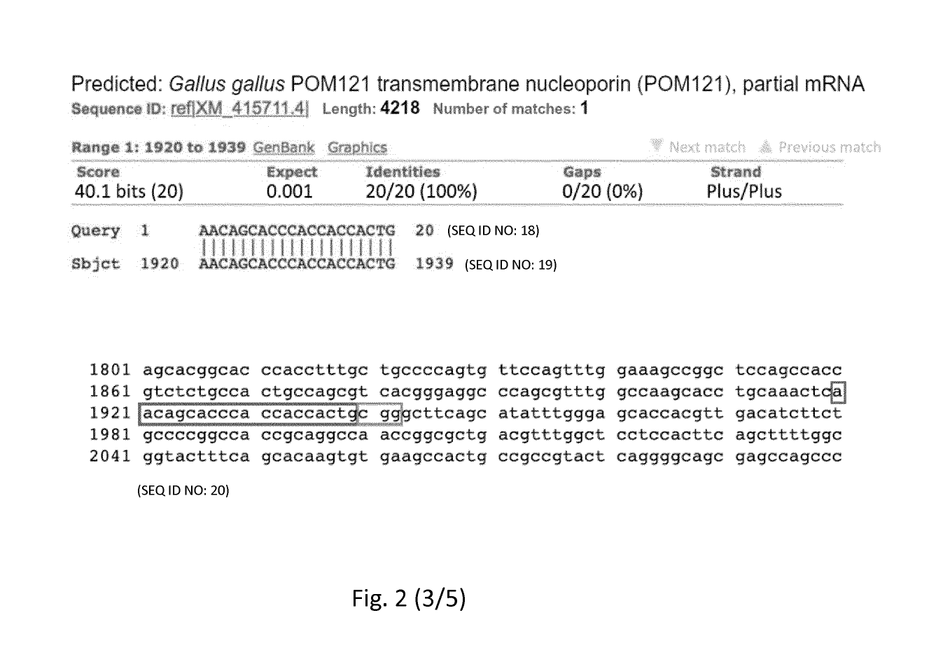

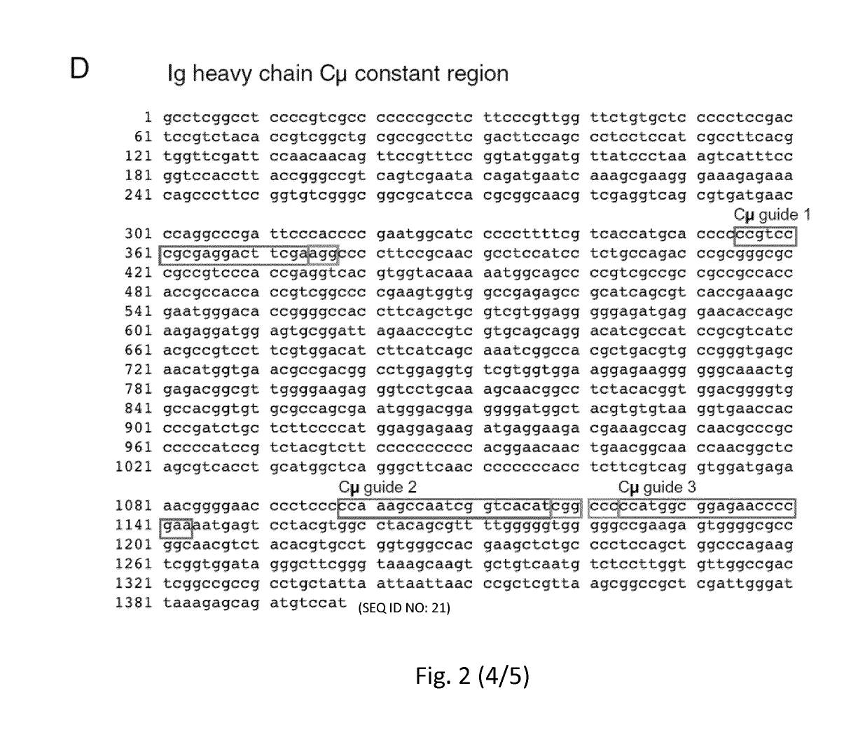

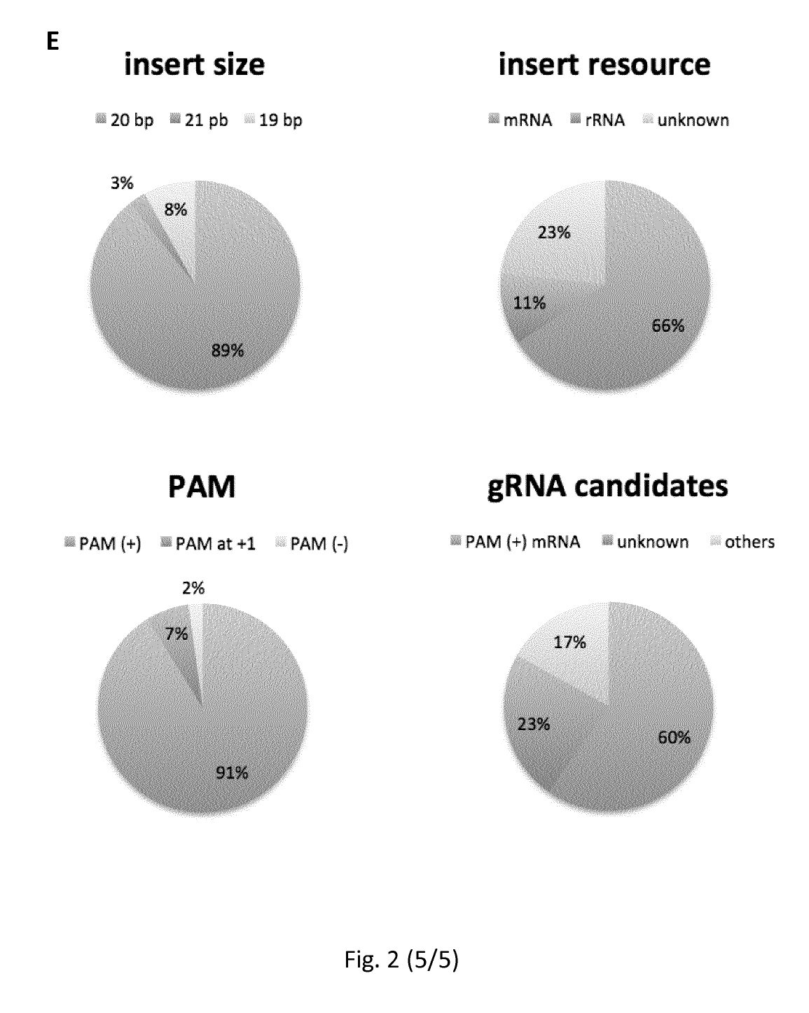

To use the CRISPR system, gRNA needs to be expressed and to be recruited into Cas9. In a gRNA expression vector, gRNA expression may be driven by a promoter which functions in a specific species or cell type. Since pol III promoter is suitable for expression of defined length of short RNA, typically pol III promoter like U6 promoter is used for gRNA expression. In a gRNA expression vector, the guide sequence cloning site will be followed by the gRNA scaffold sequence (e.g. the sequence as mentioned in FIG. 2b or its proper variants). The gRNA scaffold is folded and integrated into Cas9, thus allowing recruitment and proper positioning of the gRNA into Cas9 endonuclease. In this case, another vector coding for Cas9 will be used.

With respect to general information on CRISPR-Cas Systems, components thereof and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, 8,795,965, 8,865,406 and 8,871,445; US Patent Publications US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486); PCT Patent Publications WO 2014/093661 (PCT/US2013/074743), WO 2014/093694 (PCT/US2013/074790), WO 2014/093595 (PCT/US2013/074611), WO 2014/09371 8 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO2014/093701 (PCT/US2013/074800), and WO2014/018423 (PCT/US2013/051418); U.S. provisional patent applications 61/961,980 and 61/963,643 each entitled FUNCTIONAL GENOMICS USING CRISPR-CAS SYSTEMS, COMPOSITIONS, METHODS, SCREENS AND APPLICATIONS THEREOF, filed Oct. 28 and Dec. 9, 2013 respectively; PCT/US2014/041806, filed Jun. 10, 2014, U.S. provisional patent applications 61/836,123, 61/960,777 and 61/995,636, filed on Jun. 17, 2013, Sep. 25, 2013 and Apr. 15, 2014, and PCT/US 13/74800, filed Dec. 12, 2013: Reference is also made to US provisional patent applications 61/736,527, 61/748,427, 61/791,409 and 61/835,931, filed on Dec. 12, 2012, Jan. 2, 2013, Mar. 15, 2013 and Jun. 17, 2013, respectively. Reference is also made to U.S. provisional applications 61/757,972 and 61/768,959, filed on Jan. 29, 2013 and Feb. 25, 2013, respectively. Reference is also made to U.S. provisional patent applications 61/835,931, 61/835,936, 61/836,127, 61/836,101, 61/836,080 and 61/835,973, each filed Jun. 17, 2013. Each of these applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these applications and the appln cited documents) are incorporated herein by reference to the same extent as if each individual document was specifically and individually indicated to be incorporated by reference. Citations for documents cited herein may also be found in the foregoing herein-cited documents, as well as those herein below cited.

Also with respect to general information on CRISPR-Cas Systems, mention is made of:
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M, Cheng A W., Zhang F., Jaenisch R. Cell May 9; 153(4):910-8 (2013);
Optical control of mammalian endogenous transcription and epi genetic states. onermann S, Brigham M D, Trevino A E, Hsu P D, Heidenreich M, Cong L, Piatt R J, Scott D A, Church G M, Zhang F. Nature. 2013 Aug. 22; 500(7463):472-6. doi: 10.1038/Naturel 2466. Epub 2013 Aug. 23;
Double Niching by RNA-Guided CRISPR Cas for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: 80092-8674(13)01015-5. (2013/;
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol 2013 September; 31(9):827-32. doi: 10.1038/nbt2647. Epub 2013 Jul. 21;
Genome engineering using the CRISPR-Cas9 system. Ran, F A., Hsu, P D., Wright, J., Agarwala, V., Scott, D A., Zhang, F. Nature Protocols November; 8(1 1):2281-308. (2013);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12, (2013). [Epub ahead of print]; Crystal structure of cas9 in complex with guide RNA and target DNA. Nishimasu, F L, Ran, F A., Hsu, P D., Konermann, S., Shehata, S I, Dohmae, Ishitatii, R., Zhang, F., Nureki, O. Cell February 27. (2014). 156(5):935-49;
Genome-wide binding of the CRISPR endonuclease Cas9 in mammalian cells. Wu X., Scott D A., Kriz A J., Chiu A C, Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889,
Development and Applications of CRISPR-Cas 9 for Genome Engineering, Hsu et al, Cell 157, 1262-1278 (Jun. 5, 2014) (Hsu 2014),
Genetic screens in human cells using the CRISPR/Cas9 system, Wang et al., Science. 2014 January 3; 343(6166): 80-84. doi: 10.1126/science.1246981, and
Rational design of highly active sgRNAs for CRISPR-Cas9-mediated gene inactivation, Doench et al., Nature Biotechnology published online 3 Sep. 2014; doi: 10.1038/nbt.3026. each of which is incorporated herein by reference.

### DETAILED DESCRIPTION OF THE INVENTION

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. A polynucleotide may comprise one or more modified nucleotides, such as methylated nucleotides and nucleotide analogs.

In aspects of the invention the terms “chimeric RNA”, “chimeric guide RNA”, “guide RNA”, “single guide RNA” and “synthetic guide RNA” are used interchangeably and refer to the polynucleotide sequence comprising the guide sequence, the tracr sequence and the tracr mate sequence.

The term “guide sequence” refers to the about 20 bp sequence within the guide RNA that specifies the target site and may be used interchangeably with the terms “guide” or “spacer”. The term “guide sequence” herein also includes the corresponding DNA or DNA encoding the RNA guide sequence.

The expression “RNA corresponding to the isolated guide sequence” includes RNA encoded by DNA guide sequences. The term “tracr mate sequence” may also be used interchangeably with the term “direct repeat(s)”.

The term “sgRNA library” and “gRNA” library may be used interchangeably. They can comprise single guide RNAs or guide sequences.

“Complementarity” refers to the ability of a nucleic acid to form hydrogen bond(s) with another nucleic acid sequence by either traditional Watson-Crick base pairing or other non-traditional types.

A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100%) complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refers to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y.

A sequence capable of hybridizing with a given sequence is referred to as the “complement” of the given sequence.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

Several aspects of the invention relate to vector systems comprising one or more vectors, or vectors as such. Vectors can be designed for expression of CRISPR transcripts (e.g. nucleic acid transcripts, proteins, or enzymes) in prokaryotic or eukaryotic cells. Alternatively, the recombinant expression vector can be transcribed and translated in vitro, for example the lentiviral vectors encompassed in aspects of the invention may comprise a U6 RNA pol III promoter.

Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses). Viral vectors also include polynucleotides earned by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and HI promoters. Examples ofpol II promoters include, but are not limited to, the retroviral Rous sarcoma virus (R.SV) LTR. promoter (optionally with the RSV enhancer), the cytomegalovirus (CMV) promoter (optionally with the CMV enhancer) [see, e.g., Boshart et al, Cell, 41:521-530 (1985)], the SV4G promoter, the dihydro folate reductase promoter, the β-actin promoter, the phosphoglycerol kinase (PGK) promoter. Also encompassed by the term “regulatory element” are enhancer elements, such as WPRE; CMV enhancers; the R-U5′ segment in LTR of HTLV-I (Mol. Cell. Biol, Vol. 8(1), p. 466-472, 1988); SV40 enhancer; and the intron sequence between exons 2 and 3 of rabbit 3-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Advantageous vectors include lentiviruses, adenoviruses and adeno-associated viruses, and types of such vectors can also be selected for targeting particular types of cells. In aspects on the invention the vectors may include but are not limited to packaged vectors. In other aspects of the invention a population of cells or host cells may be transduced with a vector with a low multiplicity of infection (MOI). As used herein the MOI is the ratio of infectious agents (e.g. phage or virus) to infection targets (e.g. cell). For example, when referring to a group of cells inoculated with infectious virus particles, the multiplicity of infection or MOI is the ratio of the number of infectious virus particles to the number of target cells present in a defined space (e.g. a well in a plate). In embodiments of the invention the cells are transduced with an MOI of 0.3-0.75 or 0.3-0.5; in preferred embodiments, the MOI has a value close to 0.4 and in more preferred embodiments the MOI is 0.3. In aspects of the invention the vector library of the invention may be applied to a well of a plate to attain a transduction efficiency of at least 20%, 30%, 40%, 50%, 60%, 70%, or 80%. In a preferred embodiment the transduction efficiency is approximately 30% wherein it may be approximately 370-400 cells per lentiCRISPR construct. In a more preferred embodiment, it may be 400 cells per lentiCRISPR construct.

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. Bacterid., 169:5429-5433 [1987]; and Nakata et al, J. Bacterid., 171:3553-3556 [1989]), and associated genes. Similar interspersed SSRs have been identified in Haloferax mediterranei, Streptococcus pyogenes, Anabaena, and Mycobacterium, tuberculosis (See, Groenen et al., Mol. Microbiol, 10: 1057-1065 [1993]; Hoe et al., Emerg. Infect. Dis., 5:254-263 [1999]; Masepohl et al., Biochim. Biophys. Acta 1307:26-30 [1996]; and Mojica et al., Mol. Microbiol, 17:85-93 [1995]). The CRISPR loci typically differ from other SSRs by the structure of the repeats, which have been termed short regularly spaced repeats (SRSRs) (Janssen et al., OMICS J. Integ. Biol, 6:23-33 [2002]; and Mojica et al, Mol. Microbiol, 36:244-246 [2000]). In general, the repeats are short elements that occur in clusters that are regularly spaced by unique intervening sequences with a substantially constant length (Mojica et al, [2000], supra). Although the repeat sequences are highly conserved between strains, the number of interspersed repeats and the sequences of the spacer regions typically differ from strain to strain (van Embden et al, J, Bacteriol, 182:2393-2401 [2000]). CRISPR loci have been identified in more than 40 prokaryotes (See e.g., Jansen et al, Mol. Microbiol, 43; 1565-1575 [2002]; and Mojica et al, [2005]) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium., Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In aspects of the invention functional genomics screens allow for discovery of novel human and mammalian therapeutic applications, including the discovery of novel drugs, for, e.g., treatment of genetic diseases, cancer, fungal, protozoal, bacterial, and viral infection, ischemia, vascular disease, arthritis, immunological disorders, etc. As used herein assay systems may be used for a readout of cell state or changes in phenotype include, e.g., transformation assays, e.g., changes in proliferation, anchorage dependence, growth factor dependence, foci formation, growth in soft agar, tumor proliferation in nude mice, and tumor vascularization in nude mice; apoptosis assays, e.g., DNA laddering and cell death, expression of genes involved in apoptosis; signal transduction assays, e.g., changes in intracellular calcium, cAMP, cGMP changes in hormone and neurotransmitter release; receptor assays, e.g., estrogen receptor and cell growth; growth factor assays, e.g., EPO, hypoxia and erythrocyte colony forming units assays; enzyme product assays, e.g., FAD-2 induced oil desaturation; transcription assays, e.g., reporter gene assays; and protein production assays, e.g., VEGF ELISAs.

Aspects of the invention relate to modulation of gene expression and modulation can be assayed by determining any parameter that is indirectly or directly affected by the expression of the target candidate gene. Such parameters include, e.g., changes in RNA or protein levels, changes in protein activity, changes in product levels, changes in downstream gene expression, changes in reporter gene transcription (luciferase, CAT, bet.-galactosidase, beta-glucuronidase, GFP (see, e.g., Mistili & Spector, Nature Biotechnology 15:961-964 (1997)); changes in signal transduction, phosphorylation and dephosphorylation, receptor-ligand interactions, second messenger concentrations (e.g., cGMP, cAMP, IP3), cell growth, and neovascularization, etc., as described herein. These assays can be in vitro, in vivo, and ex vivo. Such functional effects can be measured by any means known to those skilled in the art, e.g., measurement of RNA or protein levels, measurement of RNA stability, identification of downstream or reporter gene expression, e.g., via chemiluminescence, fluorescence, calorimetric reactions, antibody binding, inducible markers, ligand binding assays; changes in intracellular second messengers such as cGMP and inositol triphosphate (IP3); changes in intracellular calcium levels; cytokine release, and the like, as described herein.

To determine the level of gene expression modulated by the CRISPR-Cas system, cells contacted with the CRISPR-Cas system are compared to control cells, e.g., without the CRISPR-Cas system or with a non-specific CRISPR-Cas system, to examine the extent of inhibition or activation. Control samples may be assigned a relative gene expression activity value of 100%. Modulation/inhibition of gene expression is achieved when the gene expression activity value relative to the control is about 80%, preferably 50% (i.e., 0.5 times the activity of the control), more preferably 25%, more preferably 5-0%. Modulation/activation of gene expression is achieved when the gene expression activity value relative to the control is 110%, more preferably 150%) (i.e., 1.5 times the activity of the control), more preferably 200-500%, more preferably 1000-2000% or more.

In general, “CRISPR system”, “CRISPR-Cas” or the “CRISPR-Cas system” may refer collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRJSPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template, in an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”), in some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Cs 12), Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has UNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needieman-Wimsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

The term “variant” as used herein refers to a sequence, polypeptide or protein having substantial or significant sequence identity or similarity to a parent sequence, polypeptide or protein. Said variant are functional, i.e. retain the biological activity of the sequence, polypeptide or protein of which it is a variant. In reference to the parent sequence, polypeptide or protein, the functional variant can, for instance, be at least about 30%, 50%, 75%, 80%, 90%, 95%, 96%, 97%, 98%, 99% or more identical in amino acid sequence to the parent sequence, polypeptide, or protein.

The functional variant can, for example, comprise the amino acid sequence of the parent sequence, polypeptide, or protein with at least one conservative amino acid substitution. Conservative amino acid substitutions are known in the art, and include amino acid substitutions in which one amino acid having certain physical and/or chemical properties is exchanged for another amino acid that has the same chemical or physical properties.

Alternatively or additionally, the functional variants can comprise the amino acid sequence of the parent sequence, polypeptide, or protein with at least one non-conservative amino acid substitution.

In this case, it is preferable for the non-conservative amino acid substitution to not interfere with or inhibit the biological activity of the functional variant. Preferably, the non-conservative amino acid substitution enhances the biological activity of the functional variant, such that the biological activity of the functional variant is increased as compared to the parent sequence, polypeptide, or protein.

Variants also comprises functional fragment of the parent sequence, polypeptide, or protein and can comprise, for instance, about 10%, 25%, 30%, 50%, 68%, 80%, 90%, 95%, or more, of the parent sequence, polypeptide, or protein.

As used herein, the term “orthologues” refers to proteins or corresponding sequences in different species.

The invention will be illustrated by means of non-limiting examples in reference to the following figures.

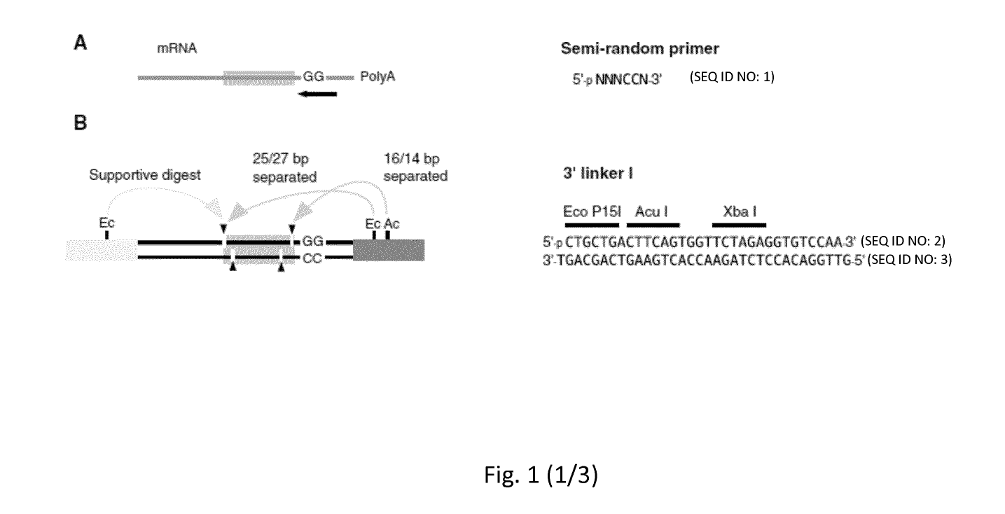

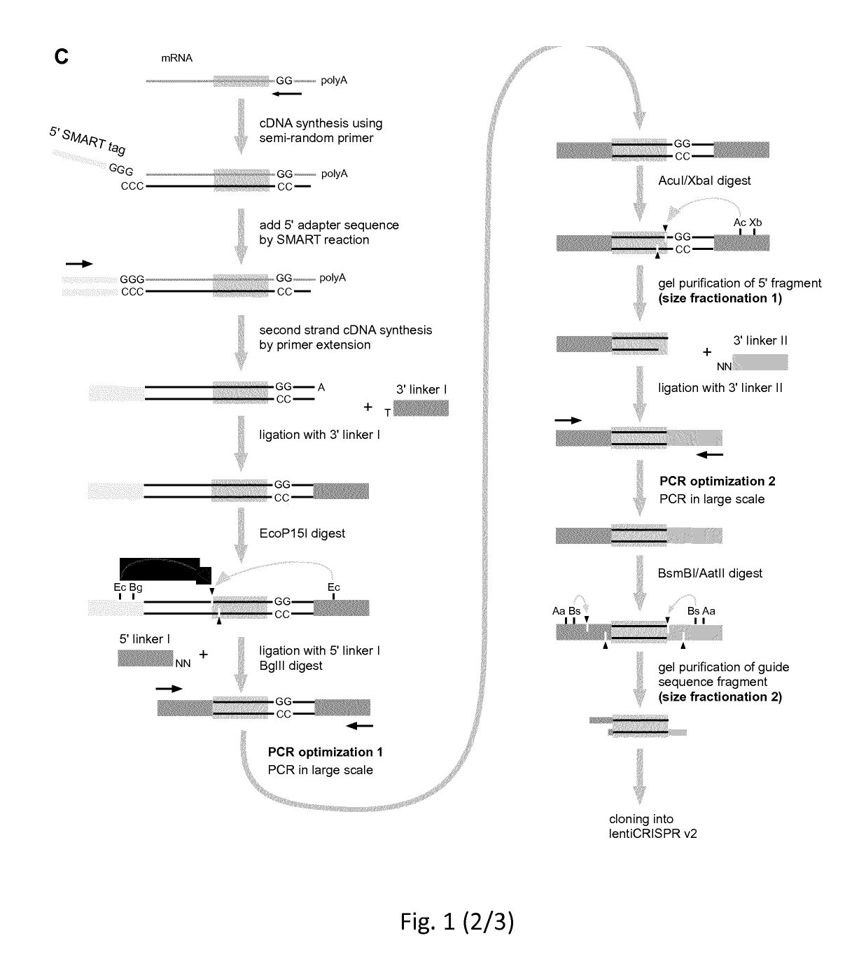

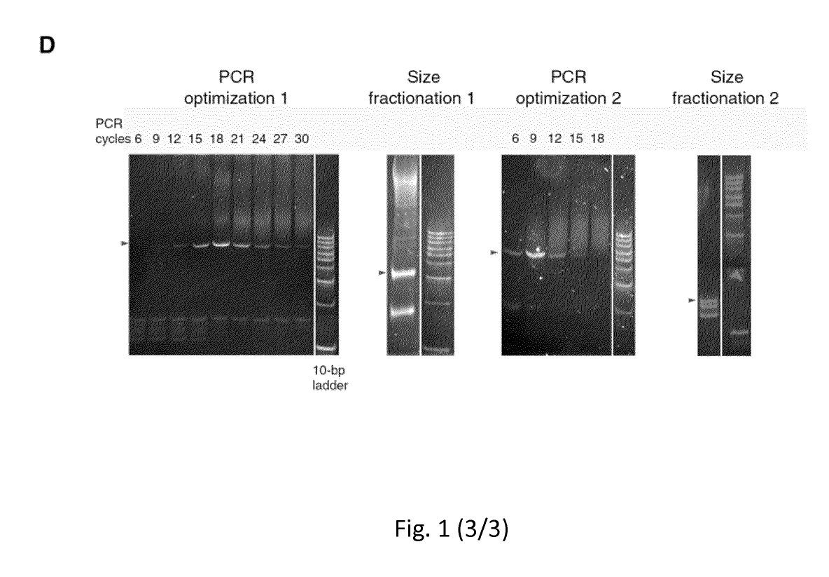

FIG. 1 gRNA library construction using a semi-random primer. A. Semi-random primer. B. Type III and IIS restriction sites to cut out the 20-bp guide sequence. Ec, EcoP15I; Ac, AcuI. C. Scheme of gRNA library construction. Bg, BglII; Xb, XbaI; Bs, BsmBI; Aa, AatII. D. Short-range PCR for PCR cycle optimization and size fractionation of the guide sequence. PCR products were run on 20% polyacrylamide gels. A 10-bp ladder was used as the size marker. Bands of the expected sizes are marked by triangles.

FIG. 2 Guide sequences in the gRNA library. (A) Mass sequencing of the gRNA library. (B) An example of sequencing for 12 random clones. (C) An example of the BLAST search analysis of a guide sequence. The first guide sequence clone in FIG. 2A is shown as an example. A 20-bp guide sequence (first frame) is accompanied by a protospacer adjacent motif (PAM; second frame). (D) Three different guide sequences derived from the same gene, the immunoglobulin (Ig) heavy chain Cμ gene. (E) Features of the gRNA library. Percentages in the PAM graph were calculated among the guide sequences where their origins were identified. “Others” in the gRNA-candidates graph indicates the sum of guide sequences of rRNA and PAM (−) mRNA.

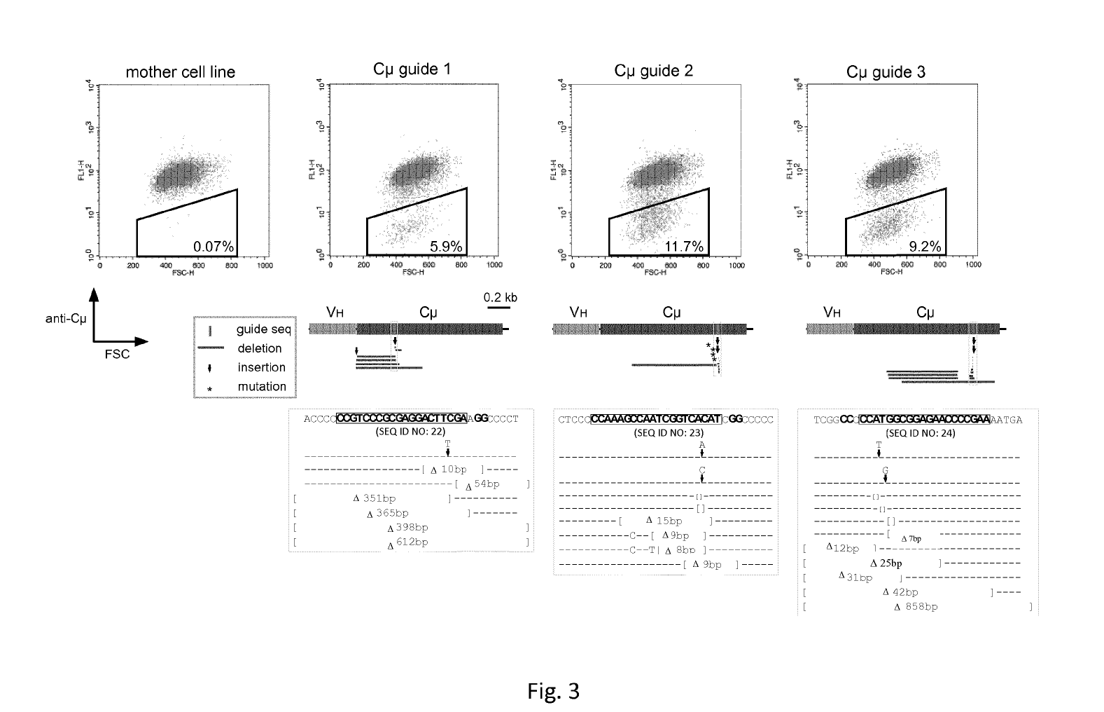

FIG. 3 Functional validation of guide sequences. Three lentivirus clones specific to Cμ (Cμ guides 1, 2, and 3 in FIG. 2d) were transduced into the AID^{-/-} cell surface IgM (sIgM) (+) DT40 cell line. FACS profiles two weeks after transduction are shown with the sIgM (−) gatings, which were used for FACS sorting (upper panels). The cDNA of the IgM gene from the sorted sIgM (−) cells is mapped together with the position of guide sequences, insertions, deletions, and mutations (lower panels). Detailed cDNA sequences around the guide sequences are shown below.

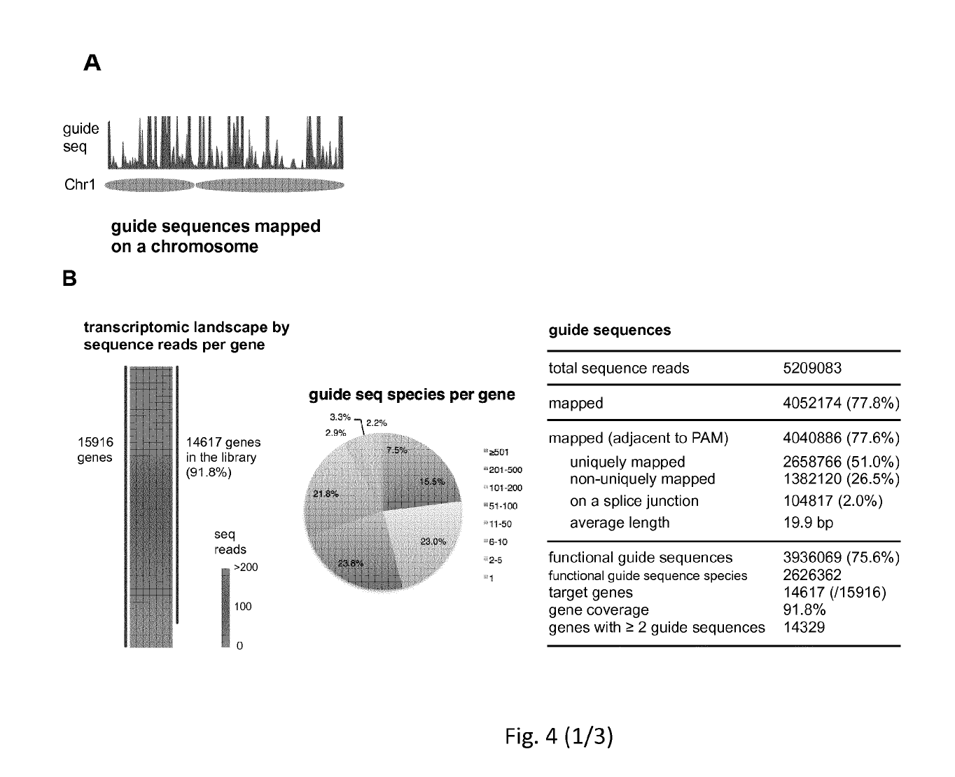

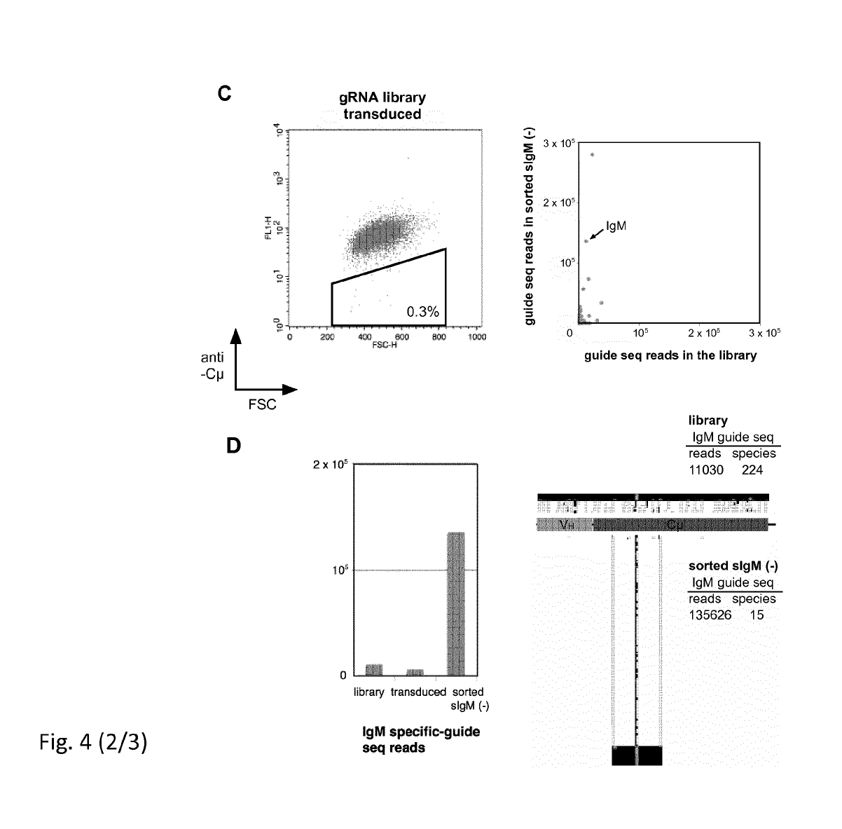

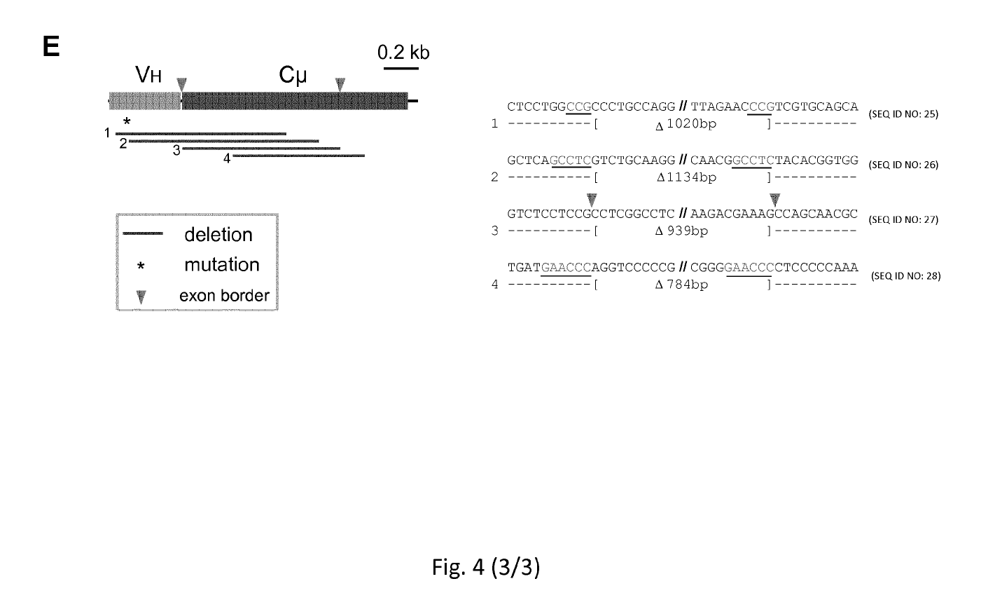

FIG. 4 Characterization and functional validation of the gRNA library. (A) Distribution of guide sequences on a chromosome. (B) Diversity of the gRNA library. Sequence reads per gene reflecting the transcriptomic landscape of the guide sequences (heat map; shown with a scale bar). Guide sequence species per gene (circle graph). (C) Lentiviral transduction of gRNA library. A FACS profile two weeks after transduction is shown with the sIgM (−) gating, which was used for FACS sorting (left panel). The graph shows the total sequence reads in the library versus those in the sorted sIgM (−) (right panel). Each dot represents a different gene. (D) IgM-specific guide sequences. Sequence reads specific to IgM (graph). Guide sequences mapped on IgM cDNA (map). (E) Deletions in the IgM cDNA in sorted sIgM (−). The cDNA of the IgM gene from sorted sIgM (−) cells is shown with the position of guide sequences, deletions, mutations, and exon borders (left panel). The detailed sequences around breakpoints are shown in the right panel. Micro-homologies in the reference sequences are underlined.

### EXAMPLE

Methods

Preparation of RNA

Total RNA was prepared from DT40^{Cre1} cells ^{(11, 12)} using TRIzol reagent (Invitrogen). Poly(A) RNA was prepared from DT40^{Cre1} total RNA using an Oligotex mRNA Mini Kit (Qiagen). To enrich mRNA, hybridization of poly(A)+ RNA and washing with buffer OBB (from the Oligotex kit) were repeated twice, according to the stringent wash protocol from the manufacturer's recommendations.

Oligonucleotides

The following oligonucleotides were used:

Semi-random primer

(SEQ ID NO: 1)

p NNNCCN
5′ SMART (switching mechanism at RNA transcript)
tag

(SEQ ID NO: 29)

TGGTCAAGCTTCAGCAGATCTACACGGACGTCGCrGrGrG
5′ SMART PCR primer

(SEQ ID NO: 30)

TGGTCAAGCTTCAGCAGATCTACACG
3′ linker I forward

(SEQ ID NO: 31)

p CTGCTGACTTCAGTGGTTCTAGAGGTGTCCAA
3′ linker I reverse

(SEQ ID NO: 32)

GTTGGACACCTCTAGAACCACTGAAGTCAGCAGT
5′ linker I forward

(SEQ ID NO: 33)

GCATATAAGCTTGACGTCTCTCACCG
5′ linker I reverse

(SEQ ID NO: 34)

p NNCGGTGAGAGACGTCAAGCTTATATGC
3′ linker II forward

(SEQ ID NO: 35)

p GTTTGGAGACGTCTTCTAGATCAGCG
3′ linker II reverse

(SEQ ID NO: 36)

CGCTGATCTAGAAGACGTCTCCAAACNN
3′ linker I PCR primer

(SEQ ID NO: 37)

GTTGGACACCTCTAGAACCACTGAAGTCAGCAGTNNNCC
3′ linker II PCR primer

(SEQ ID NO: 38)

CGCTGATCTAGAAGACGTCTCCAAAC
Sequencing primer

(SEQ ID NO: 39)

TTTTCGGGTTTATTACAGGGACAGCAG
lentiCRISPR forward

(SEQ ID NO: 40)

CTTGGCTTTATATATCTTGTGGAAAGGACG
lentiCRISPR reverse

(SEQ ID NO: 41)

CGGACTAGCCTTATTTTAACTTGCTATTTCTAG
universal forward

(SEQ ID NO: 42)

AGCGGATAACAATTTCACACAGGA
universal reverse

(SEQ ID NO: 43)

CGCCAGGGTTTTCCCAGTCACGAC
Ig heavy chain 1

(SEQ ID NO: 44)

CCGCAACCAAGCTTATGAGCCCACTCGTCTCCTCCCTCC
Ig heavy chain 2

(SEQ ID NO: 45)

CGTCCATCTAGAATGGACATCTGCTCTTTAATCCCAATCGAG
Ig heavy chain 3

(SEQ ID NO: 46)

GCTGAACAACCTCAGGGCTGAGGACACC
Ig heavy chain 4

(SEQ ID NO: 47)

AGCAACGCCCGCCCCCCATCCGTCTACGTCTT

Linker Preparation

The following reagents were combined in a 1.5 ml microcentrifuge tube: 10 μl of 100 μM linker forward oligo, 10 μl of 100 μM linker reverse oligo, and 2.2 μl of 10×T4 DNA ligase buffer (NEB). The tubes were placed in a water bath containing 2 l of boiled water and were incubated as the water cooled naturally. The annealed oligos were diluted with 77.8 μl of TE buffer (pH 8.0) and used as 10 μM linkers.

gRNA Library Construction

(1) First-Strand cDNA Synthesis

The following reagents were combined in a 0.2 ml PCR tube: 200 ng of DT40^{Cre1} poly(A) RNA, 0.6 μl of 25 μM semi-random primer, and RNase-free water in a 4.75 μl volume. The tube was incubated at 72° C. in a hot-lid thermal cycler for 3 min, cooled on ice for 2 min, and further incubated at 25° C. for 10 min. The temperature was then increased to 42° C. and a 5.25 μl mixture containing the following reagents was added: 0.5 μl of 25 μM 5′ SMART tag, 2 μl of 5× SMART Scribe buffer, 0.25 μl of 100 mM DTT, 1 μl of 10 mM dNTP Mix, 0.5 μl of RNaseOUT (Invitrogen), and 1 μl SMART Scribe Reverse Transcriptase (100 U) (Clontech). The first-strand cDNA reaction mixture was incubated at 42° C. for 90 min and then at 68° C. for 10 min. To degrade RNA, 1 μl of RNase H (Invitrogen) was added to the mixture and the mixture was incubated at 37° C. for 20 min.

(2) Double-Stranded (Ds) cDNA Synthesis by Primer Extension

Eleven μl of prepared first-strand poly(A) cDNA was mixed with 74 μl of milliQ water, 10 μl of 10× Advantage 2 PCR Buffer, 2 μl of 10 mM dNTP mix, 1 μl of 25 μM 5′ SMART PCR primer, and 2 μl of 50× Advantage 2 polymerase mix (Clontech). A 100 μl volume of the reaction mixture for primer extension was incubated at 95° C. for 1 min, 68° C. for 20 min, and then 70° C. for 10 min. The prepared ds cDNA was purified using a QIAquick PCR Purification Kit (Qiagen) and was eluted with 40 μl of TE buffer (pH 8.0).

(3) 3′ Linker I Ligation

DT40^{Cre1} ds poly(A) cDNA was mixed with 0.5 μl of 10 μM 3′ linker I and 1 μl of Quick T4 DNA ligase (New England Biolabs; NEB) in 1× Quick ligation buffer. The ligation reaction mixture was incubated at room temperature for 15 min, then purified using a QIAquick PCR Purification Kit, and eluted with 80 μl of TE buffer.

(4) EcoP15I Digestion

The 3′ linker I-ligated DNA was digested with 1 μl EcoP15I (10 U/μl, NEB) in 1× NEBuffer 3.1 containing 1×ATP in a 100 μl volume at 37° C. overnight. The EcoP15I-digested DNA was purified using a QIAquick PCR Purification Kit and eluted with 40 μl of TE buffer.

(5) 5′ Linker I Ligation and BglII Digestion

The digested DNA was mixed with 0.5 μl of 10 μM 5′ linker I and 1 μl of Quick T4 DNA ligase (NEB) in 1× Quick ligation buffer. The ligation reaction mixture was incubated at room temperature for 15 min, purified using a QIAquick PCR Purification Kit, and eluted with 80 μl of TE buffer. The DNA was further digested with 1 μl of BglII (10 U/μl, NEB) in 1× NEBuffer 3.1 in a 100 μl volume at 37° C. for 3 h. The EcoP15/BglII-digested DNA was purified using a QIAquick PCR Purification Kit and eluted with 50 μl of TE buffer.

(6) First PCR Optimization

To determine the optimal number of PCR cycles, a 0.2 ml PCR tube was prepared containing 5 μl of the ds cDNA ligated with 5′ linker I/3′ linker I, 0.5 μl of 25 μM 5′ linker I forward primer, 0.5 μl of 25 μM 3′ linker I PCR primer, 5 μl of 1× Advantage 2 PCR buffer, 1 μl of 10 mM dNTP mix, 1 μl of 50× Advantage 2 Polymerase mix, and milliQ water in a 50 μl volume. PCR was carried out with the following cycling parameters: 6 cycles of 98° C. for 10 s and 68° C. for 10 s. After the 6 cycles, 5 μl of the reaction were transferred to a clean microcentrifuge tube. The rest of the PCR reaction mixture underwent 3 additional cycles of 98° C. for 10 s and 68° C. for 10 s. After these additional 3 cycles, 5 μl were transferred to a clean microcentrifuge tube. In the same way, additional PCR was repeated until reaching 30 total cycles. Thus, a series of PCR reactions of 6, 9, 12, 15, 18, 21, 24, 27, and 30 cycles was prepared and analyzed by 20% polyacrylamide gel electrophoresis to compare the band patterns. The optimal number of PCR cycles was determined as the minimal number of PCR cycles yielding the greatest quantity of the 84-bp product (typically around 17 cycles). Two 50-μl PCR reactions were repeated with the optimal number of PCR cycles. The PCR product was purified using a QIAquick PCR Purification Kit and eluted with 50 μl of TE buffer.

(7) AcuI/XbaI Digestion

The PCR product was digested with 2 μl of AcuI (5 U/μl, NEB) and 2 μl of XbaI (20 U/μl, NEB) in 1× CutSmart Buffer containing 40 μM S-adenosylmethionine (SAM) in a 60 μl volume at 37° C. overnight. The AcuI/XbaI-digested DNA was run on a 20% polyacrylamide gel. The 45-bp fragment was cut out of the gel, purified by the crush and soak procedure, and dissolved into 20 μl of TE buffer.

(8) 3′ Linker II Ligation

The digested DNA was mixed with 2 μl of 10 μM 3′ linker II and 1 μl of Quick T4 DNA ligase (NEB) in 1× Quick ligation buffer. The ligation reaction mixture was incubated at room temperature for 15 min, purified using a QIAquick PCR Purification Kit, and eluted with 100 μl of TE buffer.

(9) Second PCR Optimization

To determine the optimal number of PCR cycles, a 0.2 ml PCR tube was prepared, containing 5 μl of the ds cDNA ligated with 5′ linker I/3′ linker II, 0.5 μl of 25 μM 5′ linker I forward primer, 0.5 μl of 25 μM 3′ linker II PCR primer, 5 μl of 1× Advantage 2 PCR buffer, 1 μl of 10 mM dNTP mix, 1 μl of 50× Advantage 2 Polymerase mix, and milliQ water in a 50 μl volume. PCR was carried out with the following cycling parameters: 6 cycles of 98° C. for 10 s and 68° C. for 10 s. After the 6 cycles, 5 μl of the reaction were transferred to a clean microcentrifuge tube. The rest of the PCR reaction mixture underwent an additional 3 cycles of 98° C. for 10 s and 68° C. for 10 s. After these additional 3 cycles, 5 μl of the reaction were transferred to a clean microcentrifuge tube. In the same way, additional PCR cycles were repeated until 18 total cycles were reached. Thus, a series of PCR reactions of 6, 9, 12, 15, and 18 cycles was prepared and analyzed by 20% polyacrylamide gel electrophoresis to compare the band patterns. The optimal number of PCR cycles was determined as the minimal number of PCR cycles yielding the greatest quantity of the 72-bp product (typically around 9 cycles). Five PCR reactions, each containing 50 μl, were repeated with the optimal number of PCR cycles. The PCR product was purified using a QIAquick PCR Purification Kit and eluted with 100 μl of TE buffer.

(10) BsmBI/AatII Digestion

The PCR product was digested with 10 μl of BsmBI (10 U/μl, NEB) in 1× NEBuffer 3.1 in a 100 μl volume at 55° C. for 6 h, and then 5 μl of AatII (20 U/μl, NEB) were added to the solution, which was left at 37° C. overnight. The BsmBI/AatII digested DNA was run on a 20% polyacrylamide gel. Typically, 3 bands, corresponding to 25, 24, and 23 bp, were visible. The 25-bp fragment was cut out of the gel, purified by the crush and soak procedure, and dissolved into 50 μl of TE buffer. The concentration of the purified DNA was measured by a Qubit dsDNA HS Assay Kit (Life Technologies).

(11) Cloning

The lenti CRISPR ver. 2 (lentiCRISPR v2) ⁽¹⁵⁾ (Addgene) was digested with BsmBI, treated with calf intestine phosphatase, extracted with phenol/chloroform, and purified by ethanol precipitation. Five ng of the purified 25-bp guide sequence fragment was mixed with 3 μg of lentiCRISPR v2 and 1 μl of Quick T4 DNA ligase (NEB) in 1× Quick ligation buffer in a 40 μl volume. The ligation reaction mixture was incubated at room temperature for 15 min and then purified by ethanol precipitation. The prepared gRNA library was electroporated into STBL4 electro-competent cells (Invitrogen) using the following electroporator conditions: 1200 V, 25 ρF, and 200Ω.

Sequencing and Sequence Analysis

Plasmid DNA was purified using a Wizard Plus SV Minipreps DNA Purification System (Promega) from 236 of the randomly-selected clones from the gRNA library, in accordance with the manufacturer's protocol. The guide sequence clones were sequenced with the sequencing primer using a model 373 automated DNA sequencer (Applied Biosystems). The cloned guide sequences were compared with the GenBank database using BLAST.

Optional Steps to Avoid Background Noise in the gRNA Library

During setup of the methodology for gRNA library construction, rRNA contamination was observed in poly(A) RNA purified using an oligo_{dT} column, and rRNA-originated guide sequences sometimes occupied 40-50% of the total original library. Since rRNA occupies more than 90% of intracellular RNA, generally speaking, it is hard to avoid having some rRNA contamination. The stringent wash protocol for poly(A) RNA purification successfully reduced the rRNA-derived guide sequences to around 10%. PCR artifacts amplifying the linker sequences were also observed during setup of the methodology. For this reason, the linker sequence was designed with additional restriction sites, namely BglII for the 5′ SMART tag, XbaI for the 3′ linker I, and AatII for the 5′ linker I and 3′ linker II. By cutting with these additional restriction enzymes, it was possible to remove most of the PCR artifacts amplifying the linker sequences. The BsmBI restriction digest of the final PCR reaction generated the right size of DNA fragment (25 bp) in addition to one- or two-bp shorter, unexpected DNA fragments. These shorter DNA fragments were probably due to the inaccuracy of the cleavage position of the type III and type IIS restriction enzymes. After BsmBI cleavage, it was possible to minimize shorter DNA artifacts by carefully purifying the 25-bp fragment with a 20% polyacrylamide gel.

Lentiviral Vectors

lentiCRISPR v2 (15) was provided by from Feng Zhang (Addgene plasmid #52961). pCMV-VSV-G (25) was provided by Bob Weinberg (Addgene plasmid #8454). psPAX2 was provided by Didier Trono (Addgene plasmid #12260).

Lentiviral Packaging

To produce lentivirus, a T-225 flask of HEK293T cells was seeded at ˜40% confluence the day before transfection in D10 medium (DMEM supplemented with 10% fetal bovine serum). One hour prior to transfection, the medium was removed and 13 mL ofpre-warmed reduced serum OptiMEM medium (Life Technologies) was added to the flask. Transfection was performed using Lipofectamine 2000 (Life Technologies). Twenty μg of gRNA plasmid library, 10 μg of pCMV-VSV-G (25) (Addgene), and 15 μg of psPAX2 (Addgene) was mixed with 4 ml of OptiMEM (Life Technologies). One hundred μl of Lipofectamine 2000 was diluted in 4 ml of OptiMEM and this solution was, after 5 min, added to the mixture of DNA. The complete mixture was incubated for 20 min before being added to cells. After overnight incubation, the medium was changed to 30 ml of D10. After two days, the medium was removed and centrifuged at 3000 rpm at 4° C. for 10 min to pellet cell debris. The supernatant was filtered through a 0.45 μm low-protein-binding membrane (Millipore Steriflip HV/PVDF). The gRNA library virus was further enriched 100-fold by PEG precipitation.

Lentiviral vectors containing Cμ guide sequences were packaged as described above except for the following modifications. Five μg of Cμ guide-lentiviral vectors was used instead of 20 μg of the gRNA library. The experiment was done in a quarter-scale concerning solutions or culture medium without changing incubation times. 100-mm plates were used for lentiviral packaging instead of a T-225 flask. Cμ gRNA virus was directly used for transduction without enrichment by PEG precipitation.

Lentiviral Transduction

Cells were transduced with the gRNA library via spinfection. Briefly, 2×10⁶ cells per well were plated into a 12-well plate in DT40 culture medium supplemented with 8 μg/ml polybrene (Sigma). Each well received either 1 ml of Cμ gRNA virus or 100 μl of 100-fold enriched gRNA library virus along with a no-transduction control. The 12-well plate was centrifuged at 2,000 rpm for 2 h at 37° C. Cells were incubated overnight, transferred to culture flasks containing DT40 culture medium, and then selected with 1 μg/ml puromycin.

Sorting of sIgM (−) Population

The AID^{-/-} sIgM (+) cell line with or without lentiviral transduction was first stained with a monoclonal antibody to chicken Cμ (M1) (Southern Biotech) and then with polyclonal fluorescein isothiocyanate-conjugated goat antibodies to mouse IgG (Fab)₂ (Sigma). The sIgM (−) population was sorted using the FACSAria (BD Biosciences).

Cloning and Sequencing of the Ig Heavy Chain Gene

The sorted sIgM (−) cells were further expanded and used for total RNA and genomic DNA preparation. Total RNA was purified using TRIzol reagent (Invitrogen). Total RNA was reverse-transcribed using SuperScript III Reverse Transcriptase (Invitrogen) with oligo_{dT} primer according to the manufacturer's instructions. The IgM heavy chain gene was amplified from the total cDNA of the sorted sIgM (−) population with Ig heavy chain 1 and 2 primers. PCR was performed using Q5 Hot Start High-Fidelity DNA Polymerase (NEB) with the following cycling parameters: 30 s of initial incubation at 98° C., 35 cycles consisting of 10 s at 98° C. and 2 min at 72° C., and a final elongation step of 2 min at 72° C. The PCR product was purified by a QIAquick Gel Extraction Kit (Qiagen), digested with HindIII (NEB) and XbaI (NEB), and cloned into the pUC119 plasmid vector. Approximately 30 plasmid clones for each sorted sIgM (−) population were sequenced using universal forward, reverse, and Ig heavy chain 3 and 4 primers.

Deep Sequencing

Genomic DNA of the transduced cell library or sorted sIgM (−) cells was purified using an Easy-DNA Kit (Invitrogen). Either 100 ng of lentiviral plasmid library or 1 μg of genomic DNA were used as the PCR template. The guide sequences were amplified with lentiCRISPR forward and reverse primers using Advantage 2 Polymerase (Clontech). PCR was carried out with the following cycling parameters: 15 cycles of 98° C. for 10 s and 68° C. for 10 s for plasmid DNA, or 27 cycles of 98° C. for 10 s and 68° C. for 10 s for genomic DNA. The 100-bp PCR fragment containing the guide sequence was purified using a QIAquick Gel Extraction Kit (Qiagen). The deep sequencing library was prepared using a TruSeq Nano DNA Library Preparation Kit (Illumina), and deep sequenced using Miseq (Illumina).

Bioinformatics

FASTQ files demultiplexed by Illumina Miseq were analyzed using the CLC Genomics Workbench (Qiagen). Briefly, the sequence reads were trimmed to exclude vector backbone sequences and added with the PAM-sequence NGG. The sequence reads before or after adding NGG were aligned with the Ensemble chicken genome database (16) using the RNA seq analysis toolbox with the read mapping parameters optimized for comprehensive analysis. After alignment, duplicates were removed from the mapped sequence reads in order to identify different guide sequence species. Afterwards, the guide sequence reads and species per gene were calculated from the numbers of sequence reads mapped on the annotated genes. Since Ig genes were not annotated in the Ensemble database, the cDNA sequence of the IgM gene of the AID knockout DT40 cell line was used as a reference for the mapping of guide sequences specific to IgM.

Results

Strategy to Convert mRNA to Guide Sequences

A random primer is commonly used for cDNA synthesis. The present inventor found out that a semi-random primer containing a PAM-complementary sequence could be used as the cDNA synthesis primer instead of a random primer (FIG. 1a).

Type IIS or type III restriction enzymes cleave sequences separated from their recognition sequences. The type III restriction enzyme, EcoP15I, cleaves 25/27 bp away from its recognition site but requires a pair of inversely-oriented recognition sites for efficient cleavage⁽¹⁰⁾. The type IIS restriction enzyme, AcuI, cleaves 13/15 bp away from its recognition site. The present inventor now developed an approach that allows to cut out a 20-mer by carefully arranging the positions of these restriction sites (FIG. 1b).

gRNA Library Construction Via Molecular Biology Techniques

Using a semi-random primer (NCCNNN) that contained the PAM-complementary CCN, cDNA was reverse-transcribed from poly(A) RNA of the chicken B cell line DT40^{Cre1 (11, 12)} (FIG. 1c). At that time, the 5′ SMART tag sequence containing the EcoP15I site was added onto the 5′ side by the switching mechanism at RNA transcript (SMART) method¹³. The second strand of cDNA was synthesized by primer extension using a primer that annealed at the 5′ SMART tag sequence with Advantage 2 PCR polymerase, which generated A-overhang at the 3′ terminus. This A-overhang was ligated with 3′ linker I, which contains EcoP15I and AcuI sites for cutting out the guide sequence afterwards. The ds cDNA was digested with EcoP15I to remove the 5′ SMART tag sequence and was ligated with 5′ linker I that included a BsmBI site, a cloning site for the gRNA expression vector. The DNA was then digested with BglII to destroy the 5′ SMART tag backbone. The gRNA library at this stage was amplified by PCR. To determine the optimal number of PCR cycles, a titration between 6 and 30 cycles was performed (FIG. 1d; PCR optimization 1). The expected PCR product, approximately 80 bp, was visible after 12 cycles; however, as the number of cycles increased, a larger, non-specific appeared. In addition, unnecessary cycle number increases may reduce the complexity of the library. Thus, PCR amplification was repeated on a large scale using the optimal PCR cycle number of around 17 cycles. The PCR product was subsequently digested with AcuI and XbaI and examined using 20% polyacrylamide gel electrophoresis. The 45-bp fragment was purified (FIG. 1d; size fractionation 1), ligated with the 3′ linker II that included a BsmBI cloning site, and used for the next PCR.

To determine the optimal PCR cycle number, a titration between 6 and 18 PCR cycles was additionally performed (FIG. 1d; PCR optimization 2). PCR amplification was repeated on a large scale with the optimal number of 9 PCR cycles. The PCR product was then digested with BsmBI and AatII. The restriction digest generated the 25-bp fragment, as well as 24- and 23-bp fragments (FIG. 1d; size fractionation 2), which were likely generated due to the inaccurate breakpoints of the type IIS and type III restriction enzymes¹⁴; careful purification of the 25-bp fragment minimized the possible problems with those artifacts. The guide sequence insert library, generated as described above, was finally cloned into a BsmBI-digested lentiCRISPR v2¹⁵ vector and then electroporated into STBL4 electro-competent cells.

Guide Sequences in the gRNA Library

Plasmid DNA was purified from the generated gRNA library by maxiprep. Initially, the DNA was sequenced as a mixed plasmid population. A highly complexed and heterogeneous sequence was observed in the lentiCRISPR v2 cloning site between the U6 promoter and gRNA scaffold (FIG. 2a), indicating that: 1) no-insert clones are rare, 2) cloned guide sequences are highly complexed, and 3) the majority of guide sequences are 20 bp long. After re-transformation of the library in bacteria, a total of 236 bacterial clones were randomly picked and used for plasmid miniprep and sequencing.

As shown in the example of sequencing for 12 random clones (FIG. 2b), the cloned guide sequences were heterogeneous. These guide sequences were subsequently analyzed using NCBI's BLAST search. As shown in FIG. 2c, typically one gene was hit by each guide sequence. Importantly, a PAM was identified adjacent to the guide sequence. For more than three quarters of the guide sequences, the original genes from which those guides were generated were identified in the BLAST search. Most such guide sequences were derived from single genes.

Notably, three of the guide sequences among the 236 plasmid clones were derived from different positions adjacent to the PAMs on the immunoglobulin (Ig) heavy chain Cμ gene (FIG. 2d).

Thus, multiple guide sequences were generated from the same gene. Unexpectedly, the reversed-orientation guide sequences, like Cμ guide 3 (FIG. 2D), were also observed at a relatively low frequency (˜10%) (Table I). Most of these were, however, accompanied by a PAM (Table I). PAM-priming might have worked even from the first strand cDNA and not only from mRNA. These reversed guide sequences are expected to work in genome cleavage, contributing to the knockout library.

The cloning of the guide sequences was efficient (100%), and most guide sequences (89%) were 20 bp long (FIG. 2e, Table I).). While 66% of the insert sequences were derived from mRNA, 11% of the insert sequences were derived from rRNA and 23% were from unknown origins, possibly derived from unannotated genes (FIG. 2e). Importantly, 91% of the guide sequences with identified origins were accompanied by PAMs, which confirms that PAM-priming using the semi-random primer functioned as intended. In addition, PAMs were also found near of most of the remaining guide sequences (7%), but separated by 1 bp (FIG. 2e). This is most likely due to the inaccurate breakpoints of AcuI, since the length of those guide sequences was often 19 bp.

Functional Validation of Guide Sequences

Three guide sequences specific to Cμ (FIG. 2D) were further tested to functionally validate the guide sequences in the library. These lentiviral clones were transduced into the AID^{-/-} DT40 cell line, which constitutively expresses cell surface IgM (sIgM) due to the absence of immunoglobulin gene conversion (12). The Cμ guides 1, 2, and 3 generated 5.9%, 11.7%, and 9.2% sIgM (−) populations two weeks after transduction, as estimated by flow cytometry analysis (FIG. 3, upper panels), and these sIgM (−) populations were further isolated by FACS sorting. Since the Ig heavy chain genomic locus is poorly characterized and only the rearranged VDJ allele is transcribed, its cDNA, rather than its genomic locus, was analyzed by Sanger sequencing. Sequencing analysis of about 30 IgM cDNA-containing plasmid clones for each sorted sIgM (−) population clarified the insertions, deletions, and mutations on the locus (FIG. 3, lower panels). Most of the indels were focused around the guide sequences. Relatively large deletions observed on the cDNA sequence indicate that the clones in the library can sometimes cause even large functional deletions in the corresponding transcripts.

Deep Characterization of the gRNA Library

To characterize the complexity of the gRNA library, the library was deep-sequenced using Illumina Miseq and analyzed by a RNA seq protocol using the Ensemble chicken genome database (16) as a reference. For example, approximately 500,000 of the guide sequences were mapped to chromosome 1, suggesting robust generation of guide sequences from various loci in the genome. Although the Ensemble database includes 15,916 chicken genes, the number of annotated chicken genes appears to be at least 4,000 less than those in other established genetic model vertebrates such as humans, mice, and zebrafish (16). Among the 5,209,083 sequence reads, 4,052,174 reads (77.8%) were mapped to chicken genes, and most of those sequences were accompanied by PAM (FIG. 4B). Nevertheless, one quarter of the unmapped reads could be due to the relatively poor genetic annotation of the chicken genome, which again emphasizes the limitations of bioinformatics approaches for specific species. The average length of guide sequence reads was 19.9 bp. Although 2.0% of the guide sequences that mapped to exon/exon junctions appeared non-functional, 3,936,069 (75.6%) of the guide sequences, including 2,626,362 different guide sequences, were considered as functional. Guide sequences were generated even from genes with low expression levels, covering 91.8% of annotated genes (14,617/15,916) (FIG. 4B, heatmap). While two or more unique guide sequences were identified for 97.8% of those genes, more than 100 different guide sequence species were identified for 46.0% of genes (FIG. 4B, circle graph). Thus, the gRNA library appeared to have sufficient diversity for genetic screening.

Functional Validation of the gRNA Library

The transduction of the library into the AID^{-/-} DT40 cell line induced a significant sIgM (−) population (0.3%) (FIG. 4C, left) compared to the mother cell line (FIG. 3, left). This sIgM (−) population was further enriched 100-fold by FACS sorting, and their guide sequences were analyzed by deep sequencing. Unexpectedly, contaminated sIgM (+) cells appeared to expand more rapidly than sIgM (−) cells, possibly due to B-cell receptor signaling, leading to incomplete enrichment of sIgM (−) cells. Nevertheless, as IgM-specific guide sequences achieved the second-highest score of sequence reads in the sorted sIgM (−) population (FIG. 4C, right), IgM-specific guide sequences were obviously enriched after sIgM (−) sorting (FIG. 4D, left). While 224 of the unique guide sequences specific to IgM were identified in the plasmid library, a few such guide sequences were highly increased in the sorted sIgM (−) population (FIG. 4D, right). Sanger sequencing of 29 plasmid clones of the IgM cDNA from the sorted sIgM (−) population independently identified 4 deletions and 1 mutation (FIG. 4E). Three large deletions were likely generated by alternative non-homologous end joining via micro-homology, and one appeared to be generated by mis-splicing, possibly due to indels around splicing signals. Therefore, the library can be used to screen knockout clones when the proper screening method is available.

Taken together, a diverse and functional gRNA library was successfully generated using the described method. The generated gRNA library is a specialized short cDNA library and is, therefore, also useful as a customized gRNA library specific to organs or cell lines.

The present inventor generated a gRNA library for a higher eukaryotic transcriptome using molecular biology techniques. This is the first gRNA library created from mRNA and the first library created from a rather poorly genetically characterized species. The semi-random primer can potentially target any NGG on mRNA, generating a highly complex gRNA library that covers more than 90% of the annotated genes (FIG. 4B). Furthermore, the method described here could be applied to CRISPR systems in organisms other than S. pyogenes by customizing the semi-random primer.

Multiple guide sequences were efficiently generated from the same gene (FIGS. 2D, 4B, and 4D), like the native CRISPR system in bacteria (1); this is an important advantage of the developed method. Although each guide sequence may differ in genome cleavage efficiency for each target gene, relatively more efficient guide sequences for each gene are included in the library (FIG. 4D).

Because the gRNA library created here is on a B-cell transcriptomic scale rather than a genome scale, guide sequences will not be generated from non-transcribed genes. Guide sequences were more frequently generated from abundantly-transcribed mRNAs but less frequently generated from rare mRNAs (FIG. 4B). By combining the techniques of a normalized library, in which one normalizes the amount of mRNA for each gene, it is possible to increase the frequency of guide sequences generated from rare mRNA (19). If the promoters in the lentiCRISPR v2 for Cas9 or gRNA expression are replaced with optimal promoters for each cell type or species, this will further improve the transduction or knockout efficiency of the gRNA library.

Guide sequences can be generated not only from the coding sequence but also from the 5′ and 3′ untranslated regions (UTRs). Since gRNA from UTRs will not cause indels within the coding sequence, gRNAs are not usually designed on UTRs in order to knock out genes; however, because several key features, such as mRNA stability or translation control, are determined by regulatory sequences located in the UTRs, indels occurring in these areas can lead to the unexpected elucidation of the gene's function. In this regard, this method can be also usefully applied for species like human, whose large-scale gRNA libraries are already constructed (6-8). Indeed, it can be also useful to make personalized human gRNA libraries, which represent collections of single nucleotide polymorphisms from different exons. Such personalized human gRNA libraries could be used to study allelic variations and their phenotypes, leading to better characterisations of rare diseases.

Approximately 23% of the guide sequences were derived from unknown origins (FIG. 2E, 4B). These sequences may be, at least partly, derived from mRNA with insufficient genetic annotation. This is the greatest advantage of the developed method: the sum of these “unknown” sequences and PAM (+) mRNA cover 83% of the library and are expected guide sequence candidates available for genetic screening (FIG. 2E). Since this method is not based on bioinformatics, it is possible to create guide sequences even from unknown genetic information. Such a bioinformatics-independent approach is obviously advantageous for species with insufficient genetic analysis.

Some cell type-/species-specific biological properties may be driven by uncharacterized or unannotated genes. For example, the inventor suspects that such unknown genes may play a key role in Ig gene conversion (20) or hyper-targeted integration (21) in chicken B cells. Moreover, many “minor” organisms exist that have not been used as genetic models despite their unique biological characteristics, e.g., planaria with extraordinary regeneration ability (22), naked mole rats with cancer resistance (23), and red sea urchins with their 200-year lifespan (24). Knockout libraries can be important genetic tools to shed light on genetic backgrounds with unique biological properties. Using this technique, it is possible to create a gRNA library, even from species with poorly annotated genetic information; some “forgotten” species may be converted into attractive genetic models by this technology.

Typically, the cost to synthesize a huge number of oligos for construction of a gRNA library is enormous^{6,7}. Importantly, since only a limited number of oligos is required for the described approach, it is possible to reduce the cost of the library by more than 100-fold, compared to the method using the oligo library.

It is in fact difficult to bear the enormous technological or economic costs for such “forgotten” species. The described method is expected to overcome obstacles associated with the high cost of oligo-based gRNA library generation.

While the present inventor used poly(A) RNA as a starting material for this study, in principle it is also possible to start from DNA, if the method is modified properly. DNA polymerase, rather than a reverse transcriptase, is required for semi-random primer-primed DNA synthesis. Such a DNA synthesis will be performed by a non-thermostable DNA polymerase at low temperatures rather than PCR polymerase, since semi-random primers have low annealing temperatures. The 5′ tag sequence will be added by linker ligation to single-stranded DNA instead of the SMART method. In this way, it is also attractive to create a gRNA library from ready-made cDNA or cDNA libraries.

TABLE I
Guide Sequences

| | size | | | | | accession | |
|---|---|---|---|---|---|---|---|
| clone | (bp) | sequence | PAM | orientation | origin | number | gene |
| L9.2.2.100 | 20 | AACAGCACCCACCA | cgg | normal | mRNA | XM_415711 | PREDICTED: |
| | | CCACTG (SEQ ID | | | | | Gallus |
| | | NO: 48) | | | | | gallus |
| | | | | | | | POM121 |
| | | | | | | | transmembrane |
| | | | | | | | nucleoporin |
| | | | | | | | (POM121), |
| | | | | | | | partial |
| | | | | | | | mRNA. |
| L9.2.2.101 | 20 | CGTCGCCAAGACCT | cgg | normal | mRNA | CR387434 | Gallus gallus |
| | | CGAGGA(SEQ ID | | | | | finished |
| | | NO: 49) | | | | | cDNA, clone |
| | | | | | | | ChEST26e5 |
| L9.2.2.102 | 20 | TCGACGATGGCACG | cgg | normal | mRNA | NM_205337 | Gallus gallus |
| | | TCTGAT (SEQ ID | | | | | ribosomal |
| | | NO: 50) | | | | | protein L27 |
| | | | | | | | (RPL27), |
| | | | | | | | mRNA |
| L9.2.2.103 | 20 | GCGTTGTGGGGGAT | ggg | normal | mRNA | NM_001006475 | Gallus gallus |
| | | CGTCGG (SEQ ID | | | | | enhancer of |
| | | NO: 51) | | | | | rudimentary |
| | | | | | | | homolog |
| | | | | | | | (Drosophila) |
| | | | | | | | (ERH), |
| | | | | | | | mRNA |
| L9.2.2.104 | 20 | AAGGTGGTGCTGGT | cgg | normal | mRNA | NM_205337 | Gallus gallus |
| | | GCTCGC (SEQ ID | | | | | ribosomal |
| | | NO: 52) | | | | | protein L27 |
| | | | | | | | (RPL27), |
| | | | | | | | mRNA |
| L9.2.2.105 | 20 | CAGCACCGTGCTGA | ggg | normal | mRNA | XM_420326 | PREDICTED: |
| | | CATTTC (SEQ ID | | | | | Gallus |
| | | NO: 53) | | | | | gallus |
| | | | | | | | RAB39B, |
| | | | | | | | member RAS |
| | | | | | | | oncogene |
| | | | | | | | family |
| | | | | | | | (RAB39B), |
| | | | | | | | mRNA |
| L9.2.2.106 | 20 | GGCGCTGAGCAGCT | cgg | reverse | mRNA | NM_205406 | Gallus gallus |
| | | GTTCCT (SEQ ID | | | | | Y box |
| | | NO: 54) | | | | | binding |
| | | | | | | | protein 3 |
| | | | | | | | (YBX3), |
| | | | | | | | mRNA |
| L9.2.2.107 | 20 | GATAGGCACAATCTTTTCAC | | | | | |
| | | (SEQ ID NO: 55) | | | | | |
| L9.2.2.108 | 20 | ACCTCCAAGACCGG | cgg | normal | mRNA | AJ719748 | Gallus gallus |
| | | CAAGCA (SEQ ID | | | | | mRNA for |
| | | NO: 56) | | | | | hypothetical |
| | | | | | | | protein, clone |
| | | | | | | | 6a12 |
| L9.2.2.109 | 20 | CAGTCGCTCTTGGC | agg | normal | mRNA | XM_004943061 | PREDICTED: |
| | | ATTCTC (SEQ ID | | | | | Gallus |
| | | NO: 57) | | | | | gallus |
| | | | | | | | tetratricopeptide |
| | | | | | | | repeat, |
| | | | | | | | ankyrin |
| | | | | | | | repeat and |
| | | | | | | | coiled-coil |
| | | | | | | | containing 1 |
| | | | | | | | (TANC1), |
| | | | | | | | transcript |
| | | | | | | | variant X12, |
| | | | | | | | mRNA |
| L9.2.2.110 | 20 | GTCCGAGAAAGCAC | ggg | normal | mRNA | KP742951 | Gallus gallus |
| | | CTTCCA (SEQ ID | | | | | breed Rugao |
| | | NO: 58) | | | | | yellow |
| | | | | | | | chicken |
| | | | | | | | mitochondrion, |
| | | | | | | | complete |
| | | | | | | | genome |
| L9.2.2.111 | 20 | CCCTCTTATCCAGG | agg | normal | mRNA | NM_001012903 | Gallus gallus |
| | | ACCTAC (SEQ ID | | | | | annexin A11 |
| | | NO: 59) | | | | | (ANXA11), |
| | | | | | | | mRNA |
| L9.2.2.112 | 20 | TGCTGGGGTTCGTG | msmtch | normal | mRNA | KP742951 | Gallus gallus |
| | | TGTGTC (SEQ ID | | | | | breed Rugao |
| | | NO: 60) | | | | | yellow |
| | | | | | | | chicken |
| | | | | | | | mitochondrion, |
| | | | | | | | complete |
| | | | | | | | genome |
| L9.2.2.113 | 20 | GGGGTCGTCGAAGG | tgg | reverse | mRNA | NM_001001531 | Gallus gallus |
| | | ACACGG (SEQ ID | | | | | fused in |
| | | NO: 61) | | | | | sarcoma |
| | | | | | | | (FUS), |
| | | | | | | | mRNA |
| L9.2.2.114 | 20 | TATTAAATTAAAGCTCGTCC | | | | | |
| | | (SEQ ID NO: 62) | | | | | |
| L9.2.2.115 | 19 | CGAATACAGACCGT | cgg | normal | mRNA | AB556518 | Gallus gallus |
| | | GAAAG (SEQ ID | | | | | DNA, CENP- |
| | | NO: 63) | | | | | A associated |
| | | | | | | | sequence, |
| | | | | | | | partial |
| | | | | | | | sequence, |
| | | | | | | | clone: |
| | | | | | | | CAIP#220 |
| L9.2.2.116 | 20 | CCCGTGAAAATCCG | agg | normal | rRNA | FM165415 | Gallus gallus |
| | | GGGGAG (SEQ ID | | | | | 28S rRNA |
| | | NO: 64) | | | | | gene, clone |
| | | | | | | | GgLSU-1 |
| L9.2.2.117 | 19 | TGTATTTTGAAGAC | ggg | normal | mRNA | XM_418122 | PREDICTED: |
| | | AACGC (SEQ ID | | | | | Gallus |
| | | NO: 65) | | | | | gallus |
| | | | | | | | ribosomal |
| | | | | | | | protein L23 |
| | | | | | | | (RPL23), |
| | | | | | | | transcript |
| | | | | | | | variant X2, |
| | | | | | | | mRNA |
| L9.2.2.118 | 20 | CCCTGCTACGCTGC | cgg | normal | mRNA | NM_001282303 | Gallus gallus |
| | | CTTGTT(SEQ ID | | | | | cysteine-rich |
| | | NO: 66) | | | | | protein 1 |
| | | | | | | | (intestinal) |
| | | | | | | | (CRIP1), |
| | | | | | | | mRNA |
| L9.2.2.119 | 20 | CGCGATGAGGGAACTTCCGC | | | | | |
| | | (SEQ ID NO: 67) | | | | | |
| L9.2.2.120 | 20 | CAGTGCCTGCAGGA | tgg | reverse | mRNA | BX935029 | Gallus gallus |
| | | CCCTCC (SEQ ID | | | | | finished |
| | | NO: 68) | | | | | cDNA, clone |
| | | | | | | | ChEST304113 |
| L9.2.2.121 | 19 | CATGATTAAGAGGG | cgg | normal | rRNA | HQ873432 | Gallus gallus |
| | | ACGGC (SEQ ID | | | | | isolate ML48 |
| | | NO: 69) | | | | | 18S |
| | | | | | | | ribosomal |
| | | | | | | | RNA gene, |
| | | | | | | | partial |
| | | | | | | | sequence |
| L9.2.2.122 | 20 | CCGCAGCGACCGCA | ggg | normal | mRNA | XM_424134 | PREDICTED: |
| | | CGTCCC (SEQ ID | | | | | Gallus |
| | | NO: 70) | | | | | gallus |
| | | | | | | | ribosomal |
| | | | | | | | protein, large, |
| | | | | | | | P2 (RPLP2), |
| | | | | | | | mRNA |
| L9.2.2.123 | 20 | CGCGGTTTTCGTCCAATAAA | | | | | |
| | | (SEQ ID NO: 71) | | | | | |
| L9.2.2.124 | 19 | TCCTGTCCATGGCC | cgg | normal | mRNA | NM_001166326 | Gallus gallus |
| | | AACGC (SEQ ID | | | | | peptidylprolyl |
| | | NO: 72) | | | | | isomerase A |
| | | | | | | | (cyclophilin |
| | | | | | | | A) (PPIA), |
| | | | | | | | mRNA |
| L9.2.2.125 | 20 | GCCCGCAGCCGATC | cgg | normal | mRNA | NM_001030556 | Gallus gallus |
| | | CTCCGC (SEQ ID | | | | | cancer |
| | | NO: 73) | | | | | susceptibility |
| | | | | | | | candidate 4 |
| | | | | | | | (CASC4), |
| | | | | | | | mRNA |
| L9.2.2.126 | 19 | TCTGTATCTTCCTT | cgg | normal | mRNA | KP742951 | Gallus gallus |
| | | CACAT (SEQ ID | | | | | breed Rugao |
| | | NO: 74) | | | | | yellow |
| | | | | | | | chicken |
| | | | | | | | mitochondrion, |
| | | | | | | | complete |
| | | | | | | | genome |
| L9.2.2.127 | 20 | CGTCCACCTTTGCT | cgg | reverse | mRNA | XM_003643539 | PREDICTED: |
| | | TTCTTC (SEQ ID | | | | | Gallus |
| | | NO: 75) | | | | | gallus |
| | | | | | | | ribosomal |
| | | | | | | | protein L10- |
| | | | | | | | like |
| | | | | | | | (RPL10L), |
| | | | | | | | partial mRNA |
| L9.2.2.128 | 20 | CGAGGAATTCCCAG | cgg | normal | rRNA | HQ873432 | Gallus gallus |
| | | TAAGTG (SEQ ID | | | | | isolate ML48 |
| | | NO: 76) | | | | | 18S |
| | | | | | | | ribosomal |
| | | | | | | | RNA gene, |
| | | | | | | | partial |
| | | | | | | | sequence |
| L9.2.2.129 | 19 | TTTTGTTGGTTTTC | cgg | normal | rRNA | HQ873432 | Gallus gallus |
| | | GGAAA (SEQ ID | | | | | isolate ML48 |
| | | NO: 77) | | | | | 18S |
| | | | | | | | ribosomal |
| | | | | | | | RNA gene, |
| | | | | | | | partial |
| | | | | | | | sequence |
| L9.2.2.130 | 20 | GGCCCCCAAGATCG | tcgg (at | normal | mRNA | NM_001277679 | Gallus gallus |
| | | GACCGC (SEQ ID | +1 | | | | ribosomal |
| | | NO: 78) | | | | | protein L12 |
| | | | | | | | (RPL12), |
| | | | | | | | transcript |
| | | | | | | | variant 1, |
| | | | | | | | mRNA |
| L9.2.2.131 | 20 | CGGCTCCGGGACGG | agg | reverse | rRNA | DQ018756 | Gallus gallus |
| | | CTGGGA (SEQ ID | | | | | 28S |
| | | NO: 79) | | | | | ribosomal |
| | | | | | | | RNA gene, |
| | | | | | | | partial |
| | | | | | | | sequence |
| L9.2.2.132 | 20 | CGCAGCATTTATGGGCACAG | | | | | |
| | | (SEQ ID NO: 80) | | | | | |
| L9.2.2.133 | 20 | GGGATAAGGATTGG | ggg | | | | chr1: |
| | | CTCTAA (SEQ ID | | | | | 100348961-100348980 |
| | | NO: 81) | | | | | |
| L9.2.2.134 | 20 | TCCTAGAGCAAGGC | tgg | normal | mRNA | NM_001277139 | Gallus gallus |
| | | AAACGT (SEQ ID | | | | | M-phase |
| | | NO: 82) | | | | | phosphoprotein 6 |
| | | | | | | | (MPHOSPH6), |
| | | | | | | | mRNA |
| L9.2.2.135 | 20 | AACCCGACTCCGAG | cgg | normal | rRNA | DQ018756 | Gallus gallus |
| | | AAGCCC (SEQ ID | | | | | 28S |
| | | NO: 83) | | | | | ribosomal |
| | | | | | | | RNA gene, |
| | | | | | | | partial |
| | | | | | | | sequence |
| L9.2.2.136 | 20 | GCGCCGCCACCTTC | tgg | normal | mRNA | AF322051 | Gallus gallus |
| | | CGCAAC (SEQ ID | | | | | survivin |
| | | NO: 84) | | | | | mRNA, |
| | | | | | | | complete cds |
| L9.2.2.137 | 20 | GCGGGGAGCATGGCGGAGAG | | | | | |
| | | (SEQ ID NO: 85) | | | | | |
| L9.2.2.138 | 20 | GGGTGCGTTTGGGA | agg | normal | mRNA | L13234 | Gallus gallus |
| | | AGCCGC (SEQ ID | | | | | Jun-binding |
| | | NO: 86) | | | | | protein mRN, |
| | | | | | | | 3′ end |
| L9.2.2.139 | 20 | GGTTTTTTTCCTTAGCCAAG | | | | | |
| | | (SEQ ID NO: 87) | | | | | |
| L9.2.2.140 | 20 | CGCTTCCGGCGTCTTGCGCC | | | | | |
| | | (SEQ ID NO: 88) | | | | | |
| L9.2.2.141 | 20 | CCCCGCCTCCGCCTCCCCTC | | | | | |
| | | (SEQ ID NO: 89) | | | | | |
| L9.2.2.142 | 20 | CAGCCACAGGGCACAGTGAG | | | | | |
| | | (SEQ ID NO: 90) | | | | | |
| L9.2.2.143 | 20 | GCTGAAGAACATGAGCACGG | | | | | |
| | | (SEQ ID NO: 91) | | | | | |
| L9.2.2.144 | 20 | TCCCCGGCGCCGCT | ggg | reverse | rRNA | DQ018756 | Gallus gallus |
| | | CTCGGG (SEQ ID | | | | | 28S |
| | | NO: 92) | | | | | ribosomal |
| | | | | | | | RNA gene, |
| | | | | | | | partial |
| | | | | | | | sequence |
| L9.2.2.145 | 20 | AGCATACCAATCAG | cgg | normal | mRNA | KP742951 | Gallus gallus |
| | | CTACGC (SEQ ID | | | | | breed Rugao |
| | | NO: 93) | | | | | yellow |
| | | | | | | | chicken |
| | | | | | | | mitochondrion, |
| | | | | | | | complete |
| | | | | | | | genome |
| L9.2.2.146 | 20 | TCCTGTTGGCTGAG | ggg | normal | mRNA | NM_001006336 | Gallus gallus |
| | | GCTCGT (SEQ ID | | | | | major vault |
| | | NO: 94) | | | | | protein |
| | | | | | | | (MVP), |
| | | | | | | | mRNA |
| L9.2.2.147 | 20 | GGGGACGTAGGAGC | cgg | normal | mRNA | XM_003642222 | PREDICTED: |
| | | GTATCG (SEQ ID | | | | | Gallus |
| | | NO: 95) | | | | | gallus coiled- |
| | | | | | | | coil-helix- |
| | | | | | | | coiled-coil- |
| | | | | | | | helix domain- |
| | | | | | | | containing |
| | | | | | | | protein 2, |
| | | | | | | | mitochondrial- |
| | | | | | | | like |
| | | | | | | | (LOC416933), |
| | | | | | | | transcript |
| | | | | | | | variant X1, |
| | | | | | | | mRNA |
| L9.2.2.148 | 20 | AACCCAGGGGGCAA | agg | normal | mRNA | NM_001030831 | Gallus gallus |
| | | CTTTGA (SEQ ID | | | | | paraspeckle |
| | | NO: 96) | | | | | component 1 |
| | | | | | | | (PSPC1), |
| | | | | | | | mRNA |
| L9.2.2.149 | 20 | CTAACCCTCCTCTC | tgg | normal | mRNA | KP742951 | Gallus gallus |
| | | CCTAGC (SEQ ID | | | | | breed Rugao |
| | | NO: 97) | | | | | yellow |
| | | | | | | | chicken |
| | | | | | | | mitochondrion, |
| | | | | | | | complete |
| | | | | | | | genome |
| L9.2.2.150 | 20 | GGTCGGGCTGGGGC | cgg | normal | ? | | chr1: 100348931-100348950 |
| | | GCGAAG (SEQ ID | | | | | |
| | | NO: 98) | | | | | |
| L9.2.2.151 | 21 | TGGCACTTGCGGAA | ggg | reverse | mRNA | XM_003641377 | PREDICTED: |
| | | GCTTCCG (SEQ | | | | | Gallus |
| | | ID NO: 99) | | | | | gallus solute |
| | | | | | | | carrier family |
| | | | | | | | 43, member 3 |
| | | | | | | | (SLC43A3), |
| | | | | | | | transcript |
| | | | | | | | variant X1, |
| | | | | | | | mRNA |
| L9.2.2.152 | 20 | CCCACCCGTGTGACCCCGAA | | | | | |
| | | (SEQ ID NO: 100) | | | | | |
| L9.2.2.153 | 17 | GATTGAGATTTGGG | ctgg (at | normal | mRNA | NM_001006253 | Gallus gallus |
| | | TGT(SEQ ID NO: | +1) | | | | PEST |
| | | 101) | | | | | proteolytic |
| | | | | | | | signal |
| | | | | | | | containing |
| | | | | | | | nuclear |
| | | | | | | | protein |
| | | | | | | | (PCNP), |
| | | | | | | | mRNA |
| L9.2.2.154 | 20 | GGCAAACTCATGAA | agg | reverse | mRNA | XM_004934806 | PREDICTED: |
| | | AGCTGG(SEQ ID | | | | | Gallus |
| | | NO: 102) | | | | | gallus TBC1 |
| | | | | | | | domain |
| | | | | | | | family, |
| | | | | | | | member 22B |
| | | | | | | | (TBC1D22B), |
| | | | | | | | transcript |
| | | | | | | | variant X3, |
| | | | | | | | mRNA |
| L9.2.2.155 | 20 | GGGGCTGGACACAG | tgg | normal | mRNA | NM_001282277 | Gallus gallus |
| | | GGACGC(SEQ ID | | | | | ribosomal |
| | | NO: 103) | | | | | protein L17 |
| | | | | | | | (RPL17), |
| | | | | | | | mRNA |
| L9.2.2.156 | 20 | AGAAATGAAAATCG | cgg | normal | mRNA | XR_214191 | PREDICTED: |
| | | TTGTAG (SEQ ID | | | | | Gallus |
| | | NO: 104) | | | | | gallus |
| | | | | | | | uncharacterized |
| | | | | | | | LOC100857266 |
| | | | | | | | (LOC100857266), |
| | | | | | | | misc_RNA |
| L9.2.2.157 | 20 | CGGGGCGTGGGCAA | agg | normal | mRNA | NM_205461 | Gallus gallus |
| | | CCGCTG(SEQ ID | | | | | peptidylprolyl |
| | | NO: 105) | | | | | isomerase B |
| | | | | | | | (cyclophilin |
| | | | | | | | B) (PPIB), |
| | | | | | | | mRNA |
| L9.2.2.158 | 20 | TCCCGACGACCTCC | cgg | normal | mRNA | NM_001031597 | Gallus gallus |
| | | TGCAAC(SEQ ID | | | | | poly(A) |
| | | NO: 106) | | | | | binding |
| | | | | | | | protein, |
| | | | | | | | cytoplasmic 1 |
| | | | | | | | (PABPC1), |
| | | | | | | | mRNA |
| L9.2.2.159 | 20 | GTTGTGGCCATGGT | agg | normal | mRNA | NM_205047 | Gallus gallus |
| | | GTGGGA(SEQ ID | | | | | NME/NM23 |
| | | NO: 107) | | | | | nucleoside |
| | | | | | | | diphosphate |
| | | | | | | | kinase 2 |
| | | | | | | | (NME2), |
| | | | | | | | mRNA |
| L9.2.2.160 | 20 | CATGGCCCAGTTTTGCAAGT | | | | | |
| | | (SEQ ID NO: 108) | | | | | |
| L9.2.2.161 | 20 | GACAGGCGGTGCGG | ggg | normal | mRNA | NM_001012934 | Gallus gallus |
| | | GCTGGG(SEQ ID | | | | | proteasome |
| | | NO: 109) | | | | | (prosome, |
| | | | | | | | macropain) |
| | | | | | | | 26S subunit, |
| | | | | | | | non-ATPase, |
| | | | | | | | 2 (PSMD2), |
| | | | | | | | mRNA |
| L9.2.2.162 | 20 | TGAAGCTGGCACAC | agg | normal | mRNA | NM_001004379 | Gallus gallus |
| | | AAATAC(SEQ ID | | | | | ribosomal |
| | | NO: 110) | | | | | protein L7a |
| | | | | | | | (RPL7A), |
| | | | | | | | mRNA |
| L9.2.2.163 | 20 | TGCTTGTGCAGACC | cgg | normal | mRNA | NM_001006241 | Gallus gallus |
| | | AAGCGT(SEQ ID | | | | | ribosomal |
| | | NO: 111) | | | | | protein L3 |
| | | | | | | | (RPL3), |
| | | | | | | | mRNA |
| L9.2.2.164 | 20 | TGAGGGGAGCAGCA | agg | normal | mRNA | BX935029 | Gallus gallus |
| | | ATAAAA(SEQ ID | | | | | finished |
| | | NO: 112) | | | | | cDNA, clone |
| | | | | | | | ChEST304113 |
| L9.2.2.165 | 20 | TGGAGCCACCCCAG | cgg | normal | mRNA | NM_001277880 | Gallus gallus |
| | | GAAATT(SEQ ID | | | | | ribosomal |
| | | NO: 113) | | | | | protein S29 |
| | | | | | | | (RPS29), |
| | | | | | | | mRNA |
| L9.2.2.166 | 20 | CGTCCCCTCGCCAA | cgg | reverse | mRNA | NM_001012892 | Gallus gallus |
| | | TGACAC(SEQ ID | | | | | succinate- |
| | | NO: 114) | | | | | CoA ligase, |
| | | | | | | | alpha subunit |
| | | | | | | | (SUCLG1), |
| | | | | | | | mRNA |
| L9.2.2.167 | 20 | CGCCGGCCCCCCCCCAAACC | | | | | |
| | | (SEQ ID NO: 115) | | | | | |
| L9.2.2.168 | 20 | TGCCGATCCCTCCC | tgg | normal | mRNA | AJ606297 | Gallus gallus |
| | | GTCAAA(SEQ ID | | | | | mRNA for |
| | | NO: 116) | | | | | female- |
| | | | | | | | associated |
| | | | | | | | factor FAF |
| | | | | | | | (faf gene), |
| | | | | | | | clone FAF5 |
| L9.2.2.169 | 20 | GCAGCAGCGCTCCGTGCTCC | | | | | |
| | | (SEQ ID NO: 117) | | | | | |
| L9.2.2.170 | 19 | TCCACCCACACATA | ctgg (at | normal | mRNA | KP742951 | Gallus gallus |
| | | AACCC(SEQ ID | +1) | | | | breed Rugao |
| | | NO: 118) | | | | | yellow |
| | | | | | | | chicken |
| | | | | | | | mitochondrion, |
| | | | | | | | complete |
| | | | | | | | genome |
| L9.2.2.171 | 20 | TCCTCGGGACACACCCGCTC | | | | | |
| | | (SEQ ID NO: 119) | | | | | |
| L9.2.2.172 | 20 | TGCCAAATACGCAG | ggg | normal | mRNA | NM_205477 | Gallus gallus |
| | | AAGAGA(SEQ ID | | | | | myosin, |
| | | NO: 120) | | | | | heavy chain |
| | | | | | | | 9, non-muscle |
| | | | | | | | (MYH9), |
| | | | | | | | mRNA |
| L9.2.2.173 | 21 | AACAAAATGCTGTC | ggg | normal | mRNA | L13234 | Gallus gallus |
| | | CTGCGCC(SEQ ID | | | | | Jun-binding |
| | | NO: 121) | | | | | protein mRN, |
| | | | | | | | 3′ end |
| L9.2.2.174 | 20 | TCCGCGGCCGCCGC | ggg | normal | mRNA | NM_204217 | Gallus gallus |
| | | AGCCAT(SEQ ID | | | | | ribosomal |
| | | NO: 122) | | | | | protein S17- |
| | | | | | | | like |
| | | | | | | | (RPS17L), |
| | | | | | | | mRNA |
| L9.2.2.175 | 19 | CAGGGGAGGCAGAT | mismatch | normal | mRNA | XM_004950105 | PREDICTED: |
| | | CCAAA(SEQ ID | | | | | Gallus |
| | | NO: 123) | | | | | gallus |
| | | | | | | | cob(I)yrinic |
| | | | | | | | acid a,c- |
| | | | | | | | diamide |
| | | | | | | | adenosyltransferase, |
| | | | | | | | mitochondrial- |
| | | | | | | | like |
| | | | | | | | (LOC100859013), |
| | | | | | | | transcript |
| | | | | | | | variant X10, |
| | | | | | | | mRNA |
| L9.2.2.176 | 20 | TGGCACGGGGAAAG | ggg | normal | mRNA | NM_001006190 | Gallus gallus |
| | | CACGAC(SEQ ID | | | | | protein |
| | | NO: 124) | | | | | phosphatase |
| | | | | | | | 1, catalytic |
| | | | | | | | subunit, |
| | | | | | | | gamma |
| | | | | | | | isozyme |
| | | | | | | | (PPP1CC), |
| | | | | | | | mRNA |
| L9.2.2.177 | 20 | TTGAAGGCCGAAGT | ggg | normal | rRNA | JN639848 | Gallus gallus |
| | | GGAGCA(SEQ ID | | | | | 28S |
| | | NO: 125) | | | | | ribosomal |
| | | | | | | | RNA, partial |
| | | | | | | | sequence |
| L9.2.2.178 | 20 | CAAACGTTTGAAGA | tgg | normal | mRNA | NM_001006345 | Gallus gallus |
| | | GGCTGT(SEQ ID | | | | | ribosomal |
| | | NO: 126) | | | | | protein L7 |
| | | | | | | | (RPL7), |
| | | | | | | | mRNA |
| L9.2.2.179 | 20 | TGCGGAGCACCGCTCGTGGT | | | | | |
| | | (SEQ ID NO: 127) | | | | | |
| L9.2.2.180 | 18 | GTGCCCATCCCGCC | ccgg (at | normal | mRNA | XM_422813 | PREDICTED: |
| | | CAAC(SEQ ID | +1) | | | | Gallus |
| | | NO: 128) | | | | | gallus NMD3 |
| | | | | | | | homolog (S. cerevisiae) |
| | | | | | | | (NMD3), |
| | | | | | | | mRNA |
| L9.2.2.181 | 20 | CGGCCCTGCGTCAG | cgg | normal | mRNA | XM_424392 | PREDICTED: |
| | | GTACAC(SEQ ID | | | | | Gallus |
| | | NO: 129) | | | | | gallus TM2 |
| | | | | | | | domain |
| | | | | | | | containing 2 |
| | | | | | | | (TM2D2), |
| | | | | | | | mRNA |
| L9.2.2.182 | 20 | TCTGATGATGACAT | tgg | normal | mRNA | XM_424134 | PREDICTED: |
| | | GGGATT(SEQ ID | | | | | Gallus |
| | | NO: 130) | | | | | gallus |
| | | | | | | | ribosomal |
| | | | | | | | protein, large, |
| | | | | | | | P2 (RPLP2), |
| | | | | | | | mRNA |
| L9.2.2.183 | 20 | GGGCTCTGAGCAGC | tgg | normal | mRNA | NM_001031458 | Gallus gallus |
| | | CTGAGC(SEQ ID | | | | | nudix |
| | | NO: 131) | | | | | (nucleoside |
| | | | | | | | diphosphate |
| | | | | | | | linked moiety |
| | | | | | | | X)-type motif |
| | | | | | | | 19 |
| | | | | | | | (NUDT19), |
| | | | | | | | mRNA |
| L9.2.2.184 | 20 | CATCGAGCTGGTCA | agg | normal | mRNA | NM_001276303 | Gallus gallus |
| | | TGTCCC(SEQ ID | | | | | nascent |
| | | NO: 132) | | | | | polypeptide- |
| | | | | | | | associated |
| | | | | | | | complex |
| | | | | | | | alpha subunit |
| | | | | | | | (NACA), |
| | | | | | | | mRNA |
| L9.2.2.185 | 20 | AATGGTGCAACCGC | ggg | normal | mRNA | KP742951 | Gallus gallus |
| | | TATTAA(SEQ ID | | | | | breed Rugao |
| | | NO: 133) | | | | | yellow |
| | | | | | | | chicken |
| | | | | | | | mitochondrion, |
| | | | | | | | complete |
| | | | | | | | genome |
| L9.2.2.186 | 20 | TCCGTGCTGCTGGG | ggg | normal | mRNA | XM_003642618 | PREDICTED: |
| | | CGGCGA(SEQ ID | | | | | Gallus |
| | | NO: 134) | | | | | gallus |
| | | | | | | | ragulator |
| | | | | | | | complex |
| | | | | | | | protein |
| | | | | | | | LAMTOR2- |
| | | | | | | | like |
| | | | | | | | (LOC100859842), |
| | | | | | | | partial |
| | | | | | | | mRNA |
| L9.2.2.187 | 20 | GGCCGGGACTGCGCGCACAG | | | | | |
| | | (SEQ ID NO: 135) | | | | | |
| L9.2.2.188 | 20 | CTGGTGAAGTACAT | cgg | normal | mRNA | NM_205047 | Gallus gallus |
| | | GAACTC(SEQ ID | | | | | NME/NM23 |
| | | NO: 136) | | | | | nucleoside |
| | | | | | | | diphosphate |
| | | | | | | | kinase 2 |
| | | | | | | | (NME2), |
| | | | | | | | mRNA |
| L9.2.2.189 | 20 | TGACTAGTCCCACT | cgg | normal | mRNA | KP742951 | Gallus gallus |
| | | TATAAT(SEQ ID | | | | | breed Rugao |
| | | NO: 137) | | | | | yellow |
| | | | | | | | chicken |
| | | | | | | | mitochondrion, |
| | | | | | | | complete |
| | | | | | | | genome |
| L9.2.2.190 | 20 | CCGCCGCCTCCCGCCCCTAT | | | | | |
| | | (SEQ ID NO: 138) | | | | | |
| L9.2.2.191 | 20 | TCCCTAGCATTCGA | agg | normal | mRNA | AJ291765 | Gallus gallus |
| | | GACAAC(SEQ ID | | | | | mRNA for |
| | | NO: 139) | | | | | U2snRNP |
| | | | | | | | auxiliary |
| | | | | | | | factor small |
| | | | | | | | subunit class |
| | | | | | | | 3, (truncated), |
| | | | | | | | (U2AF1 |
| | | | | | | | gene) |
| L9.2.2.192 | 20 | CCACATGGAGCAGC | ggg | normal | mRNA | NM_001006318 | Gallus gallus |
| | | CAGCCT(SEQ ID | | | | | RNA binding |
| | | NO: 140) | | | | | motif protein |
| | | | | | | | 7 (RBM7), |
| | | | | | | | mRNA |
| L9.2.2.193 | 19 | TTCTAAAACCTTTG | agg | normal | mRNA | NM_001031506 | Gallus gallus |
| | | TGCAC(SEQ ID | | | | | solute carrier |
| | | NO: 141) | | | | | family 25 |
| | | | | | | | (mitochondrial |
| | | | | | | | folate |
| | | | | | | | carrier), |
| | | | | | | | member 32 |
| | | | | | | | (SLC25A32), |
| | | | | | | | mRNA |
| L9.2.2.194 | 20 | CCGCCACACACGCA | ggg | reverse | mRNA | NM_001030649 | Gallus gallus |
| | | GAGAAC(SEQ ID | | | | | eukaryotic |
| | | NO: 142) | | | | | translation |
| | | | | | | | initiation |
| | | | | | | | factor 4A3 |
| | | | | | | | (EIF4A3), |
| | | | | | | | mRNA |
| L9.2.2.195 | 19 | TTTAACGAGGATCC | agg | normal | rRNA | HQ873432 | Gallus gallus |
| | | ATTGG(SEQ ID | | | | | isolate ML48 |
| | | NO: 143) | | | | | 18S |
| | | | | | | | ribosomal |
| | | | | | | | RNA gene, |
| | | | | | | | partial |
| | | | | | | | sequence |
| L9.2.2.201 | 20 | CCTTCGGAGAGGTG | cgg | normal | mRNA | KJ617062 | Gallus gallus |
| | | TCCTCC(SEQ ID | | | | | gallus breed |
| | | NO: 144) | | | | | Sanhuang |
| | | | | | | | broiler akirin |
| | | | | | | | 2 mRNA, |
| | | | | | | | complete eds |
| L9.2.2.202 | 20 | CCCTCAGCGCGCCC | ggg | normal | mRNA | XM_004942331 | PREDICTED: |
| | | AACCGG(SEQ ID | | | | | Gallus |
| | | NO: 145) | | | | | gallus WD |
| | | | | | | | repeat domain |
| | | | | | | | 11 (WDR11), |
| | | | | | | | transcript |
| | | | | | | | variant X10, |
| | | | | | | | mRNA |
| L9.2.2.203 | 20 | CAGCCGCCATGCCT | cgg | normal | mRNA | NM_001252255 | Gallus gallus |
| | | GCCCTC(SEQ ID | | | | | ribosomal |
| | | NO: 146) | | | | | protein L32 |
| | | | | | | | (RPL32), |
| | | | | | | | mRNA |
| L9.2.2.204 | 20 | AGAATAGTTTTATA | tgg | normal | mRNA | NM_001030916 | Gallus gallus |
| | | AACCAT(SEQ ID | | | | | WD repeat |
| | | NO: 147) | | | | | domain 77 |
| | | | | | | | (WDR77), |
| | | | | | | | mRNA |
| L9.2.2.205 | 20 | TTTTGTTGGTTTTCG | ggg | reverse | mRNA | L48915 | Gallus gallus |
| | | GAAAC(SEQ ID | | | | | clone |
| | | NO: 148) | | | | | CDNA34A, |
| | | | | | | | mRNA |
| | | | | | | | sequence |
| L9.2.2.206 | 20 | ACCCTCCGCGGTAC | ggg | normal | mRNA | NM_001004378 | Gallus gallus |
| | | CCTGAA(SEQ ID | | | | | guanine |
| | | NO: 149) | | | | | nucleotide |
| | | | | | | | binding |
| | | | | | | | protein (G |
| | | | | | | | protein), beta |
| | | | | | | | Polypeptide |
| | | | | | | | 2-like 1 |
| | | | | | | | (GNB2L1), |
| | | | | | | | mRNA |
| L9.2.2.207 | 19 | TGAGAATGAGAAGA | ggg | normal | mRNA | XM_004944589 | PREDICTED: |
| | | ACAAT(SEQ ID | | | | | Gallus |
| | | NO: 150) | | | | | gallus |
| | | | | | | | ubiquinol- |
| | | | | | | | cytochrome c |
| | | | | | | | reductase |
| | | | | | | | core protein I |
| | | | | | | | (UQCRC1), |
| | | | | | | | transcript |
| | | | | | | | variant X3, |
| | | | | | | | mRNA |
| L9.2.2.208 | 20 | TGTAGACAAAAACT | agg | normal | mRNA | XM_004946901 | PREDICTED: |
| | | CAGCTC(SEQ ID | | | | | Gallus |
| | | NO: 151) | | | | | gallus RNA- |
| | | | | | | | binding |
| | | | | | | | protein 39- |
| | | | | | | | like |
| | | | | | | | (LOC100858247), |
| | | | | | | | transcript |
| | | | | | | | variant X12, |
| | | | | | | | mRNA |
| L9.2.2.209 | 21 | GGCCCGATCTGGAA | tgg | normal | mRNA | NM_001030619 | Gallus gallus |
| | | TGAAGAT(SEQ ID | | | | | ribosomal |
| | | NO: 152) | | | | | protein S14 |
| | | | | | | | (RPS14), |
| | | | | | | | mRNA |
| L9.2.2.210 | 20 | GCGAGCGGTGCGGAGACCAC | | | | | |
| | | (SEQ ID NO: 153) | | | | | |
| L9.2.2.211 | 20 | AAGGGCACAGTGCT | cgg | normal | mRNA | AY389963 | Gallus gallus |
| | | GCTGTC(SEQ ID | | | | | ribosomal |
| | | NO: 154) | | | | | protein L18 |
| | | | | | | | mRNA, |
| | | | | | | | partial eds |
| L9.2.2.212 | 20 | CGTGGTGGCCTACC | tgg | normal | mRNA | XM_003643500 | PREDICTED: |
| | | TGGTGC(SEQ ID | | | | | Gallus |
| | | NO: 155) | | | | | gallus |
| | | | | | | | RTN3w |
| | | | | | | | (RTN3), |
| | | | | | | | mRNA |
| L9.2.2.213 | 20 | CAGCCTTACAACAT | cgg | normal | mRNA | XM_003643075 | PREDICTED: |
| | | GTGATC(SEQ ID | | | | | Gallus |
| | | NO: 156) | | | | | gallus general |
| | | | | | | | transcription |
| | | | | | | | factor IIH, |
| | | | | | | | Polypeptide 2, |
| | | | | | | | 44 kDa |
| | | | | | | | (GTF2H2), |
| | | | | | | | transcript |
| | | | | | | | variant X1, |
| | | | | | | | mRNA |
| L9.2.2.214 | 21 | CATTTCCAGCCCCA | tgg | | | | chr9: 14805792-14805812 |
| | | TCTGCCC(SEQ ID | | | | | |
| | | NO: 157) | | | | | |
| L9.2.2.215 | 20 | ACGGGCCGGTGGTG | ggg | reverse | rRNA | X51919 | Gallus gallus |
| | | CGCCCG(SEQ ID | | | | | large-subunit |
| | | NO: 158) | | | | | ribosomal |
| | | | | | | | RNA D3 |
| | | | | | | | domain |
| L9.2.2.216 | 20 | TCCAAGGCGGGGTT | cagg (at | reverse | mRNA | NM_204987 | Gallus gallus |
| | | GTTCTC(SEQ ID | +1) | | | | ribosomal |
| | | NO: 159) | | | | | protein, large, |
| | | | | | | | P0 (RPLP0), |
| | | | | | | | mRNA |
| L9.2.2.217 | 20 | CGGCCTCAACAAGG | cgg | normal | mRNA | NM_001031556 | Gallus gallus |
| | | CTGAGA(SEQ ID | | | | | phosphoglycerate |
| | | NO: 160) | | | | | mutase 1 |
| | | | | | | | (brain) |
| | | | | | | | (PGAM1), |
| | | | | | | | mRNA |
| L9.2.2.218 | 20 | ACGGGCTGCTGCTGTGAGCA | | | | | |
| | | (SEQ ID NO: 161) | | | | | |
| L9.2.2.219 | 20 | CGCCTCTCCCCCGC | cgg | normal | mRNA | NM_001287205 | Gallus gallus |
| | | GGGTGC(SEQ ID | | | | | ribosomal |
| | | NO: 162) | | | | | protein S27a |
| | | | | | | | (RPS27A), |
| | | | | | | | mRNA |
| L9.2.2.220 | 20 | TAGCTACCCGGCGT | tgg | normal | mRNA | KP742951 | Gallus gallus |
| | | AAAGAG(SEQ ID | | | | | breed Rugao |
| | | NO: 163) | | | | | yellow |
| | | | | | | | chicken |
| | | | | | | | mitochondrion, |
| | | | | | | | complete |
| | | | | | | | genome |
| L9.2.2.221 | 20 | GGGACCGCCGTTCTACGTTC | | | | | |
| | | (SEQ ID NO: 164) | | | | | |
| L9.2.2.222 | 20 | CCATGATTAAGAGG | cgg | normal | rRNA | HQ873432 | Gallus gallus |
| | | GACGGC(SEQ ID | | | | | isolate ML48 |
| | | NO: 165) | | | | | 18S |
| | | | | | | | ribosomal |
| | | | | | | | RNA gene, |
| | | | | | | | partial |
| | | | | | | | sequence |
| L9.2.2.223 | 20 | CGGCACGATGTTTT | tgg | normal | mRNA | XM_004938806 | PREDICTED: |
| | | TAACGC(SEQ ID | | | | | Gallus |
| | | NO: 166) | | | | | gallus |
| | | | | | | | mitochondrial |
| | | | | | | | ribosomal |
| | | | | | | | protein 63 |
| | | | | | | | (MRP63), |
| | | | | | | | transcript |
| | | | | | | | variant X2, |
| | | | | | | | mRNA |
| L9.2.2.224 | 20 | CTGAGGAGCAGGCT | tgg | normal | mRNA | XM_004942078 | PREDICTED: |
| | | AACAAT(SEQ ID | | | | | Gallus |
| | | NO: 167) | | | | | gallus |
| | | | | | | | neurotrypsin- |
| | | | | | | | like |
| | | | | | | | (LOC423740), |
| | | | | | | | transcript |
| | | | | | | | variant X2, |
| | | | | | | | mRNA |
| L9.2.2.225 | 20 | CCGCCGCCAAGGGTAAGAAG | | | | | |
| | | (SEQ ID NO: 168) | | | | | |
| L9.2.2.226 | 20 | CACCTTGCCCAGAT | ggg | reverse | mRNA | NM_001199857 | Gallus gallus |
| | | CCTGCC(SEQ ID | | | | | cyclin- |
| | | NO: 169) | | | | | dependent |
| | | | | | | | kinase 2 |
| | | | | | | | (CDK2), |
| | | | | | | | mRNA |
| L9.2.2.227 | 20 | CGGGGGCACGGAGC | ggg | normal | mRNA | XM_004950206 | PREDICTED: |
| | | ACACAT(SEQ ID | | | | | Gallus |
| | | NO: 170) | | | | | gallus nuclear |
| | | | | | | | calmodulin- |
| | | | | | | | binding |
| | | | | | | | protein |
| | | | | | | | (URP), |
| | | | | | | | mRNA |
| L9.2.2.228 | 20 | AACATCTCTCCCTT | tgg | normal | mRNA | NM_204987 | Gallus gallus |
| | | CTCCTT(SEQ ID | | | | | ribosomal |
| | | NO: 171) | | | | | protein, large, |
| | | | | | | | P0 (RPLP0), |
| | | | | | | | mRNA |
| L9.2.2.229 | 20 | CGTCCCGGTTCGGC | cgg | normal | mRNA | KP064313 | Gallus gallus |
| | | CCGGTC(SEQ ID | | | | | GABA(A) |
| | | NO: 172) | | | | | reeeptor- |
| | | | | | | | associated |
| | | | | | | | protein |
| | | | | | | | mRNA, |
| | | | | | | | complete cds |
| L9.2.2.230 | 20 | CTGGTGAAGTACAT | cgg | normal | mRNA | NM_205047 | Gallus gallus |
| | | GAACTC(SEQ ID | | | | | NME/NM23 |
| | | NO: 173) | | | | | nucleoside |
| | | | | | | | diphosphate |
| | | | | | | | kinase 2 |
| | | | | | | | (NME2), |
| | | | | | | | mRNA |
| L9.2.2.231 | 20 | GCGCGGCCGTGCTG | agg | normal | mRNA | NM_001030989 | Gallus gallus |
| | | CCGAGG(SEQ ID | | | | | SH3-domain |
| | | NO: 174) | | | | | binding |
| | | | | | | | protein 5 |
| | | | | | | | (BTK- |
| | | | | | | | associated) |
| | | | | | | | (SH3BP5), |
| | | | | | | | mRNA |
| L9.2.2.232 | 20 | CCCAACCCGGGCAT | cgg | normal | mRNA | NM_204780 | Gallus gallus |
| | | GCTGTT(SEQ ID | | | | | nudix |
| | | NO: 175) | | | | | (nucleoside |
| | | | | | | | diphosphate |
| | | | | | | | linked moiety |
| | | | | | | | X)-type motif |
| | | | | | | | 16-like 1 |
| | | | | | | | (NUDT16L1), |
| | | | | | | | mRNA |
| L9.2.2.233 | 20 | CGTCGCCAAGACCT | cgg | normal | mRNA | CR387434 | Gallus gallus |
| | | CGAGGA(SEQ ID | | | | | finished |
| | | NO: 176) | | | | | cDNA, clone |
| | | | | | | | ChEST26e5 |
| L9.2.2.234 | 19 | CTTTCAATGGGTAA | ccgg (at | normal | rRNA | FM165415 | Gallus gallus |
| | | GACGC(SEQ ID | +1) | | | | 28S rRNA |
| | | NO: 177) | | | | | gene, clone |
| | | | | | | | GgLSU-1 |
| L9.2.2.235 | 20 | AAGTAGTGCTGCGACCAGAC | | | | | |
| | | (SEQ ID NO: 178) | | | | | |
| L9.2.2.236 | 20 | GGGTTCTGCTCTGCGGCTTC | | | | | |
| | | (SEQ ID NO: 179) | | | | | |
| L9.2.2.237 | 20 | GGCTCCCCTCTGTGCCCCGC | | | | | |
| | | (SEQ ID NO: 180) | | | | | |
| L9.2.2.238 | 20 | CGGCTCCGGGGCCG | ggg | normal | mRNA | NM_001302195 | Gallus gallus |
| | | GCGGGG(SEQ ID | | | | | translocase of |
| | | NO: 181) | | | | | inner |
| | | | | | | | mitochondrial |
| | | | | | | | membrane 13 |
| | | | | | | | homolog |
| | | | | | | | (yeast) |
| | | | | | | | (TIMM13), |
| | | | | | | | mRNA |
| L9.2.2.239 | 20 | CATGGCGGGAACCGCGGCGA | | | | | |
| | | (SEQ ID NO: 182) | | | | | |
| L9.2.2.240 | 20 | GAGTCCATTTTGGGGGGCGG | | | | | |
| | | (SEQ ID NO: 183) | | | | | |
| L9.2.2.241 | 20 | CGCTCCGGGGACAG | gtgg (at | normal | mRNA | AB556518 | Gallus gallus |
| | | CGTCAG(SEQ ID | +1) | | | | DNA, CENP- |
| | | NO: 184) | | | | | A associated |
| | | | | | | | sequence, |
| | | | | | | | partial |
| | | | | | | | sequence, |
| | | | | | | | clone: |
| | | | | | | | CAIP#220 |
| L9.2.2.242 | 20 | TATTCAAACGAGAG | agg | normal | rRNA | JN639848 | Gallus gallus |
| | | CTTTGA(SEQ ID | | | | | 28S |
| | | NO: 185) | | | | | ribosomal |
| | | | | | | | RNA, partial |
| | | | | | | | sequence |
| L9.2.2.243 | 19 | ACCGGAGCTCTTCT | cgg | normal | mRNA | NM_001006308 | Gallus gallus |
| | | GCAAT(SEQ ID | | | | | small nuclear |
| | | NO: 186) | | | | | ribonucleoprotein |
| | | | | | | | 40 kDa |
| | | | | | | | (U5) |
| | | | | | | | (SNRNP40), |
| | | | | | | | mRNA |
| L9.2.2.244 | 20 | CACGGCCTCATCCG | cgg | normal | mRNA | NM_001277880 | Gallus gallus |
| | | TAAGTA(SEQ ID | | | | | ribosomal |
| | | NO: 187) | | | | | protein S29 |
| | | | | | | | (RPS29), |
| | | | | | | | mRNA |
| L9.2.2.245 | 20 | CCTCACCTTCATTG | cgg | reverse | mRNA | NM_001004410 | Gallus gallus |
| | | CGCCGC(SEQ ID | | | | | phosphatidylinositol- |
| | | NO: 188) | | | | | 4,5- |
| | | | | | | | bisphosphate |
| | | | | | | | 3-kinase, |
| | | | | | | | catalytic |
| | | | | | | | subunit alpha |
| | | | | | | | (PIK3CA), |
| | | | | | | | mRNA |
| L9.2.2.246 | 20 | GAGGAAGCAGAGCG | gcgg (at | normal | mRNA | XM_003641094 | PREDICTED: |
| | | GCTATG(SEQ ID | +1) | | | | Gallus |
| | | NO: 189) | | | | | gallus |
| | | | | | | | ribosomal |
| | | | | | | | protein L36a |
| | | | | | | | (RPL36A), |
| | | | | | | | transcript |
| | | | | | | | variant X1, |
| | | | | | | | mRNA |
| L9.2.2.247 | 20 | TGTCATAGGTTAAC | tgg | normal | mRNA | KP742951 | Gallus gallus |
| | | CTGCTT(SEQ ID | | | | | breed Rugao |
| | | NO: 190) | | | | | yellow |
| | | | | | | | chicken |
| | | | | | | | mitochondrion, |
| | | | | | | | complete |
| | | | | | | | genome |
| L9.2.2.248 | 20 | AAGTAGTGCTGCGACCAGAC | | | | | |
| | | (SEQ ID NO: 191) | | | | | |
| L9.2.2.249 | 20 | CCCGCCCCGCCGCG | agg | normal | mRNA | CR387434 | Gallus gallus |
| | | CATTCC(SEQ ID | | | | | finished |
| | | NO: 192) | | | | | cDNA, clone |
| | | | | | | | ChEST26e5 |
| L9.2.2.250 | 20 | AATGAAGCGCGGGT | cgg | | | | chrUn_AADN03019346: |
| | | AAACGG(SEQ ID | | | | | 869-888 |
| | | NO: 193) | | | | | |
| L9.2.2.251 | 20 | CAACCTCTTGTGTA | tgg | normal | mRNA | NM_204852 | Gallus gallus |
| | | CAGAGC(SEQ ID | | | | | retinoblastom |
| | | NO: 194) | | | | | a binding |
| | | | | | | | protein 4 |
| | | | | | | | (RBBP4), |
| | | | | | | | mRNA |
| L9.2.2.252 | 20 | TGCCAGGAGGGCTC | ggg | | | | chr19: 8445596-8445615 |
| | | TGGAAT(SEQ ID | | | | | |
| | | NO: 195) | | | | | |
| L9.2.2.253 | 20 | GAAGTGGCGCAGCG | ggg | normal | mRNA | NM_001006218 | Gallus gallus |
| | | CGCGGC(SEQ ID | | | | | coiled-coil- |
| | | NO: 196) | | | | | helix-coiled- |
| | | | | | | | coil-helix |
| | | | | | | | domain |
| | | | | | | | containing 2 |
| | | | | | | | (CHCHD2), |
| | | | | | | | mRNA |
| L9.2.2.254 | 20 | GCTCCCCTCTGTGA | agg | normal | mRNA | KC610517 | Gallus gallus |
| | | ATAACC(SEQ ID | | | | | endogenous |
| | | NO: 197) | | | | | virus ALVE- |
| | | | | | | | B11 genomic |
| | | | | | | | sequence |
| L9.2.2.255 | 20 | TTCGTCGCTACAGG | cgg | normal | mRNA | KP742951 | Gallus gallus |
| | | GTTCCA(SEQ ID | | | | | breed Rugao |
| | | NO: 198) | | | | | yellow |
| | | | | | | | chicken |
| | | | | | | | mitochondrion, |
| | | | | | | | complete |
| | | | | | | | genome |
| L9.2.2.256 | 20 | GAGAAGTGCATGGA | cgg | normal | mRNA | NM_001302110 | Gallus gallus |
| | | CAAGCC(SEQ ID | | | | | translocase of |
| | | NO: 199) | | | | | inner |
| | | | | | | | mitochondrial |
| | | | | | | | membrane 8 |
| | | | | | | | homolog A |
| | | | | | | | (yeast) |
| | | | | | | | (TIMM8A), |
| | | | | | | | mRNA |
| L9.2.2.257 | 19 | TCCCCCACAATTAT | ccgg (at | normal | mRNA | KP742951 | Gallus gallus |
| | | CTTAA(SEQ ID | +1) | | | | breed Rugao |
| | | NO: 200) | | | | | yellow |
| | | | | | | | chicken |
| | | | | | | | mitochondrion, |
| | | | | | | | complete |
| | | | | | | | genome |
| L9.2.2.258 | 20 | GGCCGCCTGGCACA | ggg | normal | mRNA | BX931917 | Gallus gallus |
| | | CGAGGT(SEQ ID | | | | | finished |
| | | NO: 201) | | | | | cDNA, clone |
| | | | | | | | ChEST790c21 |
| L9.2.2.259 | 20 | CACACCCCAACTGT | ggg | normal | mRNA | KP742951 | Gallus gallus |
| | | CCAAAA(SEQ ID | | | | | breed Rugao |
| | | NO: 202) | | | | | yellow |
| | | | | | | | chicken |
| | | | | | | | mitochondrion, |
| | | | | | | | complete |
| | | | | | | | genome |
| L9.2.2.260 | 20 | TGTGATGCCCTTAG | ggg | normal | rRNA | FM165414 | Gallus gallus |
| | | ATGTCC(SEQ ID | | | | | 18S rRNA |
| | | NO: 203) | | | | | gene, clone |
| | | | | | | | GgSSU-1 |
| L9.2.2.261 | 20 | CCGTGCGGGGCGGG | cgg | | | | chr8: 13622296-13622315 |
| | | CAGGTA(SEQ ID | | | | | |
| | | NO: 204) | | | | | |
| L9.2.2.262 | 20 | CGCGGCCACGTCCAGCCCCA | | | | | |
| | | (SEQ ID NO: 205) | | | | | |
| L9.2.2.263 | 19 | TTTAACGAGGATCC | agg | normal | rRNA | HQ873432 | Gallus gallus |
| | | ATTGG(SEQ ID | | | | | isolate ML48 |
| | | NO: 206) | | | | | 18S |
| | | | | | | | ribosomal |
| | | | | | | | RNA gene, |
| | | | | | | | partial |
| | | | | | | | sequence |
| L9.2.2.264 | 20 | GCGGCCCCCGGCCC | agg | normal | mRNA | NM_204853 | Gallus gallus |
| | | GGATGA(SEQ ID | | | | | xeroderma |
| | | NO: 207) | | | | | pigmentosum, |
| | | | | | | | complementation |
| | | | | | | | group A |
| | | | | | | | (XPA), |
| | | | | | | | mRNA |
| L9.2.2.265 | 20 | AAGTTCAGCAAATC | tgg | normal | mRNA | FJ881855 | Gallus gallus |
| | | CGCTAC(SEQ ID | | | | | eukaryotic |
| | | NO: 208) | | | | | translation |
| | | | | | | | elongation |
| | | | | | | | factor 2 |
| | | | | | | | (EEF2) gene, |
| | | | | | | | exon 6 and |
| | | | | | | | partial eds |
| L9.2.2.266 | 20 | TGTGCGGTCCGACT | agg | normal | mRNA | XM_004939436 | PREDICTED: |
| | | GCTGTG(SEQ ID | | | | | Gallus |
| | | NO: 209) | | | | | gallus |
| | | | | | | | methyltransferase |
| | | | | | | | like 6 |
| | | | | | | | (METTL6), |
| | | | | | | | transcript |
| | | | | | | | variant X5, |
| | | | | | | | mRNA |
| L9.2.2.267 | 20 | TCGCCGGCGGTGCG | cgg | normal | rRNA | FM165415 | Gallus gallus |
| | | GAGCCG(SEQ ID | | | | | 28S rRNA |
| | | NO: 210) | | | | | gene, clone |
| | | | | | | | GgLSU-1 |
| L9.2.2.268 | 20 | TCGTCCACCTTTGC | ccgg (at | reverse | mRNA | L13234 | Gallus gallus |
| | | TTTCTT(SEQ ID | +1 | | | | Jun-binding |
| | | NO: 211) | | | | | protein mRN, |
| | | | | | | | 3′ end |
| L9.2.2.269 | 20 | TCGCCCGCTGCTTT | cgg | normal | mRNA | BX932373 | Gallus gallus |
| | | AAGAAC(SEQ ID | | | | | finished |
| | | NO: 212) | | | | | cDNA, clone |
| | | | | | | | ChEST98d21 |
| L9.2.2.270 | 20 | ACAAAATGCTGTCC | ggg | normal | mRNA | L13234 | Gallus gallus |
| | | TGCGCC(SEQ ID | | | | | Jun-binding |
| | | NO: 213) | | | | | protein mRN, |
| | | | | | | | 3′ end |
| L9.2.2.271 | 21 | TGTTGCTGTTACTA | tgg | normal | mRNA | NM_001277729 | Gallus gallus |
| | | TTTTCTT(SEQ ID | | | | | isoamyl |
| | | NO: 214) | | | | | acetate- |
| | | | | | | | hydrolyzing |
| | | | | | | | esterase 1 |
| | | | | | | | homolog (S. cerevisiae) |
| | | | | | | | (IAH1), |
| | | | | | | | mRNA |
| L9.2.2.272 | 20 | GATGGAGTCGTACT | agg | normal | mRNA | XM_420600 | PREDICTED: |
| | | ACTCAG(SEQ ID | | | | | Gallus |
| | | NO: 215) | | | | | gallus G-rich |
| | | | | | | | RNA |
| | | | | | | | sequence |
| | | | | | | | binding factor |
| | | | | | | | 1 (GRSF1), |
| | | | | | | | transcript |
| | | | | | | | variant X2, |
| | | | | | | | mRNA |
| L9.2.2.273 | 20 | GACCGCCTGGCTGCGTTCTA | | | | | |
| | | (SEQ ID NO: 216) | | | | | |
| L9.2.2.274 | 20 | TCCCTGCCCTTTGT | mismatch | normal | rRNA | HQ873432 | Gallus gallus |
| | | ACACAC(SEQ ID | | | | | isolate ML48 |
| | | NO: 217) | | | | | 18S |
| | | | | | | | ribosomal |
| | | | | | | | RNA gene, |
| | | | | | | | partial |
| | | | | | | | sequence |
| L9.2.2.275 | 20 | CGGAAAGACGAAGGTCCCGA | | | | | |
| | | (SEQ ID NO: 218) | | | | | |
| L9.2.2.276 | 19 | CCTGTGCTAATCCT | cgg | normal | mRNA | NM_204985 | Gallus gallus |
| | | GCAAA(SEQ ID | | | | | phosphoglyce |
| | | NO: 219) | | | | | rate kinase 1 |
| | | | | | | | (PGK1), |
| | | | | | | | mRNA |
| L9.2.2.277 | 20 | AAACAACCAGCCTA | cgg | normal | mRNA | KP742951 | Gallus gallus |
| | | CTTATT(SEQ ID | | | | | breed Rugao |
| | | NO: 220) | | | | | yellow |
| | | | | | | | chicken |
| | | | | | | | mitochondrion, |
| | | | | | | | complete |
| | | | | | | | genome |
| L9.2.2.278 | 20 | ATGAACAGCGCCAG | ggg | reverse | mRNA | CR387434 | Gallus gallus |
| | | CAGCCA(SEQ ID | | | | | finished |
| | | NO: 221) | | | | | cDNA, clone |
| | | | | | | | ChEST26e5 |
| L9.2.2.279 | 20 | TCCCAGCCAGTGAA | cgg | normal | mRNA | XM_004941162 | PREDICTED: |
| | | CACCTC(SEQ ID | | | | | Gallus |
| | | NO: 222) | | | | | gallus cyclin I |
| | | | | | | | (CCNI), |
| | | | | | | | transcript |
| | | | | | | | variant X3, |
| | | | | | | | mRNA |
| L9.2.2.280 | 20 | CGTCGCAGAGCATCGCCCAG | | | | | |
| | | (SEQ ID NO: 223) | | | | | |
| L9.2.2.281 | 20 | CGCGGCCTCGGGCC | cgg | | | | chr9: 23080146-23080165 |
| | | CGAACC(SEQ ID | | | | | |
| | | NO: 224) | | | | | |
| L9.2.2.282 | 20 | GAAGTCGCGCCCAGTAATGC | | | | | |
| | | (SEQ ID NO: 225) | | | | | |
| L9.2.2.283 | 20 | GAAGGCCCCGGGCG | cgg | normal | mRNA | X51919 | Gallus gallus |
| | | CACCAC(SEQ ID | | | | | large-subunit |
| | | NO: 226) | | | | | ribosomal |
| | | | | | | | RNA D3 |
| | | | | | | | domain |
| L9.2.2.284 | 20 | CACACCTGCCTTGC | acgg (at | reverse | mRNA | NM_001006138 | Gallus gallus |
| | | CTCTTG(SEQ ID | +1) | | | | RuvB-like 1 |
| | | NO: 227) | | | | | (E. coli) |
| | | | | | | | (RUVBL1), |
| | | | | | | | mRNA |
| L9.2.2.285 | 20 | TTCCTAGCACCAGT | cgg | normal | mRNA | NM_001031513 | Gallus gallus |
| | | TTTTAG(SEQ ID | | | | | STT3B, |
| | | NO: 228) | | | | | subunit of the |
| | | | | | | | oligosaccharyltransferase |
| | | | | | | | complex |
| | | | | | | | (catalytic) |
| | | | | | | | (STT3B), |
| | | | | | | | mRNA |
| L9.2.2.286 | 20 | AGCATACCAATCAG | cgg | normal | mRNA | KP742951 | Gallus gallus |
| | | CTACGC(SEQ ID | | | | | breed Rugao |
| | | NO: 229) | | | | | yellow |
| | | | | | | | chicken |
| | | | | | | | mitochondrion, |
| | | | | | | | complete |
| | | | | | | | genome |
| L9.2.2.287 | 20 | TTTGGCAGCCCGTG | tgg | normal | mRNA | NM_001007823 | Gallus gallus |
| | | CTATTG(SEQ ID | | | | | ribosomal |
| | | NO: 230) | | | | | protein SA |
| | | | | | | | (RPSA), |
| | | | | | | | mRNA |
| L9.2.2.288 | 20 | GCTCCATTGGAGGGCAAGTC | | | | | |
| | | (SEQ ID NO: 231) | | | | | |
| L9.2.2.289 | 20 | TGGAGTGGGCTTCA | ggg | normal | mRNA | NM_001277755 | Gallus gallus |
| | | AGAAGC(SEQ ID | | | | | ribosomal |
| | | NO: 232) | | | | | protein L31 |
| | | | | | | | (RPL31), |
| | | | | | | | mRNA |
| L9.2.2.290 | 20 | GGGGTCCTTGGGGGTCTCAG | | | | | |
| | | (SEQ ID NO: 233) | | | | | |
| L9.2.2.291 | 20 | CACTGATTTCCCCT | agg | normal | mRNA | KP742951 | Gallus gallus |
| | | CTTCAC(SEQ ID | | | | | breed Rugao |
| | | NO: 234) | | | | | yellow |
| | | | | | | | chicken |
| | | | | | | | mitochondrion, |
| | | | | | | | complete |
| | | | | | | | genome |
| L9.2.2.292 | 20 | TTCATCCTCACTGCCCCCCC | | | | | |
| | | (SEQ ID NO: 235) | | | | | |
| L9.2.2.293 | 20 | ACTTTACTTGTGGT | agg | normal | mRNA | XM_004943373 | PREDICTED: |
| | | GTGACC(SEQ ID | | | | | Gallus |
| | | NO: 236) | | | | | gallus |
| | | | | | | | prothymosin, |
| | | | | | | | alpha |
| | | | | | | | (PTMA), |
| | | | | | | | transcript |
| | | | | | | | variant X4, |
| | | | | | | | mRNA |
| L9.2.2.294 | 19 | TTGTACTTCATTGC | cagg (at | normal | mRNA | NM_001031125 | Gallus gallus |
| | | TCCGA(SEQ ID | +1) | | | | septin 6 |
| | | NO: 237) | | | | | (SEPT6), |
| | | | | | | | mRNA |
| L9.2.2.295 | 20 | TATTAAATTAAAGCTCGTCC | | | | | |
| | | (SEQ ID NO: 238) | | | | | |
| L9.2.2.301 | 20 | AAGTGCTGTGCCGG | mismatch | normal | mRNA | KP742951 | Gallus gallus |
| | | CTATGC(SEQ ID | | | | | breed Rugao |
| | | NO: 239) | | | | | yellow |
| | | | | | | | chicken |
| | | | | | | | mitochondrion, |
| | | | | | | | complete |
| | | | | | | | genome |
| L9.2.2.302 | 20 | CATGATTAAGAGGG | ggg | normal | rRNA | HQ873432 | Gallus gallus |
| | | ACGGCC(SEQ ID | | | | | isolate ML48 |
| | | NO: 240) | | | | | 18S |
| | | | | | | | ribosomal |
| | | | | | | | RNA gene, |
| | | | | | | | partial |
| | | | | | | | sequence |
| L9.2.2.303 | 20 | GAGGGGCAACTGAGGGGCAG | | | | | |
| | | (SEQ ID NO: 241) | | | | | |
| L9.2.2.304 | 20 | AGTTACGGATCCGGCTTGCC | | | | | |
| | | (SEQ ID NO: 242) | | | | | |
| L9.2.2.305 | 20 | TCCATCCACGTGGG | ggg | normal | mRNA | BX934736 | Gallus gallus |
| | | CCAAGC(SEQ ID | | | | | finished |
| | | NO: 243) | | | | | cDNA, clone |
| | | | | | | | ChEST559b14 |
| L9.2.2.306 | 20 | TGTTGATCAGCAAA | ggg | normal | mRNA | NM_001097531 | Gallus gallus |
| | | AATGAA(SEQ ID | | | | | zinc finger |
| | | NO: 244) | | | | | protein 706 |
| | | | | | | | (ZNF706), |
| | | | | | | | mRNA |
| L9.2.2.307 | 20 | CTCAACAACTCTGA | ggg | normal | mRNA | XM_423974 | PREDICTED: |
| | | CCTGAT(SEQ ID | | | | | Gallus |
| | | NO: 245) | | | | | gallus RNA |
| | | | | | | | binding motif |
| | | | | | | | protein 34 |
| | | | | | | | (RBM34), |
| | | | | | | | mRNA |
| L9.2.2.308 | 20 | ATCACCCCTCCCCG | ggg | normal | mRNA | KP742951 | Gallus gallus |
| | | CACTGT(SEQ ID | | | | | breed Rugao |
| | | NO: 246) | | | | | yellow |
| | | | | | | | chicken |
| | | | | | | | mitochondrion, |
| | | | | | | | complete |
| | | | | | | | genome |
| L9.2.2.309 | 20 | GGGGAATGCGAGCGCTCAGT | | | | | |
| | | (SEQ ID NO: 247) | | | | | |
| L9.2.2.310 | 20 | CGGCACAATACGAA | cgg | reverse | rRNA | HQ873432 | Gallus gallus |
| | | TGCCCC(SEQ ID | | | | | isolate ML48 |
| | | NO: 248) | | | | | 18S |
| | | | | | | | ribosomal |
| | | | | | | | RNA gene, |
| | | | | | | | partial |
| | | | | | | | sequence |
| L9.2.2.311 | 20 | TATGGGCATCGGGA | agg | normal | rRNA | AY393838 | Gallus gallus |
| | | AGAGAA(SEQ ID | | | | | ribosomal |
| | | NO: 249) | | | | | protein L19 |
| | | | | | | | mRNA, |
| | | | | | | | partial cds |
| L9.2.2.312 | 20 | CACCTCGTCCTGCT | cgg | normal | mRNA | XM_424387 | PREDICTED: |
| | | ACGGGA(SEQ ID | | | | | Gallus |
| | | NO: 250) | | | | | gallus LSM1 |
| | | | | | | | homolog, U6 |
| | | | | | | | small nuclear |
| | | | | | | | RNA |
| | | | | | | | associated (S. cerevisiae) |
| | | | | | | | (LSM1), |
| | | | | | | | mRNA |
| L9.2.2.313 | 20 | CAGGGGGACTTCTA | tgg | normal | mRNA | NM_205086 | Gallus gallus |
| | | CTTCAC(SEQ ID | | | | | ferritin, heavy |
| | | NO: 251) | | | | | Polypeptide 1 |
| | | | | | | | (FTH1), |
| | | | | | | | mRNA |
| L9.2.2.314 | 20 | TGCGGGCACTACGG | ggg | normal | mRNA | NM_205390 | Gallus gallus |
| | | CTGAGA(SEQ ID | | | | | calcium- |
| | | NO: 252) | | | | | binding |
| | | | | | | | protein (P22), |
| | | | | | | | mRNA |
| L9.2.2.315 | 20 | GGGGAGGGCGGGAGCGATAG | | | | | |
| | | (SEQ ID NO: 253) | | | | | |
| L9.2.2.316 | 20 | CACGGCCTCATCCG | cgg | normal | mRNA | NM_001277880 | Gallus gallus |
| | | TAAGTA(SEQ ID | | | | | ribosomal |
| | | NO: 254) | | | | | protein S29 |
| | | | | | | | (RPS29), |
| | | | | | | | mRNA |
| L9.2.2.317 | 20 | ACCCGAGATTGAGC | agg | normal | rRNA | HQ873432 | Gallus gallus |
| | | AATAAC(SEQ ID | | | | | isolate ML48 |
| | | NO: 255) | | | | | 18S |
| | | | | | | | ribosomal |
| | | | | | | | RNA gene, |
| | | | | | | | partial |
| | | | | | | | sequence |
| L9.2.2.318 | 20 | CCTCTTCGGTACCT | cgg | reverse | mRNA | BX934562 | Gallus gallus |
| | | CCTCAG(SEQ ID | | | | | finished |
| | | NO: 256) | | | | | cDNA, clone |
| | | | | | | | ChEST28c10 |
| L9.2.2.319 | 20 | TCCCCTCGGGTCCATTATCG | | | | | |
| | | (SEQ ID NO: 257) | | | | | |
| L9.2.2.320 | 20 | AGCTGTACTTGTGG | agg | reverse | mRNA | NM_001030560 | Gallus gallus |
| | | CTGAGC(SEQ ID | | | | | glucose- |
| | | NO: 258) | | | | | fructose |
| | | | | | | | oxidoreduetase |
| | | | | | | | domain |
| | | | | | | | containing 2 |
| | | | | | | | (GFOD2), |
| | | | | | | | mRNA |
| L9.2.2.321 | 20 | TTCGGGGTTCTCCG | ggg | reverse | mRNA | X01613 | Gallus gallus |
| (Cμ | | CCATGG(SEQ ID | | | | | mRNA for |
| guide | | NO: 259) | | | | | mu |
| 3) | | | | | | | immunoglobulin |
| | | | | | | | heavy |
| | | | | | | | chain C |
| | | | | | | | region |
| L9.2.2.322 | 20 | GCCTGCCGGGACTG | agg | normal | mRNA | NM_001277457 | Gallus gallus |
| | | GGCTGC(SEQ ID | | | | | ribosomal |
| | | NO: 260) | | | | | protein L35a |
| | | | | | | | (RPL35A), |
| | | | | | | | mRNA |
| L9.2.2.323 | 20 | TGCAAAAAACCAGG | tgg | normal | mRNA | NM_001277663 | Gallus gallus |
| | | CTGGAC(SEQ ID | | | | | ribosomal |
| | | NO: 261) | | | | | protein L27a |
| | | | | | | | (RPL27A), |
| | | | | | | | mRNA |
| L9.2.2.324 | 19 | CATGATTAAGAGGG | cgg | normal | rRNA | HQ873432 | Gallus gallus |
| | | ACGGC(SEQ ID | | | | | isolate ML48 |
| | | NO: 262) | | | | | 18S |
| | | | | | | | ribosomal |
| | | | | | | | RNA gene, |
| | | | | | | | partial |
| | | | | | | | sequence |
| L9.2.2.325 | 21 | GGGAGCGGCGGCCGT | | | | | |
| | | GGCGGC(SEQ | | | | | |
| | | ID NO: 263) | | | | | |
| L9.2.2.326 | 19 | TCGGTGAAGTCCC | | | | | |
| | | CAAAAT(SEQ | | | | | |
| | | ID NO: 264) | | | | | |
| L9.2.2.327 | 20 | TCGACGATGGCACG | cgg | normal | mRNA | NM_205337 | Gallus gallus |
| | | TCTGAT(SEQ ID | | | | | ribosomal |
| | | NO: 265) | | | | | protein L27 |
| | | | | | | | (RPL27), |
| | | | | | | | mRNA |
| L9.2.2.328 | 20 | CCGTCCCGCGAGGA | agg | normal | mRNA | X01613 | Gallus gallus |
| (Cμ | | CTTCGA(SEQ ID | | | | | mRNA for |
| guide | | NO: 266) | | | | | mu |
| 1) | | | | | | | immunoglobulin |
| | | | | | | | heavy |
| | | | | | | | chain C |
| | | | | | | | region |
| L9.2.2.329 | 20 | AACATCTCTCCCTT | tgg | normal | mRNA | NM_204987 | Gallus gallus |
| | | CTCCTT(SEQ ID | | | | | ribosomal |
| | | NO: 267) | | | | | protein, large, |
| | | | | | | | P0 (RPLP0), |
| | | | | | | | mRNA |
| L9.2.2.330 | 20 | GAGGAAGACACCGT | cgg | normal | mRNA | NM_001005823 | Gallus gallus |
| | | CCCCAC(SEQ ID | | | | | small nuclear |
| | | NO: 268) | | | | | ribonucleoprotein |
| | | | | | | | Polypeptide |
| | | | | | | | A′ |
| | | | | | | | (SNRPA1), |
| | | | | | | | mRNA |
| L9.2.2.331 | 20 | CCCGCCCGCGCTCC | cgg | normal | mRNA | NM_001113741 | Gallus gallus |
| | | GCGCAC(SEQ ID | | | | | serine/arginine- |
| | | NO: 269) | | | | | rich |
| | | | | | | | splicing |
| | | | | | | | factor 1 |
| | | | | | | | (SRSF1), |
| | | | | | | | mRNA |
| L9.2.2.332 | 20 | CGCCTGTGTGATTACTCTAT | | | | | |
| | | (SEQ ID NO: 270) | | | | | |
| L9.2.2.333 | 20 | GGCGCTCTTCCGGG | tgg | reverse | mRNA | XM_415820 | PREDICTED: |
| | | GGTATT(SEQ ID | | | | | Gallus |
| | | NO: 271) | | | | | gallus |
| | | | | | | | ribosomal |
| | | | | | | | protein L23a |
| | | | | | | | (RPL23A), |
| | | | | | | | mRNA |
| L9.2.2.334 | 20 | GACTAACATTCCTC | agg | normal | mRNA | XM_414630 | PREDICTED: |
| | | AAACCC(SEQ ID | | | | | Gallus |
| | | NO: 272) | | | | | gallus SEC24 |
| | | | | | | | family, |
| | | | | | | | member A (S. cerevisiae) |
| | | | | | | | (SEC24A), |
| | | | | | | | transcript |
| | | | | | | | variant X2, |
| | | | | | | | mRNA |
| L9.2.2.335 | 20 | CGTTCCGAAGGGAC | tgg | normal | rRNA | JN639848 | Gallus gallus |
| | | GGGCGA(SEQ ID | | | | | 28S |
| | | NO: 273) | | | | | ribosomal |
| | | | | | | | RNA, partial |
| | | | | | | | sequence |
| L9.2.2.336 | 20 | GGCGGAAGCAGCGA | agg | | | | |
| | | ACAGAG (SEQ ID | | | | | |
| | | NO: 274) | | | | | |
| L9.2.2.337 | 20 | CCAAAGCCAATCGG | cgg | normal | mRNA | X01613 | Gallus gallus |
| (Cμ | | TCACAT (SEQ ID | | | | | mRNA for |
| guide | | NO: 275) | | | | | mu |
| 2) | | | | | | | immunoglobulin |
| | | | | | | | heavy |
| | | | | | | | chain C |
| | | | | | | | region |
| L9.2.2.338 | 20 | CCGTTAAGAGGTAA | ggg | reverse | rRNA | DQ018756 | Gallus gallus |
| | | ACGGGT (SEQ ID | | | | | 28S |
| | | NO: 276) | | | | | ribosomal |
| | | | | | | | RNA gene, |
| | | | | | | | partial |
| | | | | | | | sequence |
| L9.2.2.339 | 20 | ATGCATGTCTAAGT | ggg | normal | rRNA | HQ873432 | Gallus gallus |
| | | ACACAC (SEQ ID | | | | | isolate ML48 |
| | | NO: 277) | | | | | 18S |
| | | | | | | | ribosomal |
| | | | | | | | RNA gene, |
| | | | | | | | partial |
| | | | | | | | sequence |
| L9.2.2.340 | 20 | TCCGGCAAGTCCAC | cgg | normal | mRNA | AY579777 | Gallus gallus |
| | | CACCAC (SEQ ID | | | | | elongation |
| | | NO: 278) | | | | | factor 1 alpha |
| | | | | | | | (EF1A) gene, |
| | | | | | | | partial cds |
| L9.2.2.341 | 20 | TCCGCACCGCCGGC | cgg | reverse | rRNA | FM165415 | Gallus gallus |
| | | GACGGC (SEQ ID | | | | | 28S rRNA |
| | | NO: 279) | | | | | gene, clone |
| | | | | | | | GgLSU-1 |
| L9.2.2.342 | 20 | CGTTCCCTCCGCTT | cgg | normal | mRNA | NM_001031373 | Gallus gallus |
| | | CGACCC (SEQ ID | | | | | ubiquilin 4 |
| | | NO: 280) | | | | | (UBQLN4), |
| | | | | | | | mRNA |
| L9.2.2.343 | 20 | TGGACCCCTACAGTATGTTC | | | | | |
| | | (SEQ ID NO: 281) | | | | | |
| L9.2.2.344 | 20 | CGAATACAGACCGT | ggg | normal | mRNA | AB556518 | Gallus gallus |
| | | GAAAGC (SEQ ID | | | | | DNA, CENP- |
| | | NO: 282) | | | | | A associated |
| | | | | | | | sequence, |
| | | | | | | | partial |
| | | | | | | | sequence, |
| | | | | | | | clone: |
| | | | | | | | CAIP#220 |
| L9.2.2.345 | 20 | CATCGGGAAGAGAA | cgg | normal | mRNA | AY393838 | Gallus gallus |
| | | AGGGTA (SEQ ID | | | | | ribosomal |
| | | NO: 283) | | | | | protein L19 |
| | | | | | | | mRNA, |
| | | | | | | | partial cds |

### REFERENCES

1. R. Barrangou, C. Fremaux, H. Deveau, M. Richards, P. Boyaval, S. Moineau, D. A. Romero, P. Horvath, CRISPR provides acquired resistance against viruses in prokaryotes. Science 315, 1709-1712 (2007).
2. I. Grissa, G. Vergnaud, C. Pourcel, The CRISPRdb database and tools to display CRISPRs and to generate dictionaries of spacers and repeats. BMC Bioinformatics 8, 172 (2007).
3. J. E. Garneau, M. E. Dupuis, M. Villion, D. A. Romero, R. Barrangou, P. Boyaval, C. Fremaux, P. Horvath, A. H. Magadan, S. Moineau, The CRISPR/Cas bacterial immune system cleaves bacteriophage and plasmid DNA. Nature 468, 67-71 (2010).
4. L. Cong, F. A. Ran, D. Cox, S. Lin, R. Barretto, N. Habib, P. D. Hsu, X. Wu, W. Jiang, L. A. Marraffini, F. Zhang, Multiplex genome engineering using CRISPR/Cas systems. Science 339, 819-823 (2013).
5. P. Mali, L. Yang, K. M. Esvelt, J. Aach, M. Guell, J. E. DiCarlo, J. E. Norville, G. M. Church, RNA-guided human genome engineering via Cas9. Science 339, 823-826 (2013).
6. O. Shalem, N. E. Sanjana, E. Hartenian, X. Shi, D. A. Scott, T. S. Mikkelsen, D. Heckl, B. L. Ebert, D. E. Root, J. G. Doench, F. Zhang, Genome-scale CRISPR-Cas9 knockout screening in human cells. Science 343, 84-87 (2014).
7. T. Wang, J. J. Wei, D. M. Sabatini, E. S. Lander, Genetic screens in human cells using the CRISPR-Cas9 system. Science 343, 80-84 (2014).
8. Y. Zhou, S. Zhu, C. Cai, P. Yuan, C. Li, Y. Huang, W. Wei, High-throughput screening of a CRISPR/Cas9 library for functional genomics in human cells. Nature 509, 487-491 (2014).
9. H. Koike-Yusa, Y. Li, E. P. Tan, C. Velasco-Herrera Mdel, K. Yusa, Genome-wide recessive genetic screening in mammalian cells with a lentiviral CRISPR-guide RNA library. Nat. Biotechnol. 32, 267-273 (2014).
10. A. Meisel, T. A. Bickle, D. H. Kruiger, C. Schroeder, Type III restriction enzymes need two inversely oriented recognition sites for DNA cleavage. Nature 355, 467-469 (1992).
11. H. Arakawa, D. Lodygin, J. M. Buerstedde, Mutant loxP vectors for selectable marker recycle and conditional knock-outs. BMC Biotechnol. 1, 7 (2001).
12. H. Arakawa, J. Hauschild, J. M. Buerstedde, Requirement of the activation-induced deaminase (AID) gene for immunoglobulin gene conversion. Science 295, 1301-1306 (2002).
13. Y. Y. Zhu, E. M. Machleder, A. Chenchik, R. Li, P. D. Siebert, Reverse Transcriptase template switching: A SMART approach for full-length cDNA Library Construction. BioTechniques 30, 892-897 (2001).
14. S. Lundin, A. Jemt, F. Terje-Hegge, N. Foam, E. Pettersson, M. Killer, V. Wirta, P. Lexow, J. Lundeberg, Endonuclease specificity and sequence dependence of type IIS restriction enzymes. PLoS One 10, e0117059 (2015).
15. N. E. Sanjana, O. Shalem, F. Zhang, Improved vectors and genome-wide libraries for CRISPR screening. Nat. Methods 11, 783-784 (2014).
16. International Chicken Genome Sequencing Consortium, Sequence and comparative analysis of the chicken genome provide unique perspectives on vertebrate evolution. Nature 432, 695-716 (2004).
17. J. Cheng et al., A Molecular Chipper technology for CRISPR sgRNA library generation and functional mapping of noncoding regions. Nat. Commun. 7, 11178 (2016).
18. A. B. Lane et al., Enzymatically Generated CRISPR Libraries for Genome Labeling and Screening. Dev. Cell. 34, 373-378 (2015).
19. S. R. Patanjali, S. Parimoo, S. M. Weissman, Construction of a uniform-abundance (normalized) cDNA library. Proc Natl Acad Sci US A. 88, 1943-1947 (1991).
20. C. A. Reynaud, V. Anquez, H. Grimal, J. C. Weill, A hyperconversion mechanism generates the chicken light chain preimmune repertoire. Cell 48, 379-388 (1987).
21. J. M. Buerstedde, S. Takeda, Increased ratio of targeted to random integration after transfection of chicken B cell lines. Cell 67, 179-188 (1991).
22. Y. Umesono, J. Tasaki, Y. Nishimura, M. Hrouda, E. Kawaguchi, S. Yazawa, O. Nishimura, K. Hosoda, T. Inoue, K. Agata, The molecular logic for planarian regeneration along the anterior-posterior axis. Nature 500, 73-76 (2013).
23. X. Tian, J. Azpurua, C. Hine, A. Vaidya, M. Myakishev-Rempel, J. Ablaeva, Z. Mao, E. Nevo, V. Gorbunova, A. Seluanov, High-molecular-mass hyaluronan mediates the cancer resistance of the naked mole rat. Nature 499, 346-349 (2013).
24. T. A. Ebert, J. R. Southon, Red sea urchins (Strongylocentrotus franciscanus) can live over 100 years: confirmation with A-bomb 14Carbon. Fish. Bull. 101, 915-922 (2003).
25. S. A. Stewart, D. M. Dykxhoorn, D. Palliser, H. Mizuno, E. Y. Yu, D. S. An, D. M. Sabatini, I. S. Chen, W. C. Hahn, P. A. Sharp, R. A. Weinberg, C. D. Novina, Lentivirus-delivered stable gene silencing by RNAi in primary cells. RNA 9. 493-501 (2003).

1. A method to produce a clustered regularly interspersed short palindromic repeats (CRISPR)-Cas single-guide RNA (sgRNA) library or a sgRNA or a guide sequence, comprising synthesizing cDNA from an MRNA sequence with a semi-random primer comprising a protospacer adjacent motif (PAM)-complementary sequence as cDNA synthesis primer.

2. The method according to claim 1, wherein said semi-random primer is 4 to 10 nucleotides long.

3. The method according to claim 1 wherein the PAM-complementary sequence is complementary to a PAM sequence specific for S. progenies (Sp) Cas9, Neisseria meningitidis (NM) Cas9, Streptococcus thermophilus (ST) Cas9 or Treponema denticola (TD) Cas9, orthologues, homologues or variants thereof.

4. The method according to claim 1, wherein the PAM sequence is selected from the group consisting of: 5′-NGG-3′, 5′-NNNNGATT-3′, 5′-NNAGAAW-3′ and 5′-NAAAAC-3′, orthologues, homologues or variants thereof, wherein N is a nucleotide selected from C, G, A and T.

5. The method according to claim 1 wherein the PAM-complementary sequence comprises the sequence 5-CCN-3′, wherein N is a nucleotide selected from C, G, A and T, said primer being preferably phosphorylated at the 5′ terminus.

6. The method according to claim 1 wherein the semi-random primer comprises or has essentially the sequence of SEQ ID NO: 1 (5′-NNNCCN-3′).

7. Method for obtaining a guide sequence comprising the following steps:
a) synthesizing DNA from a RNA or a DNA using a semi-random primer as defined in claim 1, and
b) generating guide sequences by molecular biological methods.

8. The method according to claim 7, wherein the guide sequence is generated by cutting the synthetized DNA to obtain a guide sequence.

9. The method according to claim 7 wherein the obtained guide sequence consists of 20 base pairs.

10. The method according to claim 7 wherein the cutting is carried out with a type III restriction enzyme and/or a type IIS restriction enzyme.

11. The method according to claim 7 wherein the cutting is carried out with enzymes that cleave 25/27 and/or 14/16 base pairs away from their recognition site.

12. The method according to claim 7 wherein the method further comprises, before cutting the synthetized DNA, a step wherein the synthetized DNA is modified by addition of restriction sites for said restriction enzymes.

13. The method according to claim 7, wherein step b) comprises the following steps:
i) modification of synthetized DNA by addition:
to the 5′ end of the synthetized DNA of a linker sequence comprising a type III first restriction site and/or a type IIS second restriction site
and/or
to the 3′ end of the synthetized DNA of a linker sequence comprising a type IIS third restriction site and/or a type III fourth restriction sites, and
ii) cutting of the modified DNA.

14. The method according to claim 7, wherein the synthetized DNA is a dsDNA.

15. The method according to claim 7, wherein the RNA is a mRNA.

16. The method according to claim 7, wherein the type III restriction site is a EcoP151 restriction site.

17. The method according to claim 7 wherein the type IIS restriction site is a AcuI restriction site.

18. The method according to claim 7, wherein the linker sequence at the 5′ end of the synthetized DNA further comprises a fifth restriction site, and/or the linker sequence at the 3′ end of the synthetized DNA further comprises a sixth restriction site.

19. The method according to claim 7, further comprising a step i′) wherein the modified DNA is digested with the specific type III restriction enzyme.

20. The method according to claim 19, further comprising a step i″) wherein the to the 5′ end of the digested DNA is added a further linker sequence comprising a seventh restriction site which is a cloning site for the gRNA expression vector and a eight restriction site, and the DNA is then optionally digested with the specific restriction enzyme for the fifth restriction site at the 5′.

21. The method according to claim 20, further comprising a step i′″) wherein the DNA is amplified, and digested with the specific type IIS restriction enzyme for the third restriction site at the 3′ and optionally with the specific restriction enzyme for the sixth restriction site.

22. The method according to claim 21, further comprising a step i″″) wherein the guide sequence fragment is purified from the digested DNA and ligated with a further linker sequence at the 3′ end comprising a restriction site which is a cloning site for the gRNA expression vector and optionally a ninth restriction site.

23. The method according to claim 22, further comprising a step i′″″) wherein the DNA is amplified, and digested with the specific restriction enzyme for the cloning site and optionally with the specific restriction enzyme for the ninth restriction site.

24. The method according to claim 7, wherein 25-bp fragments are purified.

25. An isolated guide sequence obtainable by the method of claim 7.

26. An isolated sgRNA comprising the RNA corresponding to the isolated guide sequence according to claim 25.

27. Method for obtaining a CRISPR-Cas system sgRNA library comprising cloning the guide sequences of claim 25 into a sgRNA expression vector and transforming said vector into a competent cell to obtain a CRISP-Cas system sgRNA library.

28. The method according to claim 27 wherein the expression vector is a lentivirus, and/or the vector comprises a species specific functional promoter and/or a gRNA scaffold sequence.

29. A CRISPR-Cas system sgRNA library obtainable by the method of claim 27.

30. A library comprising a plurality of CRISPR-Cas system guide sequences that target a plurality of target sequences in genomic loci of a plurality of genes, wherein said targeting results in a knockout of gene function,
wherein the unique CRISPR-Cas system guide sequences are obtained by using a semi-random primer as defined in claim 1.

31. The library of claim 29 wherein the plurality of genes are Gallus gallus genes.

32. An isolated sgRNA or an isolated guide sequence selected from the library of claim 29.

33. (canceled)

34. A kit comprising a semi-random primer for carrying out the method of claim 7.

35. (canceled)

36. A kit comprising one or more vectors, each vector comprising at least one guide sequence according to claim 25, wherein the vector comprises a first regulatory element operably linked to a tracr mate sequence and a guide sequence upstream of the tracr mate sequence, wherein when expressed, the guide sequence directs sequence-specific binding of a CRISPR complex to a target sequence in a eukaryotic cell, wherein the CRISPR complex comprises a Cas9 enzyme complexed with (1) the guide sequence and (2) the tracr mate sequence that is hybridized to a tracr sequence.

37. An isolated DNA molecule encoding the guide sequence according to claim 25.

38. A vector comprising a DNA molecule according to claim 37.

39. An isolated host cell comprising a DNA molecule according to claim 37.

40. The isolated host cell which has been transduced with the library of claim 29.


SEQUENCE LISTING (284 sequences)

SEQ ID NO: 1 - DNA, 6 bp, Artificial Sequence
  semi-random primer
  misc_feature (1)..(3): n is a, c, g, or t
  misc_feature (6)..(6): n is a, c, g, or t
nnnccn

SEQ ID NO: 2 - DNA, 32 bp, Artificial Sequence
  3′ linker I
ctgctgacttcagtggttctagaggtgtccaa

SEQ ID NO: 3 - DNA, 34 bp, Artificial Sequence
  3′ linker I
gttggacacctctagaaccactgaagtcagcagt

SEQ ID NO: 4 - DNA, 75 bp, Artificial Sequence
  u6 promoter + guide sequence + gRNA scaffold
  misc_feature (29)..(48): n is a, c, g, t or u
tatatcttgtggaaaggacgaaacaccgnnnnnnnnnnnnnnnnnnnngttttagagctagaaatagcaagttaa

SEQ ID NO: 5 - DNA, 60 bp, Artificial Sequence
  U6 promoter + guide sequence + gRNA scaffolg
tatatatcttgtggaaaggacgaaacaccggttttagagctagaaatagcaagttaaaat

SEQ ID NO: 6 - DNA, 80 bp, Artificial Sequence
  U6 promoter + guide sequence + gRNA scaffolg
tatatatcttgtggaaaggacgaaacaccgaacagcacccaccaccactggttttagagctagaaatagcaagttaaaat

SEQ ID NO: 7 - DNA, 80 bp, Artificial Sequence
  U6 promoter + guide sequence + gRNA scaffolg
tatatatcttgtggaaaggacgaaacaccgcgtcgccaagacctcgaggagttttagagctagaaatagcaagttaaaat

SEQ ID NO: 8 - DNA, 80 bp, Artificial Sequence
  U6 promoter + guide sequence + gRNA scaffolg
tatatatcttgtggaaaggacgaaacaccgtcgacgatggcacgtctgatgttttagagctagaaatagcaagttaaaat

SEQ ID NO: 9 - DNA, 80 bp, Artificial Sequence
  U6 promoter + guide sequence + gRNA scaffolg
tatatatcttgtggaaaggacgaaacaccggcgttgtgggggatcgtcgggttttagagctagaaatagcaagttaaaat

SEQ ID NO: 10 - DNA, 80 bp, Artificial Sequence
  U6 promoter + guide sequence + gRNA scaffolg
tatatatcttgtggaaaggacgaaacaccgaaggtggtgctggtgctcgcgttttagagctagaaatagcaagttaaaat

SEQ ID NO: 11 - DNA, 80 bp, Artificial Sequence
  U6 promoter + guide sequence + gRNA scaffolg
tatatatcttgtggaaaggacgaaacaccgcagcaccgtgctgacatttcgttttagagctagaaatagcaagttaaaat

SEQ ID NO: 12 - DNA, 80 bp, Artificial Sequence
  U6 promoter + guide sequence + gRNA scaffolg
tatatatcttgtggaaaggacgaaacaccgggcgctgagcagctgttcctgttttagagctagaaatagcaagttaaaat

SEQ ID NO: 13 - DNA, 80 bp, Artificial Sequence
  U6 promoter + guide sequence + gRNA scaffolg
tatatatcttgtggaaaggacgaaacaccggataggcacaatcttttcacgttttagagctagaaatagcaagttaaaat

SEQ ID NO: 14 - DNA, 80 bp, Artificial Sequence
  U6 promoter + guide sequence + gRNA scaffolg
tatatatcttgtggaaaggacgaaacaccgacctccaagaccggcaagcagttttagagctagaaatagcaagttaaaat

SEQ ID NO: 15 - DNA, 80 bp, Artificial Sequence
  U6 promoter + guide sequence + gRNA scaffolg
tatatatcttgtggaaaggacgaaacaccgcagtcgctcttggcattctcgttttagagctagaaatagcaagttaaaat

SEQ ID NO: 16 - DNA, 80 bp, Artificial Sequence
  U6 promoter + guide sequence + gRNA scaffolg
tatatatcttgtggaaaggacgaaacaccggtccgagaaagcaccttccagttttagagctagaaatagcaagttaaaat

SEQ ID NO: 17 - DNA, 80 bp, Artificial Sequence
  U6 promoter + guide sequence + gRNA scaffolg
tatatatcttgtggaaaggacgaaacaccgccctcttatccaggacctacgttttagagctagaaatagcaagttaaaat

SEQ ID NO: 18 - DNA, 20 bp, Gallus gallus
aacagcacccaccaccactg

SEQ ID NO: 19 - DNA, 20 bp, Gallus gallus
aacagcacccaccaccactg

SEQ ID NO: 20 - DNA, 300 bp, Gallus gallus
agcacggcacccacctttgctgccccagtgttccagtttggaaagccggctccagccaccgtctctgccactgccagcgtcacgggaggcccagcgtttggccaagcacctgcaaactcaacagcacccaccaccactgcgggcttcagcatatttgggagcaccacgttgacatcttctgccccggccaccgcaggccaaccggcgctgacgtttggctcctccacttcagcttttggcggtactttcagcacaagtgtgaagccactgccgccgtactcaggggcagcgagccagccc

SEQ ID NO: 21 - DNA, 1398 bp, Gallus gallus
gcctcggcctccccgtcgcccccccgcctcttcccgttggttctgtgctccccctccgactccgtctacaccgtcggctgcgccgccttcgacttccagccctcctccatcgccttcacgtggttcgattccaacaacagttccgtttccggtatggatgttatccctaaagtcatttccggtccaccttaccgggccgtcagtcgaatacagatgaatcaaagcgaagggaaagagaaacagcccttccggtgtcgggcggcgcatccacgcggcaacgtcgaggtcagcgtgatgaacccaggcccgattcccaccccgaatggcatcccccttttcgtcaccatgcaccccccgtcccgcgaggacttcgaaggccccttccgcaacgcctccatcctctgccagacccgcgggcgccgccgtcccaccgaggtcacgtggtacaaaaatggcagccccgtcgccgccgccgccaccaccgccaccaccgtcggccccgaagtggtggccgagagccgcatcagcgtcaccgaaagcgaatgggacaccggggccaccttcagctgcgtcgtggagggggagatgaggaacaccagcaagaggatggagtgcggattagaacccgtcgtgcagcaggacatcgccatccgcgtcatcacgccgtccttcgtggacatcttcatcagcaaatcggccacgctgacgtgccgggtgagcaacatggtgaacgccgacggcctggaggtgtcgtggtggaaggagaaggggggcaaactggagacggcgttggggaagagggtcctgcaaagcaacggcctctacacggtggacggggtggccacggtgtgcgccagcgaatgggacggaggggatggctacgtgtgtaaggtgaaccaccccgatctgctcttccccatggaggagaagatgaggaagacgaaagccagcaacgcccgccccccatccgtctacgtcttccccccccccacggaacaactgaacggcaaccaacggctcagcgtcacctgcatggctcagggcttcaaccccccccacctcttcgtcaggtggatgagaaacggggaacccctcccccaaagccaatcggtcacatcggcccccatggcggagaaccccgaaaatgagtcctacgtggcctacagcgttttgggggtgggggccgaagagtggggcgccggcaacgtctacacgtgcctggtgggccacgaagctctgcccctccagctggcccagaagtcggtggatagggcttcgggtaaagcaagtgctgtcaatgtctccttggtgttggccgactcggccgccgcctgctattaattaattaacccgctcgttaagcggccgctcgattgggattaaagagcagatgtccat

SEQ ID NO: 22 - DNA, 33 bp, Gallus gallus
accccccgtcccgcgaggacttcgaaggcccct

SEQ ID NO: 23 - DNA, 33 bp, Gallus gallus
ctcccccaaagccaatcggtcacatcggccccc

SEQ ID NO: 24 - DNA, 32 bp, Gallus gallus
tcggcccccatggcggagaaccccgaaaatga

SEQ ID NO: 25 - DNA, 40 bp, Gallus gallus
ctcctggccgccctgccaggttagaacccgtcgtgcagca

SEQ ID NO: 26 - DNA, 40 bp, Gallus gallus
gctcagcctcgtctgcaaggcaacggcctctacacggtgg

SEQ ID NO: 27 - DNA, 40 bp, Gallus gallus
gtctcctccgcctcggcctcaagacgaaagccagcaacgc

SEQ ID NO: 28 - DNA, 40 bp, Gallus gallus
tgatgaacccaggtcccccgcggggaacccctcccccaaa

SEQ ID NO: 29 - DNA, 40 bp, Artificial Sequence
  5′ SMART tag
tggtcaagcttcagcagatctacacggacgtcgcrgrgrg

SEQ ID NO: 30 - DNA, 26 bp, Artificial Sequence
  5′ SMART PCR primer
tggtcaagcttcagcagatctacacg

SEQ ID NO: 31 - DNA, 32 bp, Artificial Sequence; identical to SEQ ID NO: 2
  3′ linker forward

SEQ ID NO: 32 - DNA, 34 bp, Artificial Sequence; identical to SEQ ID NO: 3
  3′ linker reverse

SEQ ID NO: 33 - DNA, 26 bp, Artificial Sequence
  5′ linker I forward
gcatataagcttgacgtctctcaccg

SEQ ID NO: 34 - DNA, 28 bp, Artificial Sequence
  5′ linker I reverse
  misc_feature (1)..(2): n is a, c, g, t or u
nncggtgagagacgtcaagcttatatgc

SEQ ID NO: 35 - DNA, 26 bp, Artificial Sequence
  3′ linker II forward
gtttggagacgtcttctagatcagcg

SEQ ID NO: 36 - DNA, 28 bp, Artificial Sequence
  3′ linker II reverse
  misc_feature (27)..(28): n is a, c, g, t or u
cgctgatctagaagacgtctccaaacnn

SEQ ID NO: 37 - DNA, 39 bp, Artificial Sequence
  3′ linker I PCR primer
  misc_feature (35)..(37): n is a, c, g, t or u
gttggacacctctagaaccactgaagtcagcagtnnncc

SEQ ID NO: 38 - DNA, 26 bp, Artificial Sequence
  3′ linker II PCR primer
cgctgatctagaagacgtctccaaac

SEQ ID NO: 39 - DNA, 27 bp, Artificial Sequence
  Sequencing primer
ttttcgggtttattacagggacagcag

SEQ ID NO: 40 - DNA, 30 bp, Artificial Sequence
  lentiCRISPR forward
cttggctttatatatcttgtggaaaggacg

SEQ ID NO: 41 - DNA, 33 bp, Artificial Sequence
  lentiCRISPR reverse
cggactagccttattttaacttgctatttctag

SEQ ID NO: 42 - DNA, 24 bp, Artificial Sequence
  universal forward
agcggataacaatttcacacagga

SEQ ID NO: 43 - DNA, 24 bp, Artificial Sequence
  universal reverse
cgccagggttttcccagtcacgac

SEQ ID NO: 44 - DNA, 39 bp, Artificial Sequence
  Ig heavy chain 1
ccgcaaccaagcttatgagcccactcgtctcctccctcc

SEQ ID NO: 45 - DNA, 42 bp, Artificial Sequence
  Ig heavy chain 2
cgtccatctagaatggacatctgctctttaatcccaatcgag

SEQ ID NO: 46 - DNA, 28 bp, Artificial Sequence
  Ig heavy chain 3
gctgaacaacctcagggctgaggacacc

SEQ ID NO: 47 - DNA, 32 bp, Artificial Sequence
  Ig heavy chain 4
agcaacgcccgccccccatccgtctacgtctt

SEQ ID NO: 48 - DNA, 20 bp, Artificial Sequence
  Guide Sequence
aacagcacccaccaccactg

SEQ ID NO: 49 - DNA, 20 bp, Artificial Sequence
  Guide Sequence
cgtcgccaagacctcgagga

SEQ ID NO: 50 - DNA, 20 bp, Artificial Sequence
  Guide Sequence
tcgacgatggcacgtctgat

SEQ ID NO: 51 - DNA, 20 bp, Artificial Sequence
  Guide Sequence
gcgttgtgggggatcgtcgg

SEQ ID NO: 52 - DNA, 20 bp, Artificial Sequence
  Guide Sequence
aaggtggtgctggtgctcgc

SEQ ID NO: 53 - DNA, 20 bp, Artificial Sequence
  Guide Sequence
cagcaccgtgctgacatttc

SEQ ID NO: 54 - DNA, 20 bp, Artificial Sequence
  Guide Sequence
ggcgctgagcagctgttcct

SEQ ID NO: 55 - DNA, 20 bp, Artificial Sequence
  Guide Sequence
gataggcacaatcttttcac

SEQ ID NO: 56 - DNA, 20 bp, Artificial Sequence
  Guide Sequence
acctccaagaccggcaagca

SEQ ID NO: 57 - DNA, 20 bp, Artificial Sequence
  Guide Sequence
cagtcgctcttggcattctc

SEQ ID NO: 58 - DNA, 20 bp, Artificial Sequence
  Guide Sequence
gtccgagaaagcaccttcca

SEQ ID NO: 59 - DNA, 20 bp, Artificial Sequence
  Guide Sequence
ccctcttatccaggacctac

SEQ ID NO: 60 - DNA, 20 bp, Artificial Sequence
  Guide Sequence
tgctggggttcgtgtgtgtc

SEQ ID NO: 61 - DNA, 20 bp, Artificial Sequence
  Guide Sequence
ggggtcgtcgaaggacacgg

SEQ ID NO: 62 - DNA, 20 bp, Artificial Sequence
  Guide Sequence
tattaaattaaagctcgtcc

SEQ ID NO: 63 - DNA, 19 bp, Artificial Sequence
  Guide Sequence
cgaatacagaccgtgaaag

SEQ ID NO: 64 - DNA, 20 bp, Artificial Sequence
  Guide Sequence
cccgtgaaaatccgggggag

SEQ ID NO: 65 - DNA, 19 bp, Artificial Sequence
  Guide Sequence
tgtattttgaagacaacgc

SEQ ID NO: 66 - DNA, 20 bp, Artificial Sequence
  Guide Sequence
ccctgctacgctgccttgtt

SEQ ID NO: 67 - DNA, 20 bp, Artificial Sequence
  Guide Sequence
cgcgatgagggaacttccgc

SEQ ID NO: 68 - DNA, 20 bp, Artificial Sequence
  Guide Sequence
cagtgcctgcaggaccctcc

SEQ ID NO: 69 - DNA, 19 bp, Artificial Sequence
  Guide Sequence
catgattaagagggacggc

SEQ ID NO: 70 - DNA, 20 bp, Artificial Sequence
  Guide Sequence
ccgcagcgaccgcacgtccc

SEQ ID NO: 71 - DNA, 20 bp, Artificial Sequence
  Guide Sequence
cgcggttttcgtccaataaa

SEQ ID NO: 72 - DNA, 19 bp, Artificial Sequence
  Guide Sequence
tcctgtccatggccaacgc

SEQ ID NO: 73 - DNA, 20 bp, Artificial Sequence
  Guide Sequence
gcccgcagccgatcctccgc

SEQ ID NO: 74 - DNA, 19 bp, Artificial Sequence
  Guide Sequence
tctgtatcttccttcacat

SEQ ID NO: 75 - DNA, 20 bp, Artificial Sequence
  Guide Sequence
cgtccacctttgctttcttc

SEQ ID NO: 76 - DNA, 20 bp, Artificial Sequence
  Guide Sequence
cgaggaattcccagtaagtg

SEQ ID NO: 77 - DNA, 19 bp, Artificial Sequence
  Guide Sequence
ttttgttggttttcggaaa

SEQ ID NO: 78 - DNA, 20 bp, Artificial Sequence
  Guide Sequence
ggcccccaagatcggaccgc

SEQ ID NO: 79 - DNA, 20 bp, Artificial Sequence
  Guide Sequence
cggctccgggacggctggga

SEQ ID NO: 80 - DNA, 20 bp, Artificial Sequence
  Guide Sequence
cgcagcatttatgggcacag

SEQ ID NO: 81 - DNA, 20 bp, Artificial Sequence
  Guide Sequence
gggataaggattggctctaa

SEQ ID NO: 82 - DNA, 20 bp, Artificial Sequence
  Guide Sequence
tcctagagcaaggcaaacgt

SEQ ID NO: 83 - DNA, 20 bp, Artificial Sequence
  Guide Sequence
aacccgactccgagaagccc

SEQ ID NO: 84 - DNA, 20 bp, Artificial Sequence
  Guide Sequence
gcgccgccaccttccgcaac

SEQ ID NO: 85 - DNA, 20 bp, Artificial Sequence
  Guide Sequence
gcggggagcatggcggagag

SEQ ID NO: 86 - DNA, 20 bp, Artificial Sequence
  Guide Sequence
gggtgcgtttgggaagccgc

SEQ ID NO: 87 - DNA, 20 bp, Artificial Sequence
  Guide Sequence
ggtttttttccttagccaag

SEQ ID NO: 88 - DNA, 20 bp, Artificial Sequence
  Guide Sequence
cgcttccggcgtcttgcgcc

SEQ ID NO: 89 - DNA, 20 bp, Artificial Sequence
  Guide Sequence
ccccgcctccgcctcccctc

SEQ ID NO: 90 - DNA, 20 bp, Artificial Sequence
  Guide Sequence
cagccacagggcacagtgag

SEQ ID NO: 91 - DNA, 20 bp, Artificial Sequence
  Guide Sequence
gctgaagaacatgagcacgg

SEQ ID NO: 92 - DNA, 20 bp, Artificial Sequence
  Guide Sequence
tccccggcgccgctctcggg

SEQ ID NO: 93 - DNA, 20 bp, Artificial Sequence
  Guide Sequence
agcataccaatcagctacgc

SEQ ID NO: 94 - DNA, 20 bp, Artificial Sequence
  Guide Sequence
tcctgttggctgaggctcgt

SEQ ID NO: 95 - DNA, 20 bp, Artificial Sequence
  Guide Sequence
ggggacgtaggagcgtatcg

SEQ ID NO: 96 - DNA, 20 bp, Artificial Sequence
  Guide Sequence
aacccagggggcaactttga

SEQ ID NO: 97 - DNA, 20 bp, Artificial Sequence
  Guide Sequence
ctaaccctcctctccctagc

SEQ ID NO: 98 - DNA, 20 bp, Artificial Sequence
  Guide Sequence
ggtcgggctggggcgcgaag

SEQ ID NO: 99 - DNA, 21 bp, Artificial Sequence
  Guide Sequence
tggcacttgcggaagcttccg

SEQ ID NO: 100 - DNA, 20 bp, Artificial Sequence
  Guide Sequence
cccacccgtgtgaccccgaa

SEQ ID NO: 101 - DNA, 17 bp, Artificial Sequence
  Guide Sequence
gattgagatttgggtgt

SEQ ID NO: 102 - DNA, 20 bp, Artificial Sequence
  Guide Sequence
ggcaaactcatgaaagctgg

SEQ ID NO: 103 - DNA, 20 bp, Artificial Sequence
  Guide Sequence
ggggctggacacagggacgc

SEQ ID NO: 104 - DNA, 20 bp, Artificial Sequence
  Guide Sequence
agaaatgaaaatcgttgtag

SEQ ID NO: 105 - DNA, 20 bp, Artificial Sequence
  Guide Sequence
cggggcgtgggcaaccgctg

SEQ ID NO: 106 - DNA, 20 bp, Artificial Sequence
  Guide Sequence
tcccgacgacctcctgcaac

SEQ ID NO: 107 - DNA, 20 bp, Artificial Sequence
  Guide Sequence
gttgtggccatggtgtggga

SEQ ID NO: 108 - DNA, 20 bp, Artificial Sequence
  Guide Sequence
catggcccagttttgcaagt

SEQ ID NO: 109 - DNA, 20 bp, Artificial Sequence
  Guide Sequence
gacaggcggtgcgggctggg

SEQ ID NO: 110 - DNA, 20 bp, Artificial Sequence
  Guide Sequence
tgaagctggcacacaaatac

SEQ ID NO: 111 - DNA, 20 bp, Artificial Sequence
  Guide Sequence
tgcttgtgcagaccaagcgt

SEQ ID NO: 112 - DNA, 20 bp, Artificial Sequence
  Guide Sequence
tgaggggagcagcaataaaa

SEQ ID NO: 113 - DNA, 20 bp, Artificial Sequence
  Guide Sequence
tggagccaccccaggaaatt

SEQ ID NO: 114 - DNA, 20 bp, Artificial Sequence
  Guide Sequence
cgtcccctcgccaatgacac

SEQ ID NO: 115 - DNA, 20 bp, Artificial Sequence
  Guide Sequence
cgccggcccccccccaaacc

SEQ ID NO: 116 - DNA, 20 bp, Artificial Sequence
  Guide Sequence
tgccgatccctcccgtcaaa

SEQ ID NO: 117 - DNA, 20 bp, Artificial Sequence
  Guide Sequence
gcagcagcgctccgtgctcc

SEQ ID NO: 118 - DNA, 19 bp, Artificial Sequence
  Guide Sequence
tccacccacacataaaccc

SEQ ID NO: 119 - DNA, 20 bp, Artificial Sequence
  Guide Sequence
tcctcgggacacacccgctc

SEQ ID NO: 120 - DNA, 20 bp, Artificial Sequence
  Guide Sequence
tgccaaatacgcagaagaga

SEQ ID NO: 121 - DNA, 21 bp, Artificial Sequence
  Guide Sequence
aacaaaatgctgtcctgcgcc

SEQ ID NO: 122 - DNA, 20 bp, Artificial Sequence
  Guide Sequence
tccgcggccgccgcagccat

SEQ ID NO: 123 - DNA, 19 bp, Artificial Sequence
  Guide Sequence
caggggaggcagatccaaa

SEQ ID NO: 124 - DNA, 20 bp, Artificial Sequence
  Guide Sequence
tggcacggggaaagcacgac

SEQ ID NO: 125 - DNA, 20 bp, Artificial Sequence
  Guide Sequence
ttgaaggccgaagtggagca

SEQ ID NO: 126 - DNA, 20 bp, Artificial Sequence
  Guide Sequence
caaacgtttgaagaggctgt

SEQ ID NO: 127 - DNA, 20 bp, Artificial Sequence
  Guide Sequence
tgcggagcaccgctcgtggt

SEQ ID NO: 128 - DNA, 18 bp, Artificial Sequence
  Guide Sequence
gtgcccatcccgcccaac

SEQ ID NO: 129 - DNA, 20 bp, Artificial Sequence
  Guide Sequence
cggccctgcgtcaggtacac

SEQ ID NO: 130 - DNA, 20 bp, Artificial Sequence
  Guide Sequence
tctgatgatgacatgggatt

SEQ ID NO: 131 - DNA, 20 bp, Artificial Sequence
  Guide Sequence
gggctctgagcagcctgagc

SEQ ID NO: 132 - DNA, 20 bp, Artificial Sequence
  Guide Sequence
catcgagctggtcatgtccc

SEQ ID NO: 133 - DNA, 20 bp, Artificial Sequence
  Guide Sequence
aatggtgcaaccgctattaa

SEQ ID NO: 134 - DNA, 20 bp, Artificial Sequence
  Guide Sequence
tccgtgctgctgggcggcga

SEQ ID NO: 135 - DNA, 20 bp, Artificial Sequence
  Guide Sequence
ggccgggactgcgcgcacag

SEQ ID NO: 136 - DNA, 20 bp, Artificial Sequence
  Guide Sequence
ctggtgaagtacatgaactc

SEQ ID NO: 137 - DNA, 20 bp, Artificial Sequence
  Guide Sequence
tgactagtcccacttataat

SEQ ID NO: 138 - DNA, 20 bp, Artificial Sequence
  Guide Sequence
ccgccgcctcccgcccctat

SEQ ID NO: 139 - DNA, 20 bp, Artificial Sequence
  Guide Sequence
tccctagcattcgagacaac

SEQ ID NO: 140 - DNA, 20 bp, Artificial Sequence
  Guide Sequence
ccacatggagcagccagcct

SEQ ID NO: 141 - DNA, 19 bp, Artificial Sequence
  Guide Sequence
ttctaaaacctttgtgcac

SEQ ID NO: 142 - DNA, 20 bp, Artificial Sequence
  Guide Sequence
ccgccacacacgcagagaac

SEQ ID NO: 143 - DNA, 19 bp, Artificial Sequence
  Guide Sequence
tttaacgaggatccattgg

SEQ ID NO: 144 - DNA, 20 bp, Artificial Sequence
  Guide Sequence
ccttcggagaggtgtcctcc

SEQ ID NO: 145 - DNA, 20 bp, Artificial Sequence
  Guide Sequence
ccctcagcgcgcccaaccgg

SEQ ID NO: 146 - DNA, 20 bp, Artificial Sequence
  Guide Sequence
cagccgccatgcctgccctc

SEQ ID NO: 147 - DNA, 20 bp, Artificial Sequence
  Guide Sequence
agaatagttttataaaccat

SEQ ID NO: 148 - DNA, 20 bp, Artificial Sequence
  Guide Sequence
ttttgttggttttcggaaac

SEQ ID NO: 149 - DNA, 20 bp, Artificial Sequence
  Guide Sequence
accctccgcggtaccctgaa

SEQ ID NO: 150 - DNA, 19 bp, Artificial Sequence
  Guide Sequence
tgagaatgagaagaacaat

SEQ ID NO: 151 - DNA, 20 bp, Artificial Sequence
  Guide Sequence
tgtagacaaaaactcagctc

SEQ ID NO: 152 - DNA, 21 bp, Artificial Sequence
  Guide Sequence
ggcccgatctggaatgaagat

SEQ ID NO: 153 - DNA, 20 bp, Artificial Sequence
  Guide Sequence
gcgagcggtgcggagaccac

SEQ ID NO: 154 - DNA, 20 bp, Artificial Sequence
  Guide Sequence
aagggcacagtgctgctgtc

SEQ ID NO: 155 - DNA, 20 bp, Artificial Sequence
  Guide Sequence
cgtggtggcctacctggtgc

SEQ ID NO: 156 - DNA, 20 bp, Artificial Sequence
  Guide Sequence
cagccttacaacatgtgatc

SEQ ID NO: 157 - DNA, 21 bp, Artificial Sequence
  Guide Sequence
catttccagccccatctgccc

SEQ ID NO: 158 - DNA, 20 bp, Artificial Sequence
  Guide Sequence
acgggccggtggtgcgcccg

SEQ ID NO: 159 - DNA, 20 bp, Artificial Sequence
  Guide Sequence
tccaaggcggggttgttctc

SEQ ID NO: 160 - DNA, 20 bp, Artificial Sequence
  Guide Sequence
cggcctcaacaaggctgaga

SEQ ID NO: 161 - DNA, 20 bp, Artificial Sequence
  Guide Sequence
acgggctgctgctgtgagca

SEQ ID NO: 162 - DNA, 20 bp, Artificial Sequence
  Guide Sequence
cgcctctcccccgcgggtgc

SEQ ID NO: 163 - DNA, 20 bp, Artificial Sequence
  Guide Sequence
tagctacccggcgtaaagag

SEQ ID NO: 164 - DNA, 20 bp, Artificial Sequence
  Guide Sequence
gggaccgccgttctacgttc

SEQ ID NO: 165 - DNA, 20 bp, Artificial Sequence
  Guide Sequence
ccatgattaagagggacggc

SEQ ID NO: 166 - DNA, 20 bp, Artificial Sequence
  Guide Sequence
cggcacgatgtttttaacgc

SEQ ID NO: 167 - DNA, 20 bp, Artificial Sequence
  Guide Sequence
ctgaggagcaggctaacaat

SEQ ID NO: 168 - DNA, 20 bp, Artificial Sequence
  Guide Sequence
ccgccgccaagggtaagaag

SEQ ID NO: 169 - DNA, 20 bp, Artificial Sequence
  Guide Sequence
caccttgcccagatcctgcc

SEQ ID NO: 170 - DNA, 20 bp, Artificial Sequence
  Guide Sequence
cgggggcacggagcacacat

SEQ ID NO: 171 - DNA, 20 bp, Artificial Sequence
  Guide Sequence
aacatctctcccttctcctt

SEQ ID NO: 172 - DNA, 20 bp, Artificial Sequence
  Guide Sequence
cgtcccggttcggcccggtc

SEQ ID NO: 173 - DNA, 20 bp, Artificial Sequence
  Guide Sequence
ctggtgaagtacatgaactc

SEQ ID NO: 174 - DNA, 20 bp, Artificial Sequence
  Guide Sequence
gcgcggccgtgctgccgagg

SEQ ID NO: 175 - DNA, 20 bp, Artificial Sequence
  Guide Sequence
cccaacccgggcatgctgtt

SEQ ID NO: 176 - DNA, 20 bp, Artificial Sequence
  Guide Sequence
cgtcgccaagacctcgagga

SEQ ID NO: 177 - DNA, 19 bp, Artificial Sequence
  Guide Sequence
ctttcaatgggtaagacgc

SEQ ID NO: 178 - DNA, 20 bp, Artificial Sequence
  Guide Sequence
aagtagtgctgcgaccagac

SEQ ID NO: 179 - DNA, 20 bp, Artificial Sequence
  Guide Sequence
gggttctgctctgcggcttc

SEQ ID NO: 180 - DNA, 20 bp, Artificial Sequence
  Guide Sequence
ggctcccctctgtgccccgc

SEQ ID NO: 181 - DNA, 20 bp, Artificial Sequence
  Guide Sequence
cggctccggggccggcgggg

SEQ ID NO: 182 - DNA, 20 bp, Artificial Sequence
  Guide Sequence
catggcgggaaccgcggcga

SEQ ID NO: 183 - DNA, 20 bp, Artificial Sequence
  Guide Sequence
gagtccattttggggggcgg

SEQ ID NO: 184 - DNA, 20 bp, Artificial Sequence
  Guide Sequence
cgctccggggacagcgtcag

SEQ ID NO: 185 - DNA, 20 bp, Artificial Sequence
  Guide Sequence
tattcaaacgagagctttga

SEQ ID NO: 186 - DNA, 19 bp, Artificial Sequence
  Guide Sequence
accggagctcttctgcaat

SEQ ID NO: 187 - DNA, 20 bp, Artificial Sequence
  Guide Sequence
cacggcctcatccgtaagta

SEQ ID NO: 188 - DNA, 20 bp, Artificial Sequence
  Guide Sequence
cctcaccttcattgcgccgc

SEQ ID NO: 189 - DNA, 20 bp, Artificial Sequence
  Guide Sequence
gaggaagcagagcggctatg

SEQ ID NO: 190 - DNA, 20 bp, Artificial Sequence
  Guide Sequence
tgtcataggttaacctgctt

SEQ ID NO: 191 - DNA, 20 bp, Artificial Sequence
  Guide Sequence
aagtagtgctgcgaccagac

SEQ ID NO: 192 - DNA, 20 bp, Artificial Sequence
  Guide Sequence
cccgccccgccgcgcattcc

SEQ ID NO: 193 - DNA, 20 bp, Artificial Sequence
  Guide Sequence
aatgaagcgcgggtaaacgg

SEQ ID NO: 194 - DNA, 20 bp, Artificial Sequence
  Guide Sequence
caacctcttgtgtacagagc

SEQ ID NO: 195 - DNA, 20 bp, Artificial Sequence
  Guide Sequence
tgccaggagggctctggaat

SEQ ID NO: 196 - DNA, 20 bp, Artificial Sequence
  Guide Sequence
gaagtggcgcagcgcgcggc

SEQ ID NO: 197 - DNA, 20 bp, Artificial Sequence
  Guide Sequence
gctcccctctgtgaataacc

SEQ ID NO: 198 - DNA, 20 bp, Artificial Sequence
  Guide Sequence
ttcgtcgctacagggttcca

SEQ ID NO: 199 - DNA, 20 bp, Artificial Sequence
  Guide Sequence
gagaagtgcatggacaagcc

SEQ ID NO: 200 - DNA, 19 bp, Artificial Sequence
  Guide Sequence
tcccccacaattatcttaa

SEQ ID NO: 201 - DNA, 20 bp, Artificial Sequence
  Guide Sequence
ggccgcctggcacacgaggt

SEQ ID NO: 202 - DNA, 20 bp, Artificial Sequence
  Guide Sequence
cacaccccaactgtccaaaa

SEQ ID NO: 203 - DNA, 20 bp, Artificial Sequence
  Guide Sequence
tgtgatgcccttagatgtcc

SEQ ID NO: 204 - DNA, 20 bp, Artificial Sequence
  Guide Sequence
ccgtgcggggcgggcaggta

SEQ ID NO: 205 - DNA, 20 bp, Artificial Sequence
  Guide Sequence
cgcggccacgtccagcccca

SEQ ID NO: 206 - DNA, 19 bp, Artificial Sequence
  Guide Sequence
tttaacgaggatccattgg

SEQ ID NO: 207 - DNA, 20 bp, Artificial Sequence
  Guide Sequence
gcggcccccggcccggatga

SEQ ID NO: 208 - DNA, 20 bp, Artificial Sequence
  Guide Sequence
aagttcagcaaatccgctac

SEQ ID NO: 209 - DNA, 20 bp, Artificial Sequence
  Guide Sequence
tgtgcggtccgactgctgtg

SEQ ID NO: 210 - DNA, 20 bp, Artificial Sequence
  Guide Sequence
tcgccggcggtgcggagccg

SEQ ID NO: 211 - DNA, 20 bp, Artificial Sequence
  Guide Sequence
tcgtccacctttgctttctt

SEQ ID NO: 212 - DNA, 20 bp, Artificial Sequence
  Guide Sequence
tcgcccgctgctttaagaac

SEQ ID NO: 213 - DNA, 20 bp, Artificial Sequence
  Guide Sequence
acaaaatgctgtcctgcgcc

SEQ ID NO: 214 - DNA, 21 bp, Artificial Sequence
  Guide Sequence
tgttgctgttactattttctt

SEQ ID NO: 215 - DNA, 20 bp, Artificial Sequence
  Guide Sequence
gatggagtcgtactactcag

SEQ ID NO: 216 - DNA, 20 bp, Artificial Sequence
  Guide Sequence
gaccgcctggctgcgttcta

SEQ ID NO: 217 - DNA, 20 bp, Artificial Sequence
  Guide Sequence
tccctgccctttgtacacac

SEQ ID NO: 218 - DNA, 20 bp, Artificial Sequence
  Guide Sequence
cggaaagacgaaggtcccga

SEQ ID NO: 219 - DNA, 19 bp, Artificial Sequence
  Guide Sequence
cctgtgctaatcctgcaaa

SEQ ID NO: 220 - DNA, 20 bp, Artificial Sequence
  Guide Sequence
aaacaaccagcctacttatt

SEQ ID NO: 221 - DNA, 20 bp, Artificial Sequence
  Guide Sequence
atgaacagcgccagcagcca

SEQ ID NO: 222 - DNA, 20 bp, Artificial Sequence
  Guide Sequence
tcccagccagtgaacacctc

SEQ ID NO: 223 - DNA, 20 bp, Artificial Sequence
  Guide Sequence
cgtcgcagagcatcgcccag

SEQ ID NO: 224 - DNA, 20 bp, Artificial Sequence
  Guide Sequence
cgcggcctcgggcccgaacc

SEQ ID NO: 225 - DNA, 20 bp, Artificial Sequence
  Guide Sequence
gaagtcgcgcccagtaatgc

SEQ ID NO: 226 - DNA, 20 bp, Artificial Sequence
  Guide Sequence
gaaggccccgggcgcaccac

SEQ ID NO: 227 - DNA, 20 bp, Artificial Sequence
  Guide Sequence
cacacctgccttgcctcttg

SEQ ID NO: 228 - DNA, 20 bp, Artificial Sequence
  Guide sequence
ttcctagcaccagtttttag

SEQ ID NO: 229 - DNA, 20 bp, Artificial Sequence
  Guide Sequence
agcataccaatcagctacgc

SEQ ID NO: 230 - DNA, 20 bp, Artificial Sequence
  Guide Sequence
tttggcagcccgtgctattg

SEQ ID NO: 231 - DNA, 20 bp, Artificial Sequence
  Guide Sequence
gctccattggagggcaagtc

SEQ ID NO: 232 - DNA, 20 bp, Artificial Sequence
  Guide sequence
tggagtgggcttcaagaagc

SEQ ID NO: 233 - DNA, 20 bp, Artificial Sequence
  Guide Sequence
ggggtccttgggggtctcag

SEQ ID NO: 234 - DNA, 20 bp, Artificial Sequence
  Guide Sequence
cactgatttcccctcttcac

SEQ ID NO: 235 - DNA, 20 bp, Artificial Sequence
  Guide Sequence
ttcatcctcactgccccccc

SEQ ID NO: 236 - DNA, 20 bp, Artificial Sequence
  Guide Sequence
actttacttgtggtgtgacc

SEQ ID NO: 237 - DNA, 19 bp, Artificial Sequence
  Guide Sequence
ttgtacttcattgctccga

SEQ ID NO: 238 - DNA, 20 bp, Artificial Sequence
  Guide Sequence
tattaaattaaagctcgtcc

SEQ ID NO: 239 - DNA, 20 bp, Artificial Sequence
  Guide Sequence
aagtgctgtgccggctatgc

SEQ ID NO: 240 - DNA, 20 bp, Artificial Sequence
  Guide Sequence
catgattaagagggacggcc

SEQ ID NO: 241 - DNA, 20 bp, Artificial Sequence
  Guide Sequence
gaggggcaactgaggggcag

SEQ ID NO: 242 - DNA, 20 bp, Artificial Sequence
  Guide Sequence
agttacggatccggcttgcc

SEQ ID NO: 243 - DNA, 20 bp, Artificial Sequence
  Guide Sequence
tccatccacgtgggccaagc

SEQ ID NO: 244 - DNA, 20 bp, Artificial Sequence
  Guide Sequence
tgttgatcagcaaaaatgaa

SEQ ID NO: 245 - DNA, 20 bp, Artificial Sequence
  Guide Sequence
ctcaacaactctgacctgat

SEQ ID NO: 246 - DNA, 20 bp, Artificial Sequence
  Guide Sequence
atcacccctccccgcactgt

SEQ ID NO: 247 - DNA, 20 bp, Artificial Sequence
  Guide Sequence
ggggaatgcgagcgctcagt

SEQ ID NO: 248 - DNA, 20 bp, Artificial Sequence
  Guide Sequence
cggcacaatacgaatgcccc

SEQ ID NO: 249 - DNA, 20 bp, Artificial Sequence
  Guide sequence
tatgggcatcgggaagagaa

SEQ ID NO: 250 - DNA, 20 bp, Artificial Sequence
  Guide sequence
cacctcgtcctgctacggga

SEQ ID NO: 251 - DNA, 20 bp, Artificial Sequence
  Guide Sequence
cagggggacttctacttcac

SEQ ID NO: 252 - DNA, 20 bp, Artificial Sequence
  Guide Sequence
tgcgggcactacggctgaga

SEQ ID NO: 253 - DNA, 20 bp, Artificial Sequence
  Guide Sequence
ggggagggcgggagcgatag

SEQ ID NO: 254 - DNA, 20 bp, Artificial Sequence
  Guide Sequence
cacggcctcatccgtaagta

SEQ ID NO: 255 - DNA, 20 bp, Artificial Sequence
  Guide Sequence
acccgagattgagcaataac

SEQ ID NO: 256 - DNA, 20 bp, Artificial Sequence
  Guide Sequence
cctcttcggtacctcctcag

SEQ ID NO: 257 - DNA, 20 bp, Artificial Sequence
  Guide Sequence
tcccctcgggtccattatcg

SEQ ID NO: 258 - DNA, 20 bp, Artificial Sequence
  Guide Sequence
agctgtacttgtggctgagc

SEQ ID NO: 259 - DNA, 20 bp, Artificial Sequence
  Guide Sequence
ttcggggttctccgccatgg

SEQ ID NO: 260 - DNA, 20 bp, Artificial Sequence
  Guide Sequence
gcctgccgggactgggctgc

SEQ ID NO: 261 - DNA, 20 bp, Artificial Sequence
  Guide Sequence
tgcaaaaaaccaggctggac

SEQ ID NO: 262 - DNA, 19 bp, Artificial Sequence
  Guide Sequence
catgattaagagggacggc

SEQ ID NO: 263 - DNA, 21 bp, Artificial Sequence
  Guide Sequence
gggagcggcggccgtggcggc

SEQ ID NO: 264 - DNA, 19 bp, Artificial Sequence
  Guide Sequence
tcggtgaagtccccaaaat

SEQ ID NO: 265 - DNA, 20 bp, Artificial Sequence
  Guide Sequence
tcgacgatggcacgtctgat

SEQ ID NO: 266 - DNA, 20 bp, Artificial Sequence
  Guide Sequence
ccgtcccgcgaggacttcga

SEQ ID NO: 267 - DNA, 20 bp, Artificial Sequence
  Guide Sequence
aacatctctcccttctcctt

SEQ ID NO: 268 - DNA, 20 bp, Artificial Sequence
  Guide sequence
gaggaagacaccgtccccac

SEQ ID NO: 269 - DNA, 20 bp, Artificial Sequence
  Guide Sequence
cccgcccgcgctccgcgcac

SEQ ID NO: 270 - DNA, 20 bp, Artificial Sequence
  Guide sequence
cgcctgtgtgattactctat

SEQ ID NO: 271 - DNA, 20 bp, Artificial Sequence
  Guide Sequence
ggcgctcttccgggggtatt

SEQ ID NO: 272 - DNA, 20 bp, Artificial Sequence
  Guide Sequence
gactaacattcctcaaaccc

SEQ ID NO: 273 - DNA, 20 bp, Artificial Sequence
  Guide Sequence
cgttccgaagggacgggcga

SEQ ID NO: 274 - DNA, 20 bp, Artificial Sequence
  Guide Sequence
ggcggaagcagcgaacagag

SEQ ID NO: 275 - DNA, 20 bp, Artificial Sequence
  Guide Sequence
ccaaagccaatcggtcacat

SEQ ID NO: 276 - DNA, 20 bp, Artificial Sequence
  Guide Sequence
ccgttaagaggtaaacgggt

SEQ ID NO: 277 - DNA, 20 bp, Artificial Sequence
  Guide Sequence
atgcatgtctaagtacacac

SEQ ID NO: 278 - DNA, 20 bp, Artificial Sequence
  Guide Sequence
tccggcaagtccaccaccac

SEQ ID NO: 279 - DNA, 20 bp, Artificial Sequence
  Guide Sequence
tccgcaccgccggcgacggc

SEQ ID NO: 280 - DNA, 20 bp, Artificial Sequence
  Guide Sequence
cgttccctccgcttcgaccc

SEQ ID NO: 281 - DNA, 20 bp, Artificial Sequence
  Guide Sequence
tggacccctacagtatgttc

SEQ ID NO: 282 - DNA, 20 bp, Artificial Sequence
  Guide Sequence
cgaatacagaccgtgaaagc

SEQ ID NO: 283 - DNA, 20 bp, Artificial Sequence
  Guide Sequence
catcgggaagagaaagggta

SEQ ID NO: 284 - DNA, 33 bp, Artificial Sequence
  3′ linker I
ctgctgacttcagtggttctagaggtgtccaac